SEQUENCE LISTING (117 sequences)

SEQ ID NO: 1 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 2 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 3 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 4 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 5 - protein, 107 aa, Mus musculus
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 6 - protein, 119 aa, Mus musculus
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 7 - protein, 107 aa, Artificial Sequence [sequence omitted]

SEQ ID NO: 8 - protein, 119 aa, Artificial Sequence [sequence omitted]

SEQ ID NO: 9 - protein, 107 aa, Artificial Sequence
  Description of Artificial Sequence Synthetic polypeptide
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 10 - protein, 119 aa, Artificial Sequence
  Description of Artificial Sequence Synthetic polypeptide
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 11 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 217 aa, Artificial Sequence
  Description of Artificial Sequence Synthetic polypeptide
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 122 aa, Mus musculus
QIHLVQSGPELKKPGETVKISCKASGYTFTNYGMNWVKQAPGKGLKWMGWINTYTGEPTYADDFKGRFAFSLETSASTAYLQINNLKNEDMATYFCARSWYYVSNYWYFDVWGQGTLVTVSS

SEQ ID NO: 18 - protein, 117 aa, Homo sapiens
EVQLVQSGAEVKKPGASVKVSCKASGYTFTSYYIHWVRQAPGQGLEWIGWINPGSGNTNYAQKFQGRVTITRDTSTSTAYLELSSLRSEDTAVYYCARDTAAYFDYWGQGTLVTVSS

SEQ ID NO: 19 - protein, 117 aa, Homo sapiens
EVQLVESGGGLVQPGGSLRLSCAASGFTFSSYAMSWVRQAPGKGLEWVGAISSSGSSTYYADSVKGRFTISRDNSKNTLYLQMNSLRAEDTAVYYCARDTAAYFDYWGQGTLVTVSS

SEQ ID NO: 20 - protein, 117 aa, Homo sapiens
EVQLVQSGSELKKPGASVKVSCKASGYTFTSYYIHWVRQAPGQGLEWIGWINPGSGNTNYAQKFQGRFVFSLDTSVSTAYLQISSLKAEDTAVYYCARDTAAYFDYWGQGTLVTVSS

SEQ ID NO: 21 - protein, 116 aa, Mus musculus
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 22 - protein, 109 aa, Homo sapiens
SYELTQPPSVSVSPGQTARITCQGDALRSYYASWYQQKPGQAPVLVIYKDNNRPSGIPERFSGSSSGNTATLTISGAQAEDEADYYCNSRDSSGNHVVFGGGTKLTVLG

SEQ ID NO: 23 - protein, 112 aa, Homo sapiens
QPVLTQSPSASASLGASVKLTCTLSSGHSSYTIAWHQQQPGKGPRYLMKLNSDGSHSKGDGIPDRFSGSSSGADRYLTISNLQSEDEADYYCGTWGTGIVVFGGGTKLTVLG

SEQ ID NO: 24 - protein, 12 aa, Mus musculus
LKKDGSHSTGDG

SEQ ID NO: 25 - protein, 12 aa, Artificial Sequence
  Description of Artificial Sequence Synthetic mutated mouse sequence
LKKDASHSTGDG

SEQ ID NO: 26 - protein, 12 aa, Artificial Sequence
  Description of Artificial Sequence Synthetic mutated mouse sequence
LKKEGSHSTGDG

SEQ ID NO: 27 - protein, 12 aa, Artificial Sequence
  Description of Artificial Sequence Synthetic mutated mouse sequence
LKKTGSHSTGDG

SEQ ID NO: 28 - protein, 12 aa, Artificial Sequence
  Description of Artificial Sequence Synthetic mutated mouse sequence
LKKDGSHSTGDA

SEQ ID NO: 29 - protein, 12 aa, Artificial Sequence
  Description of Artificial Sequence Synthetic mutated mouse sequence
LKKDGSHSTGEG

SEQ ID NO: 30 - protein, 12 aa, Artificial Sequence
  Description of Artificial Sequence Synthetic mutated mouse sequence
LKKDGSHSTGTG

SEQ ID NO: 31 - protein, 12 aa, Artificial Sequence
  Description of Artificial Sequence Synthetic mutated mouse sequence
LKKSGSHSTGDG

SEQ ID NO: 32 - protein, 12 aa, Artificial Sequence
  Description of Artificial Sequence Synthetic mutated mouse sequence
LKKSGSHSTGSG

SEQ ID NO: 33 - protein, 12 aa, Artificial Sequence
  Description of Artificial Sequence Synthetic mutated mouse sequence
LKKTGSHSTGDA

SEQ ID NO: 34 - protein, 116 aa, Artificial Sequence
  Description of Artificial Sequence Synthetic Humanized 8G8 Immunoglobulin Light Chain polypeptide
QPVLTQSPSASASLGASVKLTCTLSSQHSTYTIEWHQQQPGKGPRYLMKLKKDGSHSTGDGIPDRFSGSSSGADRYLTISNLQSEDEADYYCGVGDTIKEQFVYVFGGGTKLTVLG

SEQ ID NO: 35 - protein, 122 aa, Artificial Sequence
  Description of Artificial Sequence Synthetic Humanized 8G8 Immunoglobulin heavy chain polypeptide
EVQLVQSGAEVKKPGASVKVSCKASGYTFTNYGMNWVRQAPGQGLEWIGWINTYTGEPTYADDFKGRVTITRDTSTSTAYLELSSLRSEDTAVYYCARSWYYVSNYWYFDVWGQGTLVTVSS

SEQ ID NO: 36 - DNA, 366 bp, Artificial Sequence
  Description of Artificial Sequence Synthetic Humanized 8G8 Immunoglobulin heavy chain polynucleotide
gaggttcagctggtgcagtctggcgccgaagtgaaaaaaccaggggcctcagtgaaagtgtcctgtaaagcttctggctacaccttcaccaactacggcatgaactgggtgcgtcaggccccgggtcagggcctggaatggatcggctggatcaacacctacaccggcgaaccaacctacgccgatgatttcaaaggccgtgtgactataacccgtgacacctccaccagcacagcctacctagaactgagcagcttaagaagcgaggacactgccgtctattattgcgcgcgtagctggtactacgtgagcaactactggtacttcgatgtgtggggtcaaggaaccctggtcaccgtctcctcg

SEQ ID NO: 37 - protein, 122 aa, Artificial Sequence
  Description of Artificial Sequence Synthetic Humanized 8G8 Immunoglobulin Heavy Chain polypeptide
EVQLVESGGGLVQPGGSLRLSCAASGYTFTNYGMNWVRQAPGKGLEWVGWINTYTGEPTYADDFKGRFTISRDNSKNTLYLQMNSLRAEDTAVYYCARSWYYVSNYWYFDVWGQGTLVTVSS

SEQ ID NO: 38 - protein, 115 aa, Artificial Sequence
  Description of Artificial Sequence Synthetic Humanized 8G8 Immunoglobulin Light Chain (mutated) polypeptide
SVLTQSPSASASLGASVKLTCTLSSQHSTYTIEWYQQQPGKGPRYLMKLKKDGSHSTGDGIPDRFSGSSSGADRYLTISNLQSEDEADYYCGVGDTIKEQFVYVFGGGTKLTVLG

SEQ ID NO: 39 - DNA, 345 bp, Artificial Sequence
  Description of Artificial Sequence Synthetic Humanized 8G8 Immunoglobulin Light Chain (mutated) polynucleotide
tctgtgctgacccagagcccaagcgccagcgccagcctgggcgccagcgtgaaactgacctgcaccctgagcagccagcacagcacctacaccatcgaatggtatcagcagcagccaggcaaaggcccacgctacctgatgaaactgaaaaaagatggcagccacagcaccggcgatggcatcccagatcgcttcagcggcagcagcagcggcgccgatcgctacctgaccatcagcaacctgcagagcgaagatgaagccgattactactgcggcgtgggcgataccatcaaagaacagttcgtgtacgtgttcggcggcggtaccaaactgaccgtgctgggc

SEQ ID NO: 40 - protein, 112 aa, Homo sapiens
DIVMTQSPLSLSVTPGEPASISCRSSQSLLHTNGYNYLDWYVQKPGQSPQLLIYLASNRASGVPDRFSGSGSGTDFTLKISRVETEDVGVYYCMQALQIPRTFGQGTKVEIK

SEQ ID NO: 41 - protein, 130 aa, Homo sapiens
EEQVLESGGGLVKPGGSLRLSCAASGFTFSPYSVFWVRQAPGKGLEWVSSINSDSTYKYYADSVKGRFTISRDNAENSIFLQMNSLRAEDTAVYYCARDRSYYAFSSGSLSDYYYGLDVWGQGTLVTVSS

SEQ ID NO: 42 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSNSRYKYYADSVKGRFTISRDNAENSIFLQMNSLRAEDTAVYYCARDRSYYAFSSGSLSDYYYGLDVWGQGTLVTVSS

SEQ ID NO: 43 - protein, 18 aa, Homo sapiens
SSINSDSTYKYYADSVKG

SEQ ID NO: 44 - protein, 18 aa, Artificial Sequence
  Description of Artificial Sequence Synthetic Affinity-matured human Ig Heavy Chain peptide
SSIXSXSXYKYYADSVKG

SEQ ID NO: 45 - protein, 743 aa, Human cytomegalovirus
MRPGLPPYLTVFTVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKAQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 46 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 47 - protein, 171 aa, Human cytomegalovirus
MSPKNLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 48 - protein, 214 aa, Human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVEKSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 49 - protein, 76 aa, Human cytomegalovirus
MCMMSHNKAFFLSLQHAAVSGVAVCLSVRRGAGSVPAGNRGKKTIITEYRITGTRALARCPTKPVTSMWNSSWTSR

SEQ ID NO: 50 - protein, 472 aa, Human cytomegalovirus
MGKKEMIMVKGIPKIMLLISITFLLLSLINCNVLVNSRGTRRSWPYTVLSYRGKEILKKQKEDILKRLMSTSSDGYRFLMYPSQQKFHAIVISMDKFPQDYILAGPIRNDSITHMWFDFYSTQLRKPAKYVYSEYNHTAHKITLRPPPCGTVPSMNCLSEMLNVSKRNDTGEKGCGNFTTFNPMFFNVPRWNTKLYIGSNKVNVDSQTIYFLGLTALLLRYAQRNCTRSFYLVNAMSRNLFRVPKYINGTKLKNTMRKLKRKQALVKEQPQKKNKKSQSTTTPYLSYTTSTAFNVTTNVTYSATAAVTRVATSTTGYRPDSNFMKSIMATQLRDLATWVYTTLRYRNEPFCKPDRNRTAVSEFMKNTHVLIRNETPYTIYGTLDMSSLYYNETMSVENETASDNNETTPTSPSTRFQRTFIDPLWDYLDSLLFLDKIRNFSLQLPAYGNLTPPEHRRAANLSTLNSLWWWSQ

SEQ ID NO: 51 - protein, 743 aa, Human cytomegalovirus
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 52 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 53 - protein, 464 aa, Human cytomegalovirus
MGRKEDMRSISKLFFIISLTVLLFSIINCKVVRPPGRYWLGTVLSTIGKQKLDKFKLEILKQLEREPYTKYFNMTRQHVKNLTMNMTQFPQYYILAGPIRNDSITYLWFDFYSTQLRKPAKYVYSQYNHTAKTITFRPPSCGTVPSMTCLSEMLNVSKRNDTGEQGCGNFTTFNPMFFNVPRWNTKLYVGPTKVNVDSQTIYFLGLTALLLRYAQRNCTHSFYLVNAMSRNLFRVPKYINGTKLKNTMRKLKRKQAPVKEQLEKKTKKSQSTTTPYFSYTTSTALNVTTNATYRVTTSAKRIPTSTIAYRPDSSFMKSIMATQLRDLATWVYTTLRYRNEPFCKPDRNRTAVSEFMKNTHVLIRNETPYTIYGTLDMSSLYYNETMSVENETASDNNETTPTSPSTRFQKTFIDPLWDYLDSLLFLDKIRNFSLQLPAYGNLTPPEHRRAVNLSTLNSLWWWLQ

SEQ ID NO: 54 - protein, 10 aa, Mus musculus
GYTFTNYGMN

SEQ ID NO: 55 - protein, 17 aa, Mus musculus
WINTYTGEPTYADDFKG

SEQ ID NO: 56 - protein, 13 aa, Mus musculus
SWYYVSNYWYFDV

SEQ ID NO: 57 - protein, 12 aa, Mus musculus
TLSSQHSTYTIE

SEQ ID NO: 58 - protein, 11 aa, Mus musculus
LKKDGSHSTGD

SEQ ID NO: 59 - protein, 11 aa, Artificial Sequence
  Description of Artificial Sequence Synthetic mutated mouse HVR-L2 peptide
LKKDASHSTGD

SEQ ID NO: 60 - protein, 11 aa, Artificial Sequence
  Description of Artificial Sequence Synthetic mutated mouse HVR-L2 peptide
LKKEGSHSTGD

SEQ ID NO: 61 - protein, 11 aa, Artificial Sequence
  Description of Artificial Sequence Synthetic mutated mouse HVR-L2 peptide
LKKTGSHSTGD

SEQ ID NO: 62 - protein, 11 aa, Artificial Sequence
  Description of Artificial Sequence Synthetic mutated mouse HVR-L2 peptide
LKKDGSHSTGD

SEQ ID NO: 63 - protein, 11 aa, Artificial Sequence
  Description of Artificial Sequence Synthetic mutated mouse HVR-L2 peptide
LKKDGSHSTGE

SEQ ID NO: 64 - protein, 11 aa, Artificial Sequence
  Description of Artificial Sequence Synthetic mutated mouse HVR-L2 peptide
LKKDGSHSTGT

SEQ ID NO: 65 - protein, 11 aa, Artificial Sequence
  Description of Artificial Sequence Synthetic mutated mouse HVR-L2 peptide
LKKSGSHSTGD

SEQ ID NO: 66 - protein, 11 aa, Artificial Sequence
  Description of Artificial Sequence Synthetic mutated mouse HVR-L2 peptide
LKKSGSHSTGS

SEQ ID NO: 67 - protein, 11 aa, Artificial Sequence
  Description of Artificial Sequence Synthetic mutated mouse HVR-L2 peptide
LKKTGSHSTGD

SEQ ID NO: 68 - protein, 13 aa, Mus musculus
GVGDTIKEQFVYV

SEQ ID NO: 69 - protein, 12 aa, Artificial Sequence
  Description of Artificial Sequence Synthetic Consensus HVR-L2 Sequence
LKKXXSHSTGXX

SEQ ID NO: 70 - protein, 25 aa, Homo sapiens
EVQLVQSGAEVKKPGASVKVSCKAS

SEQ ID NO: 71 - protein, 14 aa, Homo sapiens
WVRQAPGQGLEWIG

SEQ ID NO: 72 - protein, 32 aa, Homo sapiens
RVTITRDTSTSTAYLELSSLRSEDTAVYYCAR

SEQ ID NO: 73 - protein, 11 aa, Homo sapiens
WGQGTLVTVSS

SEQ ID NO: 74 - protein, 14 aa, Mus musculus
WVKQAPGKGLKWMG

SEQ ID NO: 75 - protein, 32 aa, Mus musculus
RFAFSLETSASTAYLQINNLKNEDMATYFCAR

SEQ ID NO: 76 - protein, 11 aa, Mus musculus
WGQGTLVTVSS

SEQ ID NO: 77 - protein, 25 aa, Homo sapiens [sequence omitted]

SEQ ID NO: 78 - protein, 14 aa, Homo sapiens
WVRQAPGKGLEWVG

SEQ ID NO: 79 - protein, 32 aa, Homo sapiens
RFTISRDNSKNTLYLQMNSLRAEDTAVYYCAR

SEQ ID NO: 80 - protein, 25 aa, Homo sapiens
EVQLVQSGSELKKPGASVKVSCKAS

SEQ ID NO: 81 - protein, 32 aa, Homo sapiens
RFVFSLDTSVSTAYLQISSLKAEDTAVYYCAR

SEQ ID NO: 82 - protein, 22 aa, Mus musculus
QLVLTQSSSASFSLGASAKLTC

SEQ ID NO: 83 - protein, 15 aa, Mus musculus
WYQQQPLKPPKYVME

SEQ ID NO: 84 - protein, 32 aa, Mus musculus
GIPDRFSGSSSGADRYLSISNIQPEDEAIYIC

SEQ ID NO: 85 - protein, 11 aa, Mus musculus
FGGGTKVTVLG

SEQ ID NO: 86 - protein, 22 aa, Homo sapiens
QPVLTQSPSASASLGASVKLTC

SEQ ID NO: 87 - protein, 15 aa, Homo sapiens
WHQQQPGKGPRYLMK

SEQ ID NO: 88 - protein, 32 aa, Homo sapiens
GIPDRFSGSSSGADRYLTISNLQSEDEADYYC

SEQ ID NO: 89 - protein, 11 aa, Homo sapiens
FGGGTKLTVLG

SEQ ID NO: 90 - protein, 21 aa, Homo sapiens
SVLTQSPSASASLGASVKLTC

SEQ ID NO: 91 - protein, 15 aa, Homo sapiens
WYQQQPGKGPRYLMK

SEQ ID NO: 92 - protein, 32 aa, Artificial Sequence
  Description of Artificial Sequence Synthetic Mutant mouse FR3 polypeptide
AIPDRFSGSSSGADRYLSISNIQPEDEAIYIC

SEQ ID NO: 93 - protein, 32 aa, Artificial Sequence
  Description of Artificial Sequence Synthetic mutated Human lambda 4 FR3 polypeptide
AIPDRFSGSSSGADRYLTISNLQSEDEADYYC

SEQ ID NO: 94 - protein, 214 aa, Human cytomegalovirus
MLRLLLRHHFHCLLLCAVWATPCLASPWFTLTANQNPSPPWSKLTYPKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 95 - protein, 122 aa, Artificial Sequence
  Description of Artificial Sequence Synthetic Humanized 8G8 Immunoglobulin Heavy Chain polypeptide
EVQLVQSGSELKKPGASVKVSCKASGYTFTNYGMNWVRQAPGQGLEWIGWINTYTGEPTYADDFKGRFVFSLDTSVSTAYLQISSLKAEDTAVYYCARSWYYVSNYWYFDVWGQGTLVTVSS

SEQ ID NO: 96 - protein, 171 aa, Human cytomegalovirus; identical to SEQ ID NO: 47

SEQ ID NO: 97 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 98 - protein, 28 aa, Artificial Sequence
  Description of Artificial Sequence Synthetic peptide
SRALPDQTRYKYVEQLVDLTLNYHYDAS

SEQ ID NO: 99 - protein, 10 aa, Homo sapiens
GFTFSPYSVF

SEQ ID NO: 100 - protein, 18 aa, Homo sapiens
SSINSNSTYKYYADSVKG

SEQ ID NO: 101 - protein, 18 aa, Homo sapiens
SSINSDSRYKYYADSVKG

SEQ ID NO: 102 - protein, 18 aa, Homo sapiens
SSINSNSRYKYYADSVKG

SEQ ID NO: 103 - protein, 23 aa, Homo sapiens
ARDRSYYAFSSGSLSDYYYGLDV

SEQ ID NO: 104 - protein, 16 aa, Homo sapiens
RSSQSLLHTNGYNYLD

SEQ ID NO: 105 - protein, 7 aa, Homo sapiens
LASNRAS

SEQ ID NO: 106 - protein, 9 aa, Homo sapiens
MQALQIPRT

SEQ ID NO: 107 - protein, 25 aa, Homo sapiens
EEQVLESGGGLVKPGGSLRLSCAAS

SEQ ID NO: 108 - protein, 13 aa, Homo sapiens
WVRQAPGKGLEWV

SEQ ID NO: 109 - protein, 30 aa, Homo sapiens
RFTISRDNAENSIFLQMNSLRAEDTAVYYC

SEQ ID NO: 110 - protein, 11 aa, Homo sapiens
WGQGTLVTVSS

SEQ ID NO: 111 - protein, 23 aa, Homo sapiens
DIVMTQSPLSLSVTPGEPASISC

SEQ ID NO: 112 - protein, 15 aa, Homo sapiens
WYVQKPGQSPQLLIY

SEQ ID NO: 113 - protein, 32 aa, Homo sapiens
GVPDRFSGSGSGTDFTLKISRVETEDVGVYYC

SEQ ID NO: 114 - protein, 10 aa, Homo sapiens
FGQGTKVEIK

SEQ ID NO: 115 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSNSTYKYYADSVKGRFTISRDNAENSIFLQMNSLRAEDTAVYYCARDRSYYAFSSGSLSDYYYGLDVWGQGTLVTVSS

SEQ ID NO: 116 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSDSRYKYYADSVKGRFTISRDNAENSIFLQMNSLRAEDTAVYYCARDRSYYAFSSGSLSDYYYGLDVWGQGTLVTVSS

SEQ ID NO: 117 - DNA, 39 bp, Homo sapiens
  CDS (1)..(39)
gtggtgttcggcggcggtaccaagctgaccgtccttggc

(19) United States
(12) Patent Application Publication
(10) Pub. No.: US 2013/0195851 A1
(43) Pub. Date: Aug. 1, 2013

(54) ARTICLES OF MANUFACTURE AND METHODS FOR CO-ADMINISTRATION OF ANTIBODIES

(71) Applicant: Genentech, Inc., South San Francisco, CA (US)

(72) Inventors: Sreedhara Alavattam, Fremont, CA (US); Christina H. de Toledo Pelizon, Oakville (CA); Zephania Kwong Glover, Martinez, CA (US); Lada Mitchell, Allschwil (CH)

(73) Assignee: GENENTECH, INC., South San Francisco, CA (US)

(21) Appl. No.: 13/723,889

(22) Filed: Dec. 21, 2012

Related U.S. Application Data
(60) Provisional application No. 61/579,904, filed on Dec. 23, 2011.

(51) Int. Cl. A61K 39/42; A61K 39/395

(52) U.S. Cl. CPC A61K 39/42; A61K 39/39558; A61K 39/3955
USPC 424/133.1; 424/141.1; 424/155.1; 424/147.1; 424/145.1; 424/156.1; 424/148.1; 424/149.1

(57) ABSTRACT

The present invention relates to articles of manufacture and methods for co-administration of antibodies and/or antibody-like molecules, and further concerns methods for intravenous administration of more than one antibody and/or antibody-like molecule to a subject in need from a stable mixture contained in the same article of manufacture, such as an intravenous infusion bag (IV bag).

This non-provisional application filed under 37 CFR §1.53(b), claims the benefit under 35 USC §119(e) of U.S. Provisional Application Ser. No. 61/579,904, filed on Dec. 23, 2011, which is incorporated by reference in entirety.

### FIELD OF THE INVENTION

The present invention concerns articles of manufacture and method for co-administration of antibodies and/or antibody-like molecules. In particular, the invention concerns an article of manufacture for intravenous use, such as an intravenous infusion bag (IV bag), comprising a stable mixture of more than one antibody and/or antibody-like molecule. The invention further concerns methods for intravenous administration of more than one antibody and/or antibody-like molecule to a subject in need from a stable mixture contained in the same article of manufacture, such as an intravenous infusion bag (IV bag).

### BACKGROUND OF THE INVENTION

Monoclonal antibodies have emerged as effective therapeutic agents for the treatment of various diseases, including a variety of malignancies.

Thus, a recombinant humanized version of the murine anti-HER2 antibody 4D5 (huMAb4D5-8, rhuMAb HER2, Trastuzumab or HERCEPTIN®; U.S. Pat. No. 5,821,337) received marketing approval from the Food and Drug Administration Sep. 25, 1998 for the treatment of patients with metastatic breast cancer whose tumors overexpress the HER2 protein. At present, Trastuzumab is approved for use as a single agent or in combination with chemotherapy or hormone therapy in the metastatic setting, and as single agent or in combination with chemotherapy as adjuvant treatment for patients with early-stage HER2-positive breast cancer. Trastuzumab-based therapy is now the recommended treatment for patients with HER2-positive early-stage breast cancer who do not have contraindications for its use (Herceptin® prescribing information; NCCN Guidelines, version 2.2011). Trastuzumab plus Docetaxel (or paclitaxel) is a registered standard of care in the first-line metastatic breast cancer (MBC) treatment setting (Slamon et al. N Engl J Med. 2001; 344(11):783-792.; Marty et al. J Clin Oncol. 2005; 23(19):4265-4274).

Another anti-HER2 monoclonal antibody, Pertuzumab (also known as recombinant humanized monoclonal antibody 2C4 (rhuMAb 2C4); Genentech, Inc, South San Francisco) represents the first in a new class of agents known as HER dimerization inhibitors (HDI) and functions to inhibit the ability of HER2 to form active heterodimers or homodimers with other HER receptors (such as EGFR/HER1, HER2, HER3 and HERO). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003).

Pertuzumab blockade of the formation of HER2-HER3 heterodimers in tumor cells has been demonstrated to inhibit critical cell signaling, which results in reduced tumor proliferation and survival (Agus et al. Cancer Cell 2:127-37 (2002)).

Pertuzumab has undergone testing as a single agent in the clinic with a phase Ia trial in patients with advanced cancers and phase II trials in patients with ovarian cancer and breast cancer as well as lung and prostate cancer. In a Phase I study, patients with incurable, locally advanced, recurrent or metastatic solid tumors that had progressed during or after standard therapy were treated with Pertuzumab given intravenously every 3 weeks. Pertuzumab was generally well tolerated. Tumor regression was achieved in 3 of 20 patients evaluable for response. Two patients had confirmed partial responses. Stable disease lasting for more than 2.5 months was observed in 6 of 21 patients (Agus et al. Pro Am Soc Clin Oncol 22:192 (2003)). At doses of 2.0-15 mg/kg, the pharmacokinetics of Pertuzumab was linear, and mean clearance ranged from 2.69 to 3.74 mL/day/kg and the mean terminal elimination half-life ranged from 15.3 to 27.6 days. Antibodies to Pertuzumab were not detected (Allison et al. Pro Am Soc Clin Oncol 22:197 (2003)).

Other monoclonal antibodies approved for use in oncology include, for example, the anti-epidermal growth factor receptor (anti-EGFR) monoclonal antibody ERBITUX® (cetuximab) (Figlin R A, Proceedings ASCO. 2002; 21:35. Abstract); the anti-CD20 monoclonal antibody RITUXAN® (rituximab) (Kaminsky et al., Blood 2000 96(4):1259-66); and the anti-vascular endothelial growth factor (anti-VEGF) antibody AVASTIN® (bevacizumab) (Hurwitz et al., N. Engl. J. Med. 2004; 350(23):2335-42. Monoclonal antibodies are also in development or clinical use for the treatment of other diseases. For example, rituximab has also been shown to be effective in the treatment of certain autoimmune diseases, such as rheumatoid arthritis (Edwards et al.,N Engl J Med 350 (25): 2572-81).

While monoclonal antibodies have been highly effective as single agents for various disease indications, a number of antibody combinations have also been evaluated.

US 2006/0034842 describes methods for treating ErbB-expressing cancer with anti-ErbB2 antibody combinations. US 2008/0102069 describes the use of Trastuzumab and Pertuzumab in the treatment of HER2-positive metastatic cancer, such as breast cancer. Baselga et al., J Clin Oncol, 2007 ASCO Annual Meeting Proceedings Part I, Col. 25, No. 18S (June 20 Supplement), 2007:1004 report the treatment of patients with pre-treated HER2-positive breast cancer, which has progressed during treatment with Trastuzumab, with a combination of Trastuzumab and Pertuzumab. Portera et al., J Clin Oncol, 2007 ASCO Annual Meeting Proceedings Part I. Vol. 25, No. 18S (June 20 Supplement), 2007:1028 evaluated the efficacy and safety of Trastuzumab+Pertuzumab combination therapy in HER2-positive breast cancer patients, who had progressive disease on Trastuzumab-based therapy. The authors concluded that further evaluation of the efficacy of combination treatment was required to define the overall risk and benefit of this treatment regimen. Pertuzumab has been evaluated in Phase II studies in combination with Trastuzumab in patients with HER2-positive metastatic breast cancer who have previously received Trastuzumab for metastatic disease. One study, conducted by the National cancer Institute (NCI), enrolled 11 patients with previously treated HER2-positive metastatic breast cancer. Two out of the 11 patients exhibited a partial response (PR) (Baselga et al., J Clin Oncol 2007 ASCO Annual Meeting Proceedings; 25:18S (June 20 Supplement): 1004, The results of a Phase II neoadjuvant study evaluating the effect of a novel combination regimen of Pertuzumab and Trastuzumab plus chemotherapy (docetaxel) in women with early-stage HER2-positive breast cancer, presented at the CTRC-AACR San Antonio Breast Cancer Symposium (SABCS), Dec. 8-12, 2010, showed that the two HER2 antibodies plus docetaxel given in the neoadjuvant setting prior to surgery significantly improved the rate of complete tumor disappearance (pathological complete response rate, pCR, of 45.8 percent) in the breast by more than half compared to Trastuzumab plus docetaxel (pCR of 29. 0 percent), p=0.014.

Other antibody combinations in various stages of testing including combinations of monoclonal antibodies to HER family members, such as EGFR and HER2 (Mendelsohn et al., Oncogene 1999, 18:731-8; Kawaguchi et al., Br. J. Cancer 2007, 97:494-501; Half et al., Cancer Lett 2007; 251:237-46; Meira et al., Br. J. Cancer 2009; 101:782-91; Larbourer et al., Clin Cancer Res 2007; 13:3356-62); and EGFR and HER3 (Schoeberl et al., Cancer Res 2010; 70:13:3356-62).

Clinical trials testing the safety or efficacy of anti-EGFR antibodies combined with anti-HER2 or anti-HER3 antibodies are in progress. Similarly, clinical trials investigating the safety or efficacy of anti-EGFR and anti-IGF-1R monoclonal antibodies are ongoing.

Monoclonal antibody combinations targeting tumor angiogenesis are also being tested. Thus, for example, combinations of VEGF-pathway inhibitors and combinations inhibiting both VEGF-pathway and non-VEGF-pathway angiogenesis are being evaluated.

Phase 1 and 2 clinical trials with the anti-CD20 monoclonal antibody rituximab and antibodies targeting the B-cell antigens CD22, CD80 and CD52, respectively demonstrated that these combinations were well tolerated and resulted in clinical responses equal t or greater that single-agent therapy (Leonard et al., Ann Oncol 2007; 18:1216-23; Leonard et al., J Clin Oncol 2005; 23:5044-51; Leonard et al., Cancer 2008; 113:2714-23; Faderl et al., Cancer 2010; 116:2360-5).

For further details of emerging antibody combinations in oncology see, e.g. Demarest et al., mAbs 2011; 3:4, 338-351 (Landes Bioscience), the entire disclosure of which is expressly incorporated by reference herein.

These and other promising combinations of antibody therapeutics create a need for more efficient and convenient modes of administration.

### SUMMARY OF THE INVENTION

In one aspect, the invention concerns an article of manufacture containing a stable liquid mixture of more than one monoclonal antibody, formulated separately, suitable for intravenous administration to a patient in need.

In another aspect, the invention concerns a method for intravenous administration of at least two antibodies and/or antibody-like molecules, wherein said antibodies and/or antibody-like molecules are formulated separately and are administered from a stable liquid mixture contained in a single intravenous (IV) bag.

In both aspects, the invention encompasses, but is not limited to, the following specific embodiments:

In one embodiment, the article of manufacture is an intravenous (IV) bag.

In another embodiment, at least one antibody is a naked antibody.

In yet another embodiment, all antibodies are naked antibodies.

In a further embodiment, at least one antibody is an anti-cancer antibody.

In a still further embodiment, all antibodies are anti-cancer antibodies.

In a different embodiment, at least one antibody is an anti-viral antibody.

In a more specific embodiment, all antibodies are anti-viral antibodies.

In another embodiment, at least one antibody is infused for at least about 90 minutes when administered individually.

In yet another embodiment, at least one antibody is infused for at least about 120 minutes when administered individually.

In a further embodiment, at least one antibody is infused for about 90 minutes to about 10 hours when administered individually.

In a still further embodiment, each antibody present in the mixture is infused for at least about 120 minutes when administered individually.

In an additional embodiment, the IV bag contains two antibodies.

In another embodiment, the mixture is stable for at least about 4 to 6 hours at 2 to 8° C. or 15 to 30° C.

In another embodiment, the mixture is stable for at least about 8 hours at 2 to 8° C. or 15 to 30° C.

In another embodiment, the mixture is stable for at least about 12 hours at 2 to 8° C. or 15 to 30° C.

In another embodiment, the mixture is stable for at least about 24 hours at 2 to 5° C. or 15 to 30° C.

In another embodiment, stability is measured at 5° C. or at 30° C.

In another embodiment, the mixture is in a saline solution.

In a further embodiment, the mixture is in a dextrose solution.

In a further embodiment, the saline solution comprises about 0.9% NaCl or about 0.45% NaCl.

In a further embodiment, IV bag is a polyolefin or polyvinyl chloride infusion bag.

In a still further embodiment, the polyolefin is polypropylene or polyethylene.

In a different embodiment, is evaluated by an assay selected from the group consisting of: color, appearance and clarity (CAC), concentration and turbidity analysis, particulate analysis, size exclusion chromatography (SEC), ion-exchange chromatography (IEC), reverse phase HPL, hydrophobic interaction chromatography, HIAC-Royco, capillary zone electrophoresis (CZE), image capillary isoelectric focusing (iCIEF), and potency assay.

In another embodiment, at least one monoclonal antibody binds to an antigen selected from the group consisting of EGFR, HER2, HER3, HER4, CD20, CD22, IL-8, CD40, CD11a, IgE, VEGF, STIgMA, CD 18, Apo-2 receptor, TNF-α, Tissue Factor (TF), human α₄-β₇ integrin, CD3, CD25, CD52, CD33, CD38, tac, Fc receptor, carcinoembryonic antigen (CEA), EpCAM, GpIIb/IIIa, RSV, CMV, HIV, Hep B, αvβ3, IL-17A, IL-17A/F, IL-17F, GD3 ganglioside; and human leukocyte antigen (HLA).

In yet another embodiment, at least monoclonal antibody binds to HER2.

In yet another embodiment, at least two monoclonal antibodies bind to HER2.

In a different embodiment, the IV bag contains a mixture of Trastuzumab and Pertuzumab.

In a further embodiment, at least one monoclonal antibody binds to CMV.

In a still further embodiment, at least two monoclonal antibodies bind to CMV.

In another embodiment, at least one monoclonal antibody binds to HCMV Complex I.

In yet another embodiment, at least one monoclonal antibody binds to HCMV gH.

In an additional embodiment, the IV bag contains a mixture of an antibody specifically binding to HCMV gH and an antibody specifically binding to HCMV Complex I.

All embodiments specifically listed herein, or otherwise disclosed in the specification, can be combined with each other in any combination. All such combinations are permutations and expressly within the scope of the invention herein.

### BRIEF DESCRIPTION OF THE DRAWINGS

FIG. 1 provides a schematic of the HER2 protein structure, and amino acid sequences for Domains I-IV (SEQ ID Nos.1-4, respectively) of the extracellular domain thereof.

FIGS. 2A and 2B depict alignments of the amino acid sequences of the variable light (V_{L}) (FIG. 2A) and variable heavy (V_{H}) (FIG. 2B) domains of murine monoclonal antibody 2C4 (SEQ ID Nos. 5 and 6, respectively); V_{L} and V_{H} domains of variant 574/Pertuzumab (SEQ ID Nos. 7 and 8, respectively), and human V_{L} and V_{H} consensus frameworks (hum κ1, light kappa subgroup I; humIII, heavy subgroup III) (SEQ ID Nos. 9 and 10, respectively). Asterisks identify differences between variable domains of Pertuzumab and murine monoclonal antibody 2C4 or between variable domains of Pertuzumab and the human framework. Complementarity Determining Regions (CDRs) are in brackets.

FIGS. 3A and 3B show the amino acid sequences of Pertuzumab light chain (FIG. 3A; SEQ ID NO. 11) and heavy chain (FIG. 3B; SEQ ID No. 12). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIGS. 4A and 4B show the amino acid sequences of Trastuzumab light chain (FIG. 4A; SEQ ID NO. 13) and heavy chain (FIG. 4B; SEQ ID NO. 14), respectively. Boundaries of the variable light and variable heavy domains are indicated by arrows.

FIGS. 5A and 5B depict a variant Pertuzumab light chain sequence (FIG. 5A; SEQ ID NO. 15) and a variant Pertuzumab heavy chain sequence (FIG. 5B; SEQ ID NO. 16), respectively.

FIG. 6 depicts Pertuzumab SEC profile of Pertuzumab/Trastuzumab mixture (840 mg) at 30° C. in 0.9% saline PO IV infusion bags (1) Time=0; (2) Time=24 hrs. Expanded view; full view (inset).

FIG. 7 shows Trastuzumab SEC profile of Pertuzumab/Trastuzumab mixture (840 mg) at 30° C. in 0.9% saline PO IV infusion bags (1) Time=0; (2) Time 24 hrs. Expanded view; full view (inset).

FIG. 8 shows Pertuzumab IEC profile of Pertuzumab/ Trastuzumab mixture at 30° C. in 0,9% saline PO IV infusion bags (1) Time=0; (2) Time 24 hrs. Full view.

FIG. 9 depicts Trastuzumab IEC profile of Pertuzumab/Trastuzumab mixture at 30° C. in 0.9% saline PO IV infusion bags (1) Time=0; (2) Time=24 hrs. Expanded view; Full view (inset).

FIG. 10 depicts CE-SDS LIF non-reduced profile of Pertuzumab/Trastuzumab mixture at 30° C. in 0.9% saline PO IV infusion bags (1) Time=0; (2) Time=24 hrs. Expanded view.

FIG. 11 shows CE-SDS LIF reduced profile of Pertuzumab/Trastuzumab mixture at 30° C. in 0.9% saline PO IV infusion bags (1) Time=0; (2) Time=24 hrs. Expanded view.

FIG. 12 is CZE (Q12417) of Pertuzumab/Trastuzumab mixture at 30° C. in 0.9% saline PO IV infusion bags (1) Time 0; (2) Time=24 hrs. Full view.

FIG. 13 shows iCIEF of Pertuzumab/Trastuzumab mixture at 30° C. in 0.9% saline PO IV infusion bags (1) Time=0; (2) Time=24 hours. Full view.

FIG. 14 shows potency dose response curves (xg/mL versus RFU) of Pertuzumab/Trastuzumab mixture, Pertuzumab alone, and Trastuzumab alone in 0.9% saline PO IV infusion bags (1) Time=0; (2) Time=24 hours.

FIG. 15 depicts Pertuzumab SEC profile of Pertuzumab/Trastuzumab mixture (1560 mg) in 0.9% saline IV infusion bags (1) PO 5° C. T0; (2) PO 5° C. T24 hrs; (3) PO 30° C. T0; (4) PO 30° C. T24 hrs; (5) PVC 5° C. T0; (6) PVC 5° C. T24 hrs; (7) PVC 30° C. T0; (8) PVC 30° C. T24 hrs. Expanded view; full view (inset).

FIG. 16 shows Trastuzumab SEC profile of Pertuzumab/Trastuzumab mixture (1560 mg) in 0.9% saline IV infusion bags (1) PO 5° C. T0; (2) PO 5° C. T24 hrs; (3) PO 30° C. T0; (4) PO 30° C. T24 hrs; (5) PVC 5° C. T0; (6) PVC 5° C. T24 hrs; (7) PVC 30° C. T0; (8) PVC 30° C. T24 hrs. Expanded view; full view (inset).

FIG. 17 shows Pertuzumab IEC (Pertuzumab-fast) profile of Pertuzumab/Trastuzumab mixture (1560 mg) in 0.9% saline IV infusion bags (1) PO 5° C. T0; (2) PO 5° C. T24 hrs; (3) PO 30° C. T0; (4) PO 30° C. T24 hrs; (5) PVC 5° C. T0; (6) PVC 5° C. T24 hrs; (7) PVC 30° C. T0; (8) PVC 30° C. T24 hrs. Full view.

FIG. 18 shows Trastuzumab IEC profile of Pertuzumab/Trastuzumab mixture (1560 mg) in 0.9% saline IV infusion bags (1) PO 5° C. T0; (2) PO 5° C. T24 hrs; (3) PO 30° C. T0; (4) PO 30° C. T24 hrs; (5) PVC 5° C. T0; (6) PVC 5° C. T24 hrs; (7) PVC 30° C. T0; (8) PVC 30° C. T24 hrs. Full view.

FIG. 19 depicts the study schema for Example 2.

FIG. 20 shows an amino acid sequence alignment of the heavy chain variable region (VH) of murine mAb 8G8 (SEQ ID NO:17) with selected human heavy chain variable region: VH1 FW (SEQ ID NO:18), human VH3 FW (SEQ ID NO:19), and human VH7 FW (SEQ ID NO:20). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 21 shows an amino acid sequence alignment of the light chain variable region (VL) of murine mAb 8G8 (SEQ ID NO:21) with human light chain variable region: λ3 FW region (SEQ ID NO:22) and human λ4 FW region (SEQ ID NO:23). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 22 shows mutant sequences in 8G8 HVR-L2. Shown are amino acid sequences of HVR-L2 and the first amino acid of FR3 (WT, SEQ ID NO:24; A1, SEQ ID NO:25; E1, SEQ ID NO:26; T1, SEQ ID NO:27; A2, SEQ ID NO:28; E2, SEQ ID NO:29; T2, SEQ ID NO:30; SG, SEQ ID NO:31; SGSG, SEQ ID NO:32; TGDA, SEQ ID NO:33). The numbers in the figure are based on Kabat numbering.

FIG. 23 shows an amino acid sequence alignment of the light chain variable region of murine mAb 8G8 (SEQ ID NO:21) with human light chain variable region λ4 FW (SEQ ID NO:23) and humanized light chain variable region for 8G8 on λ4 FW (hu8G8.λ4 FW) (SEQ ID NO:34). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 24 shows an amino acid sequence alignment of the heavy chain variable region of murine mAb 8G8 (SEQ ID NO:17) with human heavy chain variable regionVH1 Framework (VH1 FW) (SEQ ID NO:18) and the humanized heavy chain variable region for 8G8 on VH1 FW (hu8G8,VH1) (SEQ ID NO:35). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169). An exemplary nucleic acid sequence encoding for hu8G8.VH1 is also shown (SEQ ID NO:36).

FIG. 25 shows an amino acid sequence alignment of the heavy chain variable region of murine mAb 8G8 (SEQ ID NO:17) with human heavy chain variable region VH3 FW (SEQ ID NO:19) and the humanized heavy chain variable region of 8G8 on VH3 FW (hu8G8.VH3) (SEQ ID NO:37). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 26 shows an amino acid sequence alignment of the light chain variable region of murine mAb 8G8 V_{L} (SEQ ID NO:21) with the light chain variable region of λ4 FW region (SEQ ID NO:23) and the humanized light chain variable region of 8G8 on λ4 FW 8G8 graft) in which amino acid changes were introduced at amino acids 2 and 36 according to Kabat numbering (SEQ ID NO:38). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169). An exemplary nucleic acid sequence encoding for λ4 8G8 graft is also shown (SEQ ID NO:39). An exemplary nucleic acid sequence encoding residues 100-112 of λ4 FW (SEQ ID NO: 23) is also disclosed as SEQ ID NO: 117).

FIG. 27: shows an amino acid sequence alignment of human antibody MSL-109 with mAb HB1. Panel A: An alignment of MSL-109 VL (SEQ ID NO:40) with affinity-matured HB 1 VL (also SEQ ID NO:40 (100% identity)); and Panel B: an amino acid sequence alignment of human antibody MSL-109 VH (SEQ ID NO:41) with affinity-matured HB1 VH (SEQ ID NO:42). The amino acids are numbered according to Kabat numbering. The hypervariable regions (HVRs) are boxed.

FIG. 28: shows amino acid sequences of HVR-H2 from MSL-109 (SEQ ID NO:43) and IGHV3-21*01 (SEQ ID NO:44) and various amino acid substitutions made.

### DETAILED DESCRIPTION OF THE PREFERRED EMBODIMENTS

Glossary of abbreviations used herein: adverse drug reaction (ADR), adverse event (AE), alkaline phosphatase (ALP), absolute neutrophil count (ANC), area under the concentration-time curve (AUC), capillary zone electrophoresis (CZE), color, appearance and clarity (CAC), CLinical Evaluation Of Pertuzumab And TRAstuzumab (CLEOPATRA), confidence interval (CI), chromogenic in situ hybridization (CISH), maximum concentration (Cₘₐₓ), complete response (CR), case report form (CRF), computed tomography (CT), common terminology criteria for adverse events (CTCAE), Docetaxel (D), dose limiting toxicity (DLT), ethics committee (EC), epirubicin, cisplatin, and 5-fluorouracil (ECF), echocardiogram (ECHO), epidermal growth factor receptor (EGFR), estrogen receptor (ER), 5-fluorouracil, methotrexate, and doxorubicin (FAMTX), fluorescence in situ hybridization (FISH), 5-fluorouracil (5-FU), hazard ratio (HR), human epidermal growth factor receptor (EGFR), good clinical practice (GCP), human epidermal growth factor receptor 2 (HER2), ion exchange chromatography (IEC), immunohistochemistry (IHC), independent review facility (IRF), institutional review board (IRB), in situ hybridization (ISH), intravenous (IV), image capillary isoelectric focusing (iCIEF), left ventricular ejection fraction (LVEF), mitomycin C, cisplatin, and 5-fluorouracil (MCF), magnetic resonance image (MRI), multiple-gated acquisition (MUGA), not significant (NS), overall survival (OS), pathological complete response (pCR), polyolefin (PO), polyvinyl chloride (PVC), progressive disease (PD), progression free survival (PFS), pharmacokinetic (PK), partial response (PR), progesterone receptor (PgR), response evaluation criteria in solid tumors (RECIST), serious adverse event (SAE), size exclusion chromatography (SEC), stable disease (SD), study management team (SMT), sterile water for injection (SWFI), time to maximum plasma concentration (tₘₐₓ), upper limit of normal (ULN).

I. Definitions

The term “antibody-like molecule” is used herein to refer to any molecule that has an antigen binding region, and includes, without limitation, antibody fragments such as Fab′, Fab, F(ab′)₂, single domain antibodies (DABs), Fv, scFv (single chain Fv), affibodies, aptamers, immunoconjugates, immunoadhesins, and the like

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin, such as polypropylene (PP), or polyvinyl chloride (PVC).

A drug that is administered “concurrently” with one or more other drugs is administered during the same treatment cycle, on the same day of treatment as the one or more other drugs, and, optionally, at the same time as the one or more other drugs. For instance, for cancer therapies given every 3-weeks, the concurrently administered drugs are each administered on day-1 of a 3-week cycle.

By “co-administering” is meant intravenously administering two or more drugs during the same administration, rather than sequential infusions of the two or more drugs. Generally, this will involve combining the two or more drugs into the same IV bag prior to co-administration thereof.

By “stable mixture” when referring to a mixture of two or more drugs, such as Pertuzumab and Trastuzumab” means that each of the drugs in the mixture essentially retains its physical and chemical stability in the mixture as evaluated by one or more analytical assays. Exemplary analytical assays for this purpose include: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay. In one embodiment, mixture has been shown to be stable for up to about 8 hours, or up to about 12 hours, or up to about 24 hours at 5° C. or 30° C. In another embodiment, the mixture has been shown to be stable for at least about 8 hours, or at least about 12 hours, or at least about 24 hours at 5° C. or 30° C.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized HER2 antibodies specifically include Trastuzumab (HERCEPTIN®) as described in Table 3 of U.S. Pat. Co. 5,821,337 expressly incorporated herein by reference and as defined herein; and humanized 2C4 antibodies such as Pertuzumab as described and defined herein.

An “intact antibody” herein is one which comprises two antigen binding regions, and an Fc region. Preferably, the intact antibody has a functional Fc region. “Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

A “naked antibody” is an antibody that is not conjugated to a heterologous molecule, such as a cytotoxic moiety or radiolabel,

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “deamidated” antibody is one in which one or more asparagine residues thereof has been derivitized, e.g. to an aspartic acid, a succinimide, or an iso-aspartic acid.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “chimeric” antibody refers to an antibody in which a portion of the heavy and/or light chain is derived from a particular source or species, while the remainder of the heavy and/or light chain is derived from a different source or species.

A “HER receptor” is a receptor protein tyrosine kinase which belongs to the HER receptor family and includes EGFR, HER2, HERS and HERO receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is native sequence human HER receptor.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

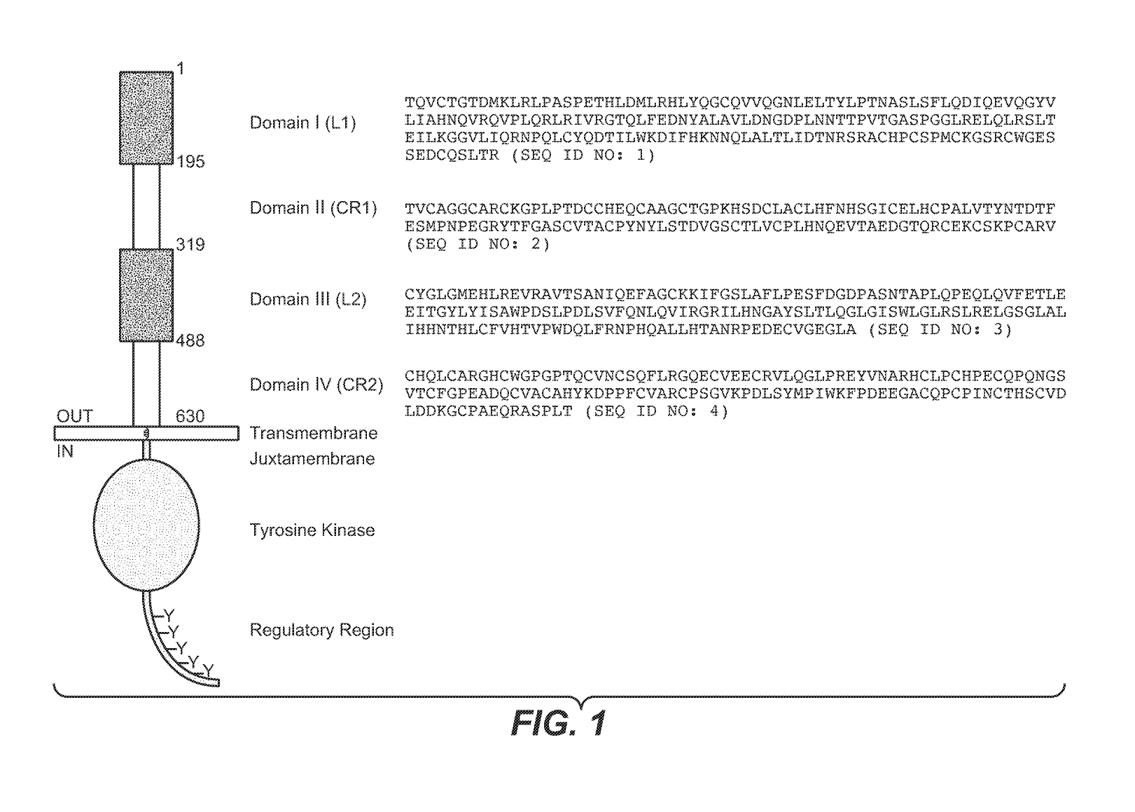

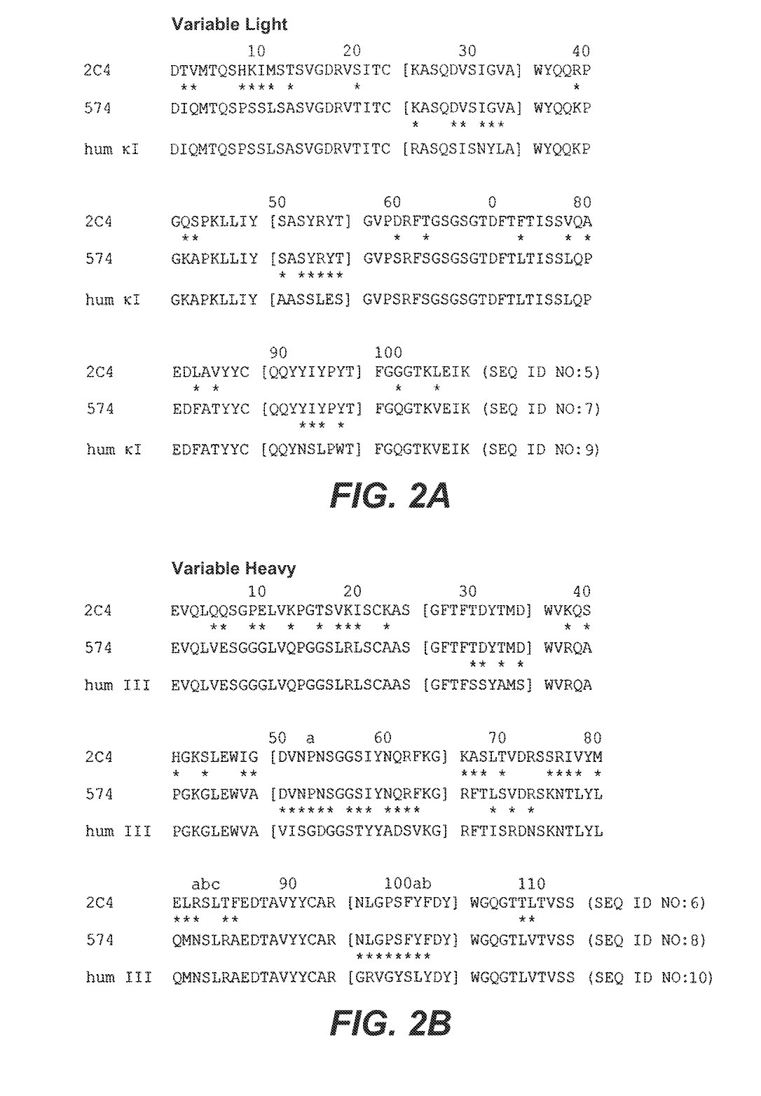

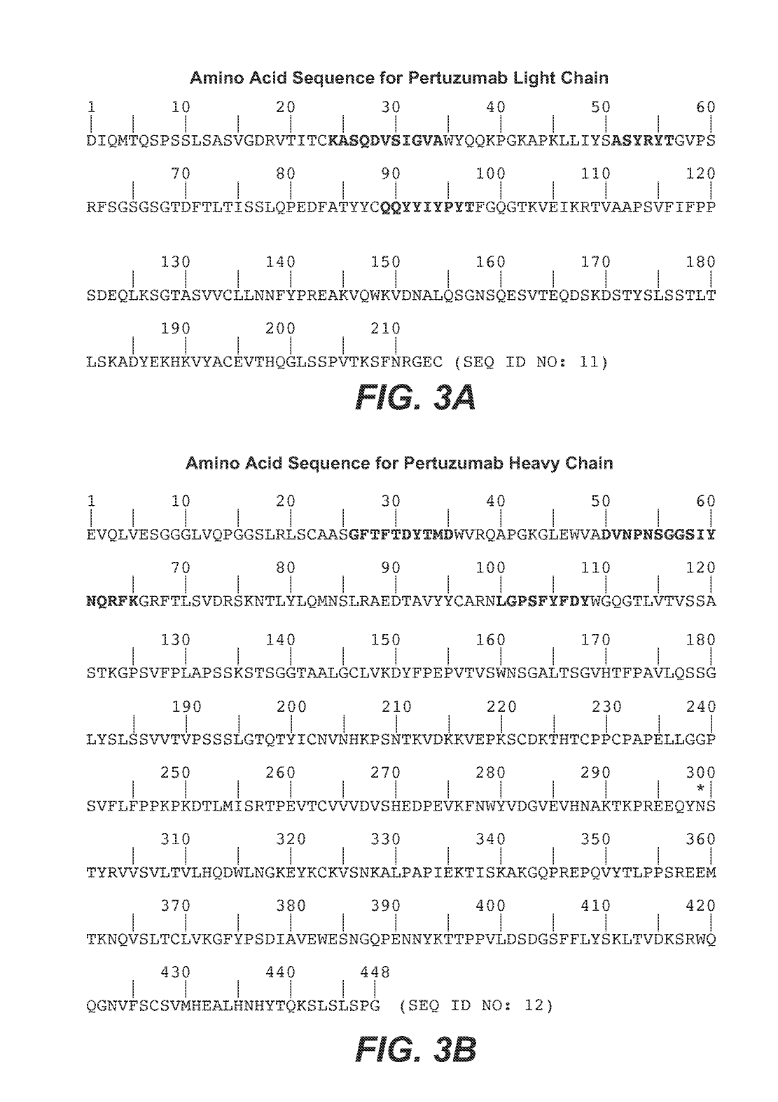

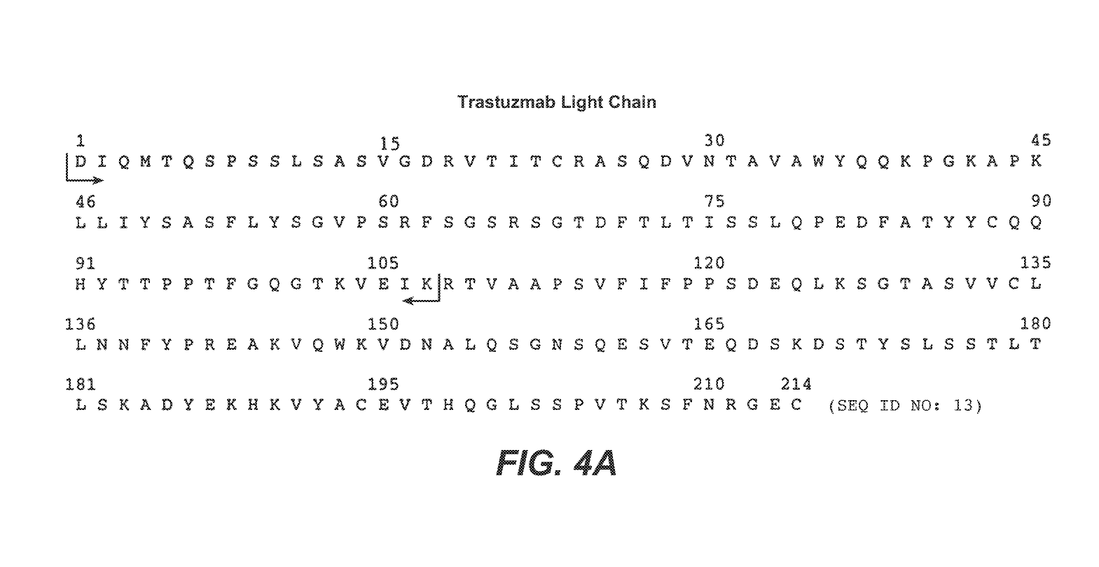

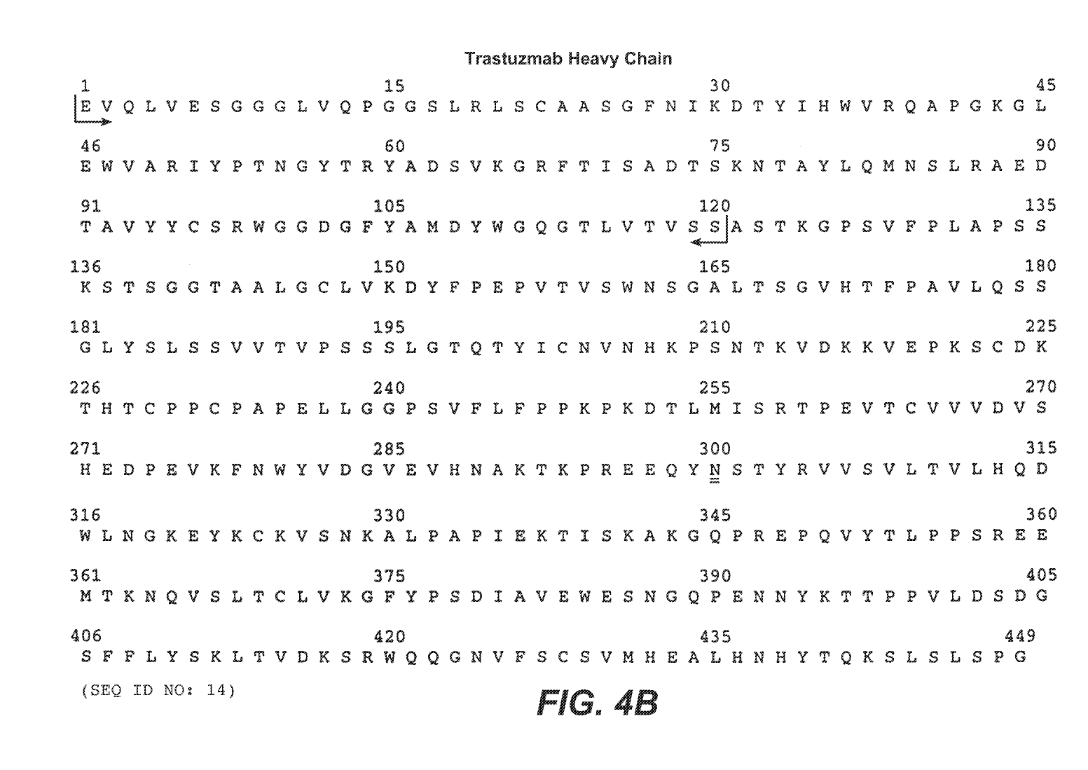

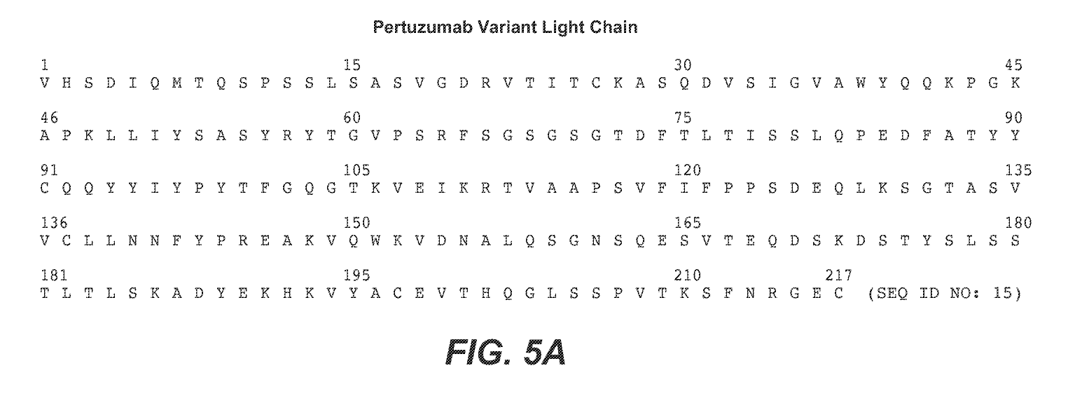

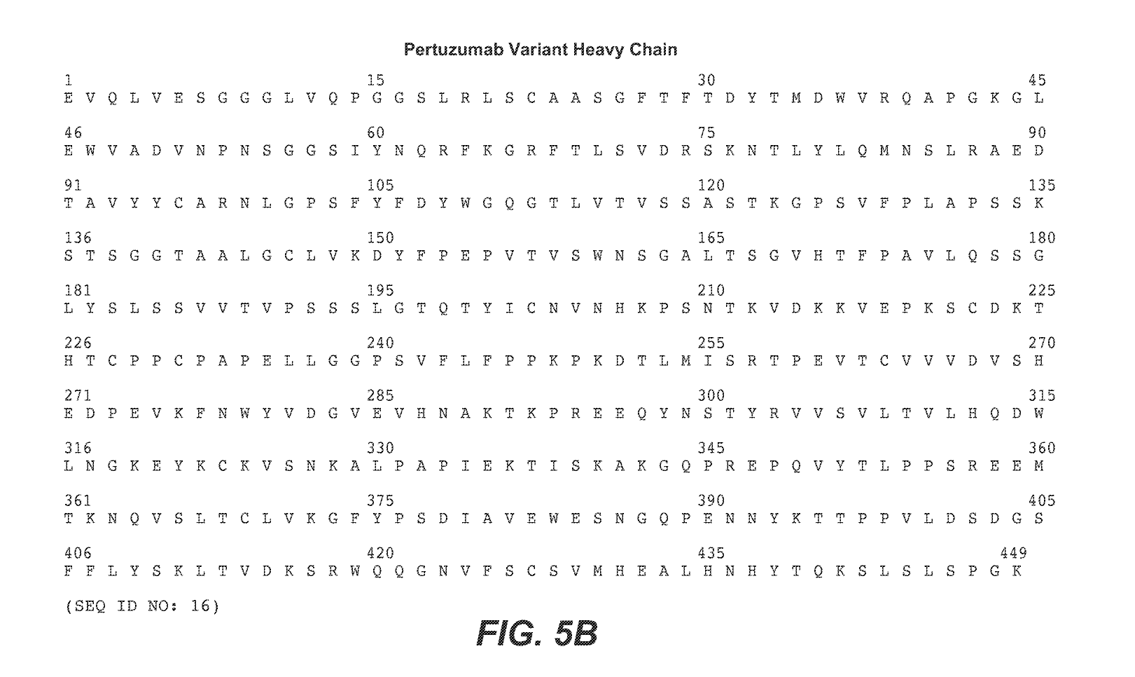

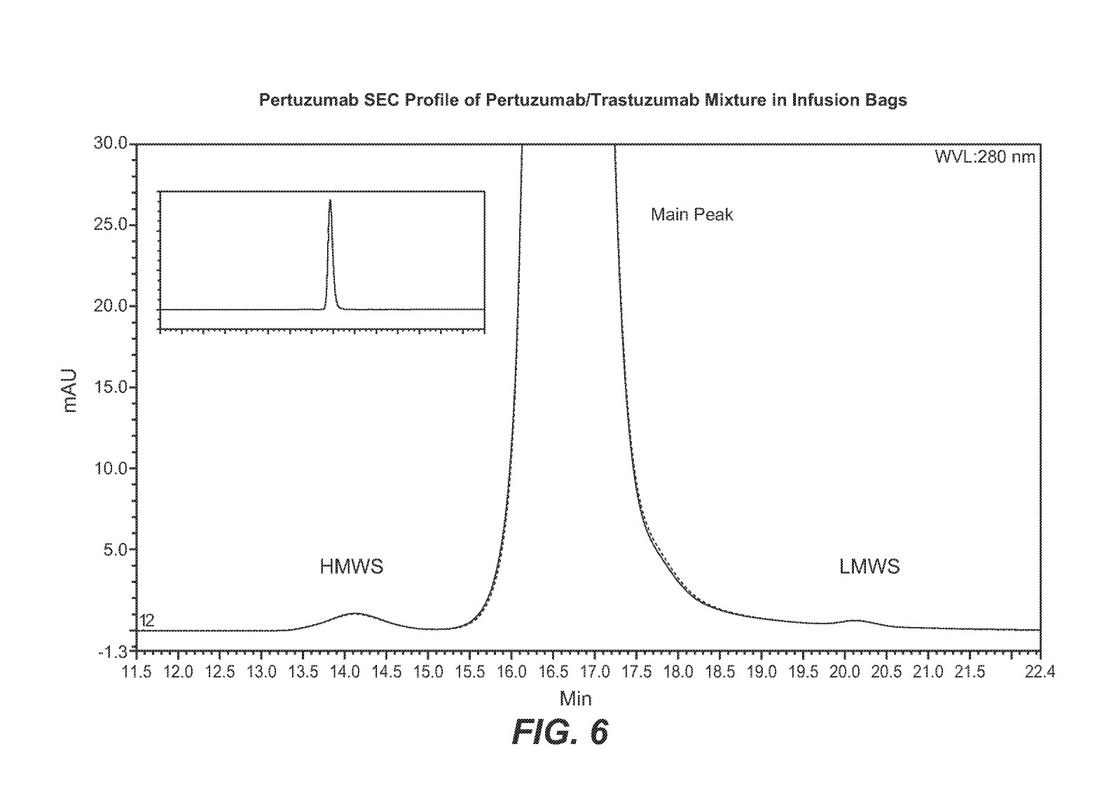

Herein, “HER2 extracellular domain” or “HER2 ECD” refers to a domain of HER2 that is outside of a cell, either anchored to a cell membrane, or in circulation, including fragments thereof. The amino acid sequence of HER2 is shown in FIG. 1. In one embodiment, the extracellular domain of HER2 may comprise four domains: “Domain I” (amino acid residues from about 1-195; SEQ ID NO:1), “Domain II” (amino acid residues from about 196-319; SEQ ID NO:2), “Domain III” (amino acid residues from about 320-488: SEQ ID NO:3), and “Domain IV” (amino acid residues from about 489-630; SEQ ID NO:4) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 6 herein.

A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Other proteins, such as a cytokine receptor subunit (e.g. gp130) may be associated with the dimer. Preferably, the HER dimer comprises HER2.

A “HER heterodimer” herein is a noncovalently associated heterodimer comprising at least two different HER receptors, such as EGFR-HER2, HER2-HER3 or HER2-HERO heterodimers.

A “HER antibody” is an antibody that binds to a HER receptor. Optionally, the HER antibody further interferes with HER activation or function. Preferably, the HER antibody binds to the HER2 receptor. HER2 antibodies of interest herein are Pertuzumab and Trastuzumab.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

An antibody which “inhibits HER dimerization” is an antibody which inhibits, or interferes with, formation of a HER dimer. Preferably, such an antibody binds to HER2 at the heterodimeric binding site thereof. The most preferred dimerization inhibiting antibody herein is Pertuzumab or MAb 2C4. Other examples of antibodies which inhibit HER dimerization include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; and antibodies which bind to HER4 and inhibit dimerization thereof with one or more other HER receptors.

A “HER2 dimerization inhibitor” is an agent that inhibits formation of a dimer or heterodimer comprising HER2.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2 (SEQ ID NO: 15). Franklin et al. Cancer Cell 5:317-328 (2004).

A HER2 antibody that “binds to a heterodimeric binding site” of HER2, binds to residues in Domain II (SEQ ID NO: 2) and optionally also binds to residues in other of the domains of the HER2 extracellular domain, such as domains I and III, SEQ ID NOs: 1 and 3), and can sterically hinder, at least to some extent, formation of a HER2-EGFR, HER2-HER3, or HER2-HER4 heterodimer. Franklin et al, Cancer Cell 5:317-328 (2004) characterize the HER2-Pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II (SEQ ID NO: 2) and optionally residues in other domain(s) of HER2, such as domains I and III (SEQ ID NOs: 1 and 3, respectively). Preferably the antibody that binds to domain II binds to the junction between domains I, II and III of HER2.

For the purposes herein, “Pertuzumab” and “rhuMAb 2C4”, which are used interchangeably, refer to an antibody comprising the variable light and variable heavy amino acid sequences in SEQ ID NOs: 7 and 8, respectively. Where Pertuzumab is an intact antibody, it preferably comprises an IgG1 antibody; in one embodiment comprising the light chain amino acid sequence in SEQ ID NO: 11 or 15, and heavy chain amino acid sequence in SEQ ID NO: 12 or 16. The antibody is optionally produced by recombinant Chinese Hamster Ovary (CHO) cells. The terms “Pertuzumab” and “rhuMAb 2C4” herein cover biosimilar versions of the drug with the United States Adopted Name (USAN) or International Nonproprietary Name (INN): Pertuzumab.

For the purposes herein, “Trastuzumab” and rhuMAb4D5”, which are used interchangeably, refer to an antibody comprising the variable light and variable heavy amino acid sequences from within SEQ ID Nos: 13 and 14, respectively. Where Trastuzumab is an intact antibody, it preferably comprises an IgG1 antibody; in one embodiment comprising the light chain amino acid sequence of SEQ ID NO: 13 and the heavy chain amino acid sequence of SEQ ID NO: 14. The antibody is optionally produced by Chinese Hamster Ovary (CHO) cells. The terms “Trastuzumab” and “rhuMAb4D5” herein cover biosimilar versions of the drug with the United States Adopted Name (USAN) or International Nonproprietary Name (INN): Trastuzumab.

The term “Complex I,” as used herein, refers to any native Complex I from any cytomegalovirus source, including CMV that infects mammals such as primates (e.g., humans) and rodents (e.g., mice and rats), unless otherwise indicated. The term encompasses a combination of all of gH, gL, UL128, UL130 and UL131 polypeptides. The term also encompasses naturally occurring variants of the proteins of Complex I, e.g., splice variants or allelic variants. The amino acid sequence of an exemplary HCMV gH is shown in SEQ ID NO:45. The amino acid sequence of an exemplary HCMV gL is shown in SEQ ID NO:46. The amino acid sequence of an exemplary HCMV UL128 is shown in SEQ ID NO:47. The amino acid sequence of an exemplary HCMV UL130 is shown in SEQ ID NO:48. The amino acid sequence of an exemplary HCMV UL131 is shown in SEQ ID NO:49. Additional exemplary sequences for HCMV gH, gL, UL128, UL130 and UL131 may be found in Genbank Accession number GU179289 (Dargan et al., J. Gen. Virol. 91: 1535-1546 (2010)), which are both incorporated by reference herein in their entireties, and are included herein as SEQ ID NO: 51 (gH), SEQ ID NO: 52 (gL), SEQ ID NO: 96 (UL128), SEQ ID NO: 94 (UL130); and SEQ ID NO: 97 (UL131).

The term “Complex II,” as used herein, refers to any native Complex II from any cytomegalovirus source, including CMV that infects mammals such as primates (e.g., humans) and rodents (e.g., mice and rats), unless otherwise indicated. The term encompasses a combination of all of gH, gL and gO. The term also encompasses naturally occurring variants of the proteins of Complex II, e.g., splice variants or allelic variants. The amino acid sequence of an exemplary HCMV gH is shown in SEQ ID NO:45. The amino acid sequence of an exemplary HCMV gL is shown in SEQ ID NO:46. The amino acid sequence of an exemplary HCMV gO is shown in SEQ ID NO:50. Additional exemplary sequences for HCMV gH, gL and gO may be found in Genbank Accession number GU179289 (Dargan et al., J. Gen. Virol. 91: 1535-1546 (2010)), which are both incorporated by reference herein in their entireties, and are included herein as SEQ ID NO: 51 (gH), SEQ ID NO: 52 (gL) and SEQ ID NO: 53 (gO).

The term “gH,” as used herein, refers to any native gH from any vertebrate source, including mammals such as primates (e.g. humans) and rodents (e.g., mice and rats), unless otherwise indicated. The term encompasses “full-length,” unprocessed gH as well as any form of gH that results from processing in the cell. The term also encompasses naturally occurring variants of gH, e.g., splice variants or allelic variants. The amino acid sequence of gH is about 95% identical among CMV isolates. The amino acid sequence of an exemplary HCMV gH is shown in SEQ ID NO:45. An additional exemplary sequence for HCMV gH may be found in Genbank Accession number GU179289 (Dargan et al., J. Gen. Virol. 91: 1535-1546 (2010)), which are both incorporated by reference herein in their entireties, and is included herein as SEQ ID NO: 51 (gH).

The terms “anti-Complex I antibody” and “an antibody that binds to Complex I” refer to an antibody that is capable of binding Complex I with sufficient affinity such that the antibody is useful as a diagnostic and/or therapeutic agent in targeting Complex I. In one embodiment, the extent of binding of an anti-Complex I antibody to an unrelated, non-Complex I protein is less than about 10% of the binding of the antibody to Complex I as measured, e.g., by a radioimmunoassay (RIA). In certain embodiments, an antibody that binds to Complex I has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³M, e.g., from 10⁻⁹ M to 10⁻¹³M). In certain embodiments, an anti-Complex I antibody binds to an epitope of Complex I that is conserved among human CMV isolates. In certain embodiments, an anti-Complex I antibody binds to an epitope of Complex I that is conserved among CMV strains that infect different species. In certain embodiments, the “anti-Complex I antibody” binds a conformational epitope of Complex I and in certain embodiments the anti-Complex I antibody binds to an epitope within an individual protein member of Complex I which is not gH (i.e., gL, UL128, UL130 or UL131).

The terms “anti-gH antibody” and “an antibody that binds to gH” refer to an antibody that is capable of binding gH with sufficient affinity such that the antibody is useful as a diagnostic and/or therapeutic agent in targeting gH. In one embodiment, the extent of binding of an anti-gH antibody to an unrelated, non-gH protein is less than about 10% of the binding of the antibody to gH as measured, e.g., by a radioimmunoassay (RIA). In certain embodiments, an antibody that binds to gH has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³M). In certain embodiments, an anti-gH antibody binds to an epitope of gH that is conserved among human CMV isolates. In certain embodiments, an anti-gH antibody binds to an epitope of gH that is conserved among CMV strains that infect different species.

The term “VEGF” as used herein refers to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. According to a preferred embodiment, the VEGF is a human VEGF.

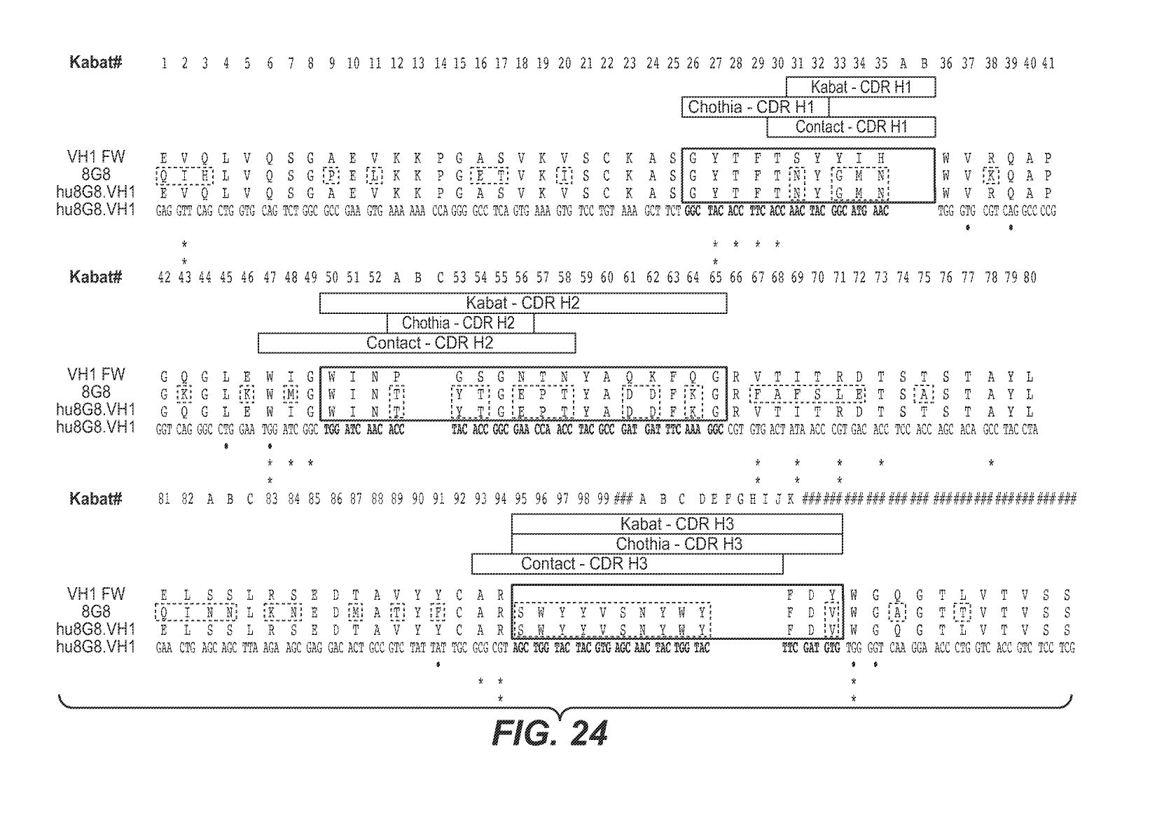

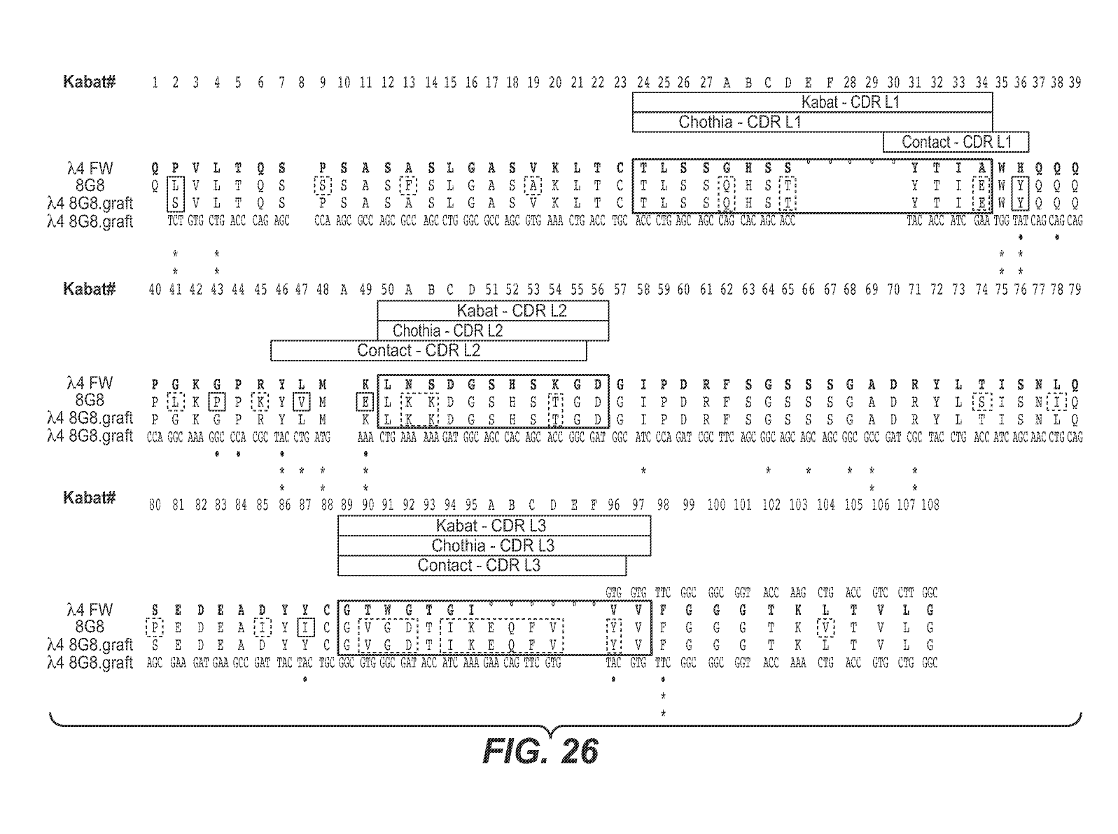

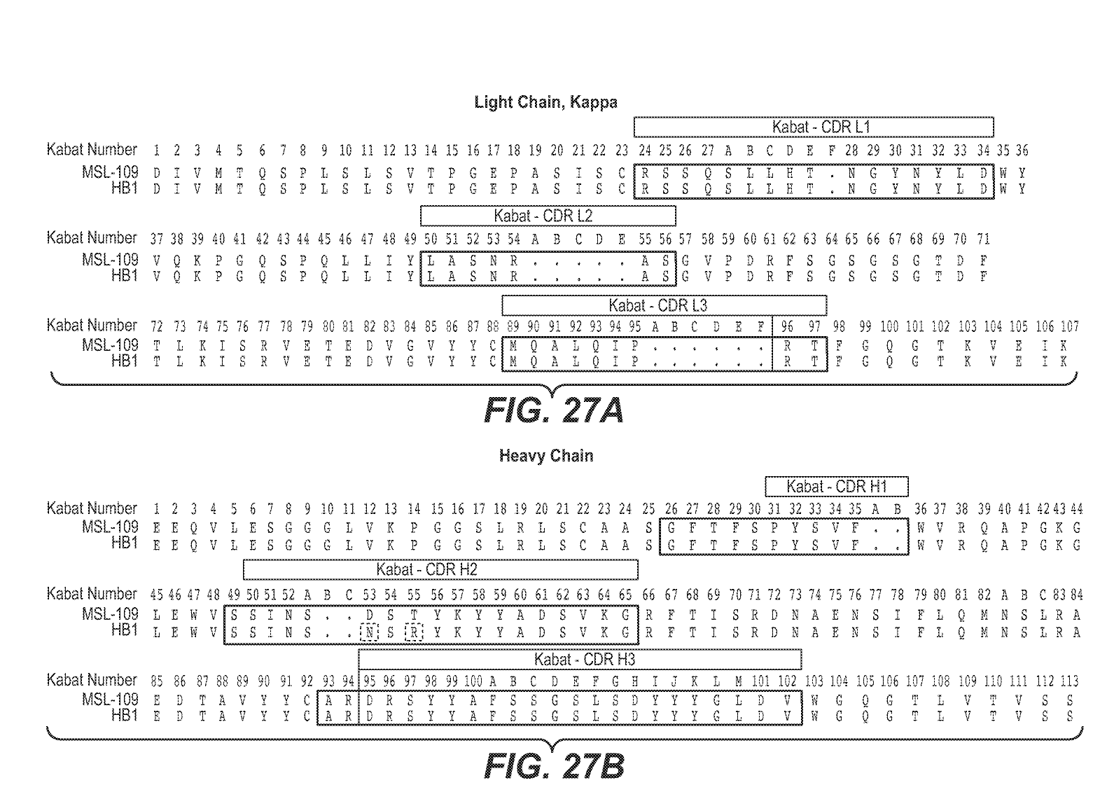

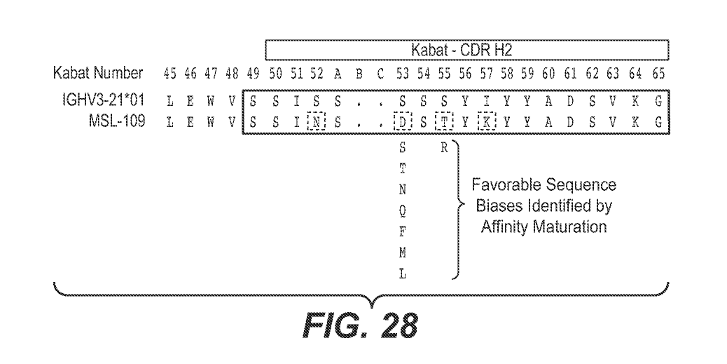

An “anti-VEGF antibody” is an antibody that binds to VEGF with sufficient affinity and specificity. Preferably, the anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as P1GF, PDGF or bFGF. A preferred anti-VEGF antibody is a monoclonal antibody that binds to the same epitope as the monoclonal anti-VEGF antibody A4.6.1 produced by hybridoma ATCC HB 10709. More preferably the anti-VEGF antibody is a recombinant humanized anti-VEGF monoclonal antibody generated according to Presta et al. (1997) Cancer Res. 57:4593-4599, including but not limited to the antibody known as bevacizumab (BV; Avastin®). According to another embodiment, anti-VEGF antibodies that can be used include, but are not limited to the antibodies disclosed in WO 2005/012359. According to one embodiment, the anti-VEGF antibody comprises the variable heavy and variable light region of any one of the antibodies disclosed in FIGS. 24, 25, 26, 27 and 29 of WO 2005/012359 (e.g., G6, G6-23, G6-31, G6-23.1, G6-23.2, B20, B20-4 and B20.4.1). In another preferred embodiment, the anti-VEGF antibody known as ranibizumab is the VEGF antagonist administered for ocular disease such as diabetic neuropathy and AMD.

The anti-VEGF antibody “Bevacizumab (BV)”, also known as “rhuMAb VEGF” or “Avastin®”, is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Other anti-VEGF antibodies include the antibodies described in U.S. Pat. No. 6,884,879 and WO 2005/044853.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Herein, a “patient” is a human patient. The patient may be a “cancer patient,” i.e. one who is suffering or at risk for suffering from one or more symptoms of cancer, such as, for example, gastric or breast cancer.

A “patient population” refers to a group of patients, such as cancer patients. Such populations can be used to demonstrate statistically significant efficacy and/or safety of a drug.

A “relapsed” patient is one who has signs or symptoms of a disease or pathological condition, such as cancer, after remission. Optionally, the patient has relapsed after adjuvant or neoadjuvant therapy.

An “infant” as used herein, refers to an individual or subject ranging in age from birth to not more than about one year and includes infants from 0 to about 12 months.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative. As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions of the invention are used to delay development of a disease or to slow the progression of a disease or to decrease incidence of a disease or the severity of disease symptoms.

The term “chemotherapy” as used herein refers to treatment comprising the administration of a chemotherapy, as defined hereinbelow.

“Survival” refers to the patient remaining alive, and includes overall survival as well as progression free survival.

“Overall survival” or “OS” refers to the patient remaining alive for a defined period of time, such as 1 year, 5 years, etc from the time of diagnosis or treatment. For the purposes of the clinical trial described in the example, overall survival (OS) is defined as the time from the date of randomization of patient population to the date of death from any cause.

“Progression free survival” or “PFS” refers to the patient remaining alive, without the cancer progressing or getting worse. For the purpose of the clinical trial described in the example, progression free survival (PFS) is defined as the time from randomization of study population to the first documented progressive disease, or unmanageable toxicity, or death from any cause, whichever occurs first. Disease progression can be documented by any clinically accepted methods, such as, for example, radiographical progressive disease, as determined by Response Evaluation Criteria in Solid Tumors (RECIST) (Therasse et al., J Natl Ca. Inst 2000; 92(3):205-216), carcinomatous meningitis diagnosed by cytologic evaluation of cerebral spinal fluid, and/or medical photography to monitor chest wall recurrences of subcutaneous lesions.

By “extending survival” is meant increasing overall or progression free survival in a patient treated in accordance with the present invention relative to an untreated patient and/or relative to a patient treated with one or more approved anti-tumor agents, but not receiving treatment in accordance with the present invention. In a particular example, “extending survival” means extending progression-free survival (PFS) and/or overall survival (OS) of cancer patients receiving the combination therapy of the present invention (e.g. treatment with a combination of Pertuzumab, Trastuzumab and a chemotherapy) relative to patients treated with Trastuzumab and the chemotherapy only. In another particular example, “extending survival” means extending progression-free survival (PFS) and/or overall survival (OS) of cancer patients receiving the combination therapy of the present invention (e.g. treatment with a combination of Pertuzumab, Trastuzumab and a chemotherapy) relative to patients treated with Pertuzumab and the chemotherapy only.

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The terms “cancer” and “cancerous” refer to or describe the physiological condition in mammals that is typically characterized by unregulated cell growth.

“Gastric cancer” specifically includes metastatic or locally advanced non-resectable gastric cancer, including, without limitation, histologically confirmed adenocarcinoma of the stomach or gastroesophageal junction with inoperable (non-resectable) locally advanced or metastatic disease, not amenable to curative therapy, and post-operatively recurrent advanced gastric cancer, such as adenocarcinoma of the stomach or gastroesophageal junction, when the intent of the surgery was to cure the disease.

An “advanced” cancer is one which has spread outside the site or organ of origin, either by local invasion or metastasis. Accordingly, the term “advanced” cancer includes both locally advanced and metastatic disease.

A “refractory” cancer is one which progresses even though an anti-tumor agent, such as a chemotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

A “non-resectable” or “unresectable” cancer is not able to be removed (resected) by surgery.

“Early-stage breast cancer” herein refers to breast cancer that has not spread beyond the breast or the axillary lymph nodes. Such cancer is generally treated with neoadjuvant or adjuvant therapy.

“Neoadjuvant therapy” refers to systemic therapy given prior to surgery.

“Adjuvant therapy” refers to systemic therapy given after surgery.

“Metastatic” cancer refers to cancer which has spread from one part of the body (e.g. the breast) to another part of the body.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer or biological sample which “displays HER activation” is one which, in a diagnostic test, demonstrates activation or phosphorylation of a HER receptor. Such activation can be determined directly (e.g. by measuring HER phosphorylation by ELISA) or indirectly (e.g. by gene expression profiling or by detecting HER heterodimers, as described herein).

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via in situ hybridization (ISH), including fluorescent in situ hybridization (FISH; see WO98/45479 published October, 1998) and chromogenic in situ hybridization (CISH; see, e.g. Tanner et al., Am. J. Pathol. 157(5): 1467-1472 (2000); Bella et al., J. Clin. Oncol. 26: (May 20 suppl; abstr 22147) (2008)), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A “HER2-positive” cancer comprises cancer cells which have higher than normal levels of HER2. Examples of HER2-positive cancer include HER2-positive breast cancer and HER2-positive gastric cancer. Optionally, HER2-positive cancer has an immunohistochemistry (IHC) score of 2+ or 3+ and/or an in situ. hybridization (ISH) amplification ratio ≧2.0.

Herein, an “anti-tumor agent” refers to a drug used to treat cancer. Non-limiting examples of anti-tumor agents herein include chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxene, dual tyrosine kinase inhibitors, TLK286, EMD-7200, Pertuzumab, Trastuzumab, erlotinib, and bevacizumab.

The “epitope 2C4” is the region in the extracellular domain of HER2 to which the antibody 2C4 binds. In order to screen for antibodies which bind essentially to the 2C4 epitope, a routine cross-blocking assay such as that described in Antibodies, A Laboratory Manual, Cold Spring Harbor Laboratory, Ed Harlow and David Lane (1988), can be performed. Preferably the antibody blocks 2C4's binding to HER2 by about 50% or more. Alternatively, epitope mapping can be performed to assess whether the antibody binds essentially to the 2C4 epitope of HER2. Epitope 2C4 comprises residues from Domain II (SEQ ID NO: 2) in the extracellular domain of HER2. 2C4 and Pertuzumab binds to the extracellular domain of HER2 at the junction of domains I, II and III (SEQ ID NOs: 1, 2, and 3, respectively). Franklin et al. Cancer Cell 5:317-328 (2004).

The “epitope 4D5” is the region in the extracellular domain of HER2 to which the antibody 4D5 (ATCC CRL 10463) and Trastuzumab bind. This epitope is close to the transmembrane domain of HER2, and within Domain IV of HER2 (SEQ ID NO: 4). To screen for antibodies which bind essentially to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds essentially to the 4D5 epitope of HER2 (e.g. any one or more residues in the region from about residue 529 to about residue 625, inclusive of the HER2 ECD, residue numbering including signal peptide).

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapy” is use of a chemical compound useful in the treatment of cancer. Examples of chemotherapeutic agents, used in chemotherapy, include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX™, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxanes; chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “taxane” is a chemotherapy which inhibits mitosis and interferes with microtubules. Examples of taxanes include Paclitaxel (TAXOL®; Bristol-Myers Squibb Oncology, Princeton, N.J.); cremophor-free, albumin-engineered nanoparticle formulation of paclitaxel (ABRAXANE™; American Pharmaceutical Partners, Schaumberg, Ill.); and Docetaxel (TAXOTERE®; Rhone-Poulenc Rorer, Antony, France).

An “anthracycline” is a type of antibiotic that comes from the fungus Streptococcus peucetius, examples include: Daunorubicin, Doxorubicin, and Epirubicin, etc.

“Anthracycline-based chemotherapy” refers to a chemotherapy regimen that consists of or include one or more anthracycline. Examples include 5-FU, epirubicin, and cyclophosphamide (FEC); 5-FU, doxorubicin, and cyclophosphamide (FAC); doxorubicin and cyclophosphamide (AC); epirubicin and cyclophosphamide (EC); etc.

For the purposes herein, “carboplatin-based chemotherapy” refers to a chemotherapy regimen that consists of or includes one or more Carboplatins. An example is TCH (Docetaxel/TAXOL®, Carboplatin, and Trastuzumab/HERCEPTIN®).

An “aromatase inhibitor” inhibits the enzyme aromatase, which regulates estrogen production in the adrenal glands. Examples of aromatase inhibitors include: 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In one embodiment,the aromatase inhibitor herein is letrozole or anastrozole.

An “antimetabolite chemotherapy” is use of an agent which is structurally similar to a metabolite, but cannot be used by the body in a productive manner. Many antimetabolite chemotherapy interferes with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

The term “platin” is used herein to refer to platinum based chemotherapy, including, without limitation, cisplatin, carboplatin, and oxaliplatin.

The term “fluoropyrimidine” is used herein to refer to an antimetabolite chemotherapy, including, without limitation, capecitabine, floxuridine, and fluorouracil (5-FU).

A “fixed ” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Cardiac toxicity” refers to any toxic side effect resulting from administration of a drug or drug combination. Cardiac toxicity can be evaluated based on any one or more of: incidence of symptomatic left ventricular systolic dysfunction (LVSD) or congestive heart failure (CHF), or decrease in left ventricular ejection fraction (LVEF).

The phrase “without increasing cardiac toxicity” for a drug combination including Pertuzumab refers to an incidence of cardiac toxicity that is equal or less than that observed in patients treated with drugs other than Pertuzumab in the drug combination (e.g. equal or less than that resulting from administration of Trastuzumab and the chemotherapy, e.g. Docetaxel).

A “vial” is a container suitable for holding a liquid or lyophilized preparation. In one embodiment, the vial is a single-use vial, e.g. a 20-cc single-use vial with a stopper.

A “package insert” is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

The expression “safety data” concerns the data obtained in a controlled clinical trial showing the prevalence and severity of adverse events to guide the user regarding the safety of the drug, including guidance on how to monitor and prevent adverse reactions to the drug. The safety data comprises any one or more (e.g. two, three, four or more) of the most common adverse events (AEs) or adverse reactions (ADRs).

“Efficacy data” refers to the data obtained in controlled clinical trial showing that a drug effectively treats a disease, such as cancer.

II. Co-Administration of at Least Two Antibodies or Antibody-Like Molecules From the Same Intravenous Infusion Bag

Intravenous infusion solutions are typically supplied in standard disposable plastic bags. The bag is suspended from a hook on a stand and the infusion liquid is delivered into the vein or bone marrow by an injection or trocar needle. Various designs of IV bags, including pressurized and non-pressurized bags, typically made of polyvinyl chloride (PVC) or a polyolefin, such as polypropylene (PP), are commercially available in a variety of sizes from a variety of manufacturers.

The present invention concerns an article of manufacture, such as an IV bag, which contains a stable liquid mixture of at least two monoclonal antibodies and/or antibody-like molecules, formulated separately, for co-administration to a subject, such as a patient, in need. The invention also concerns an intravenous infusion device comprising an IV bag, which contains a stable liquid mixture of at least two monoclonal antibodies and/or antibody-like molecules, formulated separately, for co-administration to a subject, such as a patient, in need.

The invention further concerns methods for co-administration of at least two antibodies and/or antibody-like molecules, formulated separately, by intravenous infusion from a single IV bag. If the treatment regimen includes a loading dose followed by one or more maintenance doses, co-administration of two or more monoclonal antibodies may be carried out, for example, during the maintenance dose phase, after the initial loading dose has been infused and monitored.

Co-administration of two or more antibodies and/or antibody-like molecules does not mean that the antibodies or antibody-like molecules are co-formulated. On the contrary, the co-administered antibodies and/or antibody-like molecules of the present invention are present in separate formulations prior to addition to the same IV bag. It is, however, possible that two or more of the co-administered antibodies or antibody-like molecules are present in formulations having identical compositions (but for the particular antibody or antibody-like molecule), prior to addition to the same IV bag.

Combining two or more antibodies and/or antibody-like molecules into one IV bag presents special challenges, including the compatibility of the antibodies/antibody-like molecules and the various components of the individual formulations, such as diluents.

(ii) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

U.S. Pat. No. 6,949,245 describes production of exemplary humanized HER2 antibodies which bind HER2 and block ligand activation of a HER receptor.

Humanized HER2 antibodies specifically include Trastuzumab (HERCEPTIN®) as described in Table 3 of U.S. Pat. No. 5,821,337 expressly incorporated herein by reference and as defined herein; and humanized 2C4 antibodies such as Pertuzumab as described and defined herein.

The humanized antibodies herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al., Sequences of Proteins of Immunological Interest, 5th Ed. Public Health Service, National Institutes of Health, Bethesda, Md. (1991). In one embodiment, the humanized antibody comprises FR substitutions at two or all of positions 69H, 71H and 73H.

(iii) Multispecific Antibodies

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein. Examples of BsAbs include those with one arm directed against a tumor cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; BsAbs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA); BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor:CD3 complex/anti-influenza, anti-FcγR/anti-HIV; BsAbs for tumor detection in vitro or in vivo such as anti-CEA/anti-EOTUBE, anti-CEA/anti-DPTA, anti- p185^{HER2}/anti-hapten; BsAbs as vaccine adjuvants; and BsAbs as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase. Examples of trispecific antibodies include anti-CD3/anti-CD4/anti-CD37, anti-CD3/anti-CD5/anti-CD37 and anti-CD3/anti-CD8/anti-CD37. Bispecific antibodies can be prepared as full length antibodies or antibody fragments (e.g. F(ab′)₂ bispecific antibodies). Bispecific antibodies are reviewed in Segal et al. J. Immunol. Methods 248:1-6 (2001).

Methods for making bispecific antibodies are known in the art. Traditional production of full length bispecific antibodies is based on the coexpression of two immunoglobulin heavy chain-light chain pairs, where the two chains have different specificities (Milstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/20373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies are contemplated. For example, trispecific antibodies can be prepared. Tutt et al. J. Immunol. 147: 60 (1991).

(iv) Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fe region, X1 and X2 represent an amino acid or polypeptide, and n is 0 or 1. For instance, the polypeptide chain(s) may comprise: VH—CH I -flexible linker-VH—CH1-Fc region chain; or VH—CH1-VH—CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. Multivalent antibodies are described in WO 01/00238 and WO 00/44788.

(v) Affinity Matured Antibodies

The antibody herein may be an affinity matured antibody comprising substitution(s) of one or more hypervariable region residues of a parent antibody (e.g. a humanized or human antibody).

Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity). In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and its antigen. Such contact residues and neighboring residues are candidates for substitution according to the techniques elaborated herein. Once such variants are generated, the panel of variants is subjected to screening, and antibodies with superior properties in one or more relevant assays may be selected for further development.

(vi) Immunoconjugates

Conjugates of an antibody and one or more small molecule toxins, such as a calicheamicin, maytansinoids, a trichothene, and CC1065, and the derivatives of these toxins that have toxin activity, are also contemplated herein.

In one preferred embodiment, the antibody, or antibody-like molecule, is conjugated to one or more maytansinoid molecules.

Antibody-maytansinoid conjugates may be prepared by chemically linking the antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pa. No. 5,208,020. Preferred maytansinoids are maytansinol and maytansinol analogues modified in the aromatic ring or at other positions of the maytansinol molecule, such as various maytansinol esters. There are many linking groups known in the art for making antibody-maytansinoid conjugates, including, for example, those disclosed in U.S. Pat. No. 5,208,020 or EP Patent 0 425 235 B1, and Chari et al. Cancer Research 52: 127-131 (1992). The linking groups include disufide groups, thioether groups, acid labile groups, photolabile groups, peptidase labile groups, or esterase labile groups, as disclosed in the above-identified patents, disulfide and thioether groups being preferred. Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio)pentanoate (SPP) to provide for a disulfide linkage. The linker may be attached to the maytansinoid molecule at various positions, depending on the type of the link For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another immunoconjugate of interest comprises the antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N acetyl γ₁^{I}, PSAG and θ^{I}₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the glycoprotein can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between the antibody and a compound with nucleolytic activity (e.g. a ribonuclease or a DNA endonuclease such as a deoxyribonuclease; DNase).

For selective destruction of a tumor, an anti-cancer antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al. Biochem. Biophys. Res. Commun. 80: 49-57 (1978) can be used to incorporate iodine-123. “Monoclonal Antibodies in Immunoscintigraphy” (Chatal,CRC Press 1989) describes other methods in detail.

Conjugates of the antibodies and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis-(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026, The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

Alternatively, a fusion protein comprising the antibody and cytotoxic agent may be made, e.g. by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

(vii) Exemplary Antigen Targets

Antigen targets for therapeutic antibodies and/antibody-like molecules include transmembrane molecules (e.g. receptors) and ligands such as growth factors. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; a tumor necrosis factor (TNF) such as TNF-α or TNF-β; insulin-like growth factor-I and -II (IGF-I and IGF-II); des(1-3)-IGF-I (brain IGF-I), insulin-like growth factor binding proteins; CD proteins such as CD3, CD4, CD8, CD19, CD20, CD22 and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1, IL-2, IL-3, IL-4, IL-5, IL-6, IL-7, IL-8, IL-9 and IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Exemplary molecular targets for antibodies encompassed by the present invention also include CD proteins such as CD3, CD4, CD8, CD19, CD20, CD22, CD34 and CD40; members of the ErbB receptor family such as the EGF receptor, HER2, HER3 or HER4 receptor; prostate stem cell antigen (PSCA); cell adhesion molecules such as LFA-1, Mac1, p150.95, VLA-4, ICAM-1, VCAM, α4/β7 integrin, and ≢v/β3 integrin including either α or β subunits thereof (e.g. anti-CD11a, anti-CD18 or anti-CD11b antibodies); growth factors such as VEGF; tissue factor (TF); a tumor necrosis factor (TNF) such as TNF-α or TNF-β, alpha interferon (α-IFN); an interleukin, such as IL-8; IgE; blood group antigens; flk2/flt3 receptor; obesity (OB) receptor; mpl receptor; CTLA-4; protein C etc.

The HER2 antigen to be used for production of antibodies may be, e.g., a soluble form of the extracellular domain of a HER2 receptor or a portion thereof, containing the desired epitope. Alternatively, cells expressing HER2 at their cell surface (e.g. NIH-3T3 cells transformed to overexpress HER2; or a carcinoma cell line such as SK-BR-3 cells, see Stancovski et al. PNAS (USA) 88:8691-8695 (1991)) can be used to generate antibodies. Other forms of HER2 receptor useful for generating antibodies will be apparent to those skilled in the art.

Further antibodies included within the scope of the invention are antibodies to proteins of human cytomegalovirus (HCMV), such as anti-Complex I antibodies and anti-gH antibodies.

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

(viii) Exemplary Anti-HER2 (Anti-ErbB2) Antibodies

The HER2 antibody may, for example, be Pertuzumab. In one embodiment of a HER2 antibody composition, the composition comprises a mixture of a main species Pertuzumab antibody and one or more variants thereof. The preferred embodiment herein of a Pertuzumab main species antibody is one comprising the variable light and variable heavy amino acid sequences in SEQ ID Nos. 7 and 8, and most preferably comprising a light chain amino acid sequence of SEQ ID No. 11, and a heavy chain amino acid sequence of SEQ ID No. 12 (including deamidated and/or oxidized variants of those sequences). In one embodiment, the composition comprises a mixture of the main species Pertuzumab antibody and an amino acid sequence variant thereof comprising an amino-terminal leader extension. Preferably, the amino-terminal leader extension is on a light chain of the antibody variant (e.g. on one or two light chains of the antibody variant). The main species HER2 antibody or the antibody variant may be an full length antibody or antibody fragment (e.g. Fab of F(ab′)2 fragments), but preferably both are full length antibodies. The antibody variant herein may comprise an amino-terminal leader extension on any one or more of the heavy or light chains thereof. Preferably, the amino-terminal leader extension is on one or two light chains of the antibody. The amino-terminal leader extension preferably comprises or consists of VHS-. Presence of the amino-terminal leader extension in the composition can be detected by various analytical techniques including, but not limited to, N-terminal sequence analysis, assay for charge heterogeneity (for instance, cation exchange chromatography or capillary zone electrophoresis), mass spectrometry, etc. The amount of the antibody variant in the composition generally ranges from an amount that constitutes the detection limit of any assay (preferably N-terminal sequence analysis) used to detect the variant to an amount less than the amount of the main species antibody. Generally, about 20% or less (e.g. from about 1% to about 15%, for instance from 5% to about 15%) of the antibody molecules in the composition comprise an amino-terminal leader extension. Such percentage amounts are preferably determined using quantitative N-terminal sequence analysis or cation exchange analysis (preferably using a high-resolution, weak cation-exchange column, such as a PROPAC WCX-10™ cation exchange column). Aside from the amino-terminal leader extension variant, further amino acid sequence alterations of the main species antibody and/or variant are contemplated, including but not limited to an antibody comprising a C-terminal lysine residue on one or both heavy chains thereof, a deamidated antibody variant, etc.

Moreover, the main species antibody or variant may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialidated oligosaccharide attached to one or two heavy chains thereof etc.

The composition may be recovered from a genetically engineered cell line, e.g. a Chinese Hamster Ovary (CHO) cell line expressing the HER2 antibody, or may be prepared by peptide synthesis.

For more information regarding exemplary Pertuzumab compositions, see U.S. Pat. Nos. 7,560,11 and 7,879,325 as well as US 2009/0202546A1.

Another HER2 antibody herein is Trastuzumab. The Trastuzumab composition generally comprises a mixture of a main species antibody (comprising light and heavy chain sequences of SEQ ID NOS: 13 and 14, respectively), and variant forms thereof, in particular acidic variants (including deamidated variants). Preferably, the amount of such acidic variants in the composition is less than about 25%, or less than about 20%, or less than about 15%. See, U.S. Pat. No. 6,339,142. See, also, Harris et al., J. Chromatography, B 752:233-245 (2001) concerning forms of Trastuzumab resolvable by cation-exchange chromatography, including Peak A (Asn30 deamidated to Asp in both light chains); Peak B (Asn55 deamidated to isoAsp in one heavy chain); Peak 1 (Asn30 deamidated to Asp in one light chain); Peak 2 (Asn30 deamidated to Asp in one light chain, and Asp102 isomerized to isoAsp in one heavy chain); Peak 3 (main peak form, or main species antibody); Peak 4 (Asp102 isomerized to isoAsp in one heavy chain); and Peak C (Asp102 succinimide (Asu) in one heavy chain). Such variant forms and compositions are included in the invention herein.

(ix) Exemplary Anti-Complex I Antibodies

In certain embodiments, an anti-Complex I antibody specifically binds to a conformational epitope resulting from the association of UL128, UL130, UL131 with gH/gL or to an epitope within an individual member of Complex I. In some embodiments, the anti-Complex I antibodies neutralize HCMV with an EC90 of 0.7 μg/ml, 0.5 μg/ml, 0.3 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015, 0.012 μg/ml, 0.011 μg/ml, 0.010 μg/ml or less. In other aspects the anti-Complex I antibodies specifically bind to Complex I on the surface of HCVM and neutralize 50% of HCMV at an antibody concentration of 0.05 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml, 0.0009 μg/ml, 0.0008 μg/ml, 0.0007 μg/ml or less (e.g., at an antibody concentration of 10⁻⁸ M, 10⁻⁹M 10⁻¹⁰M, 10⁻¹¹M, 10⁻¹²M, 10⁻¹³M, or lower).

In one aspect, the anti-Complex I antibody comprises at least one, two, three, four, five, or six HVRs selected from (a) HVR-H1 comprising the amino acid sequence of SEQ ID NO:54; (b) HVR-H2 comprising the amino acid sequence of SEQ ID NO:55; (c) HVR-H3 comprising the amino acid sequence of SEQ ID NO:56; (d) HVR-H1 comprising the amino acid sequence of SEQ ID NO:57; (e) HVR-L2 comprising an amino acid sequence selected from the group consisting of SEQ ID NOs:58-67; and (f) HVR-L3 comprising the amino acid sequence of SEQ ID NO:68.

In one aspect, the antibody comprises at least one, at least two, or all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56.

In another aspect, the antibody comprises at least one, at least two, or all three V_{L} HVR sequences selected from (a) HVR-L1 comprising the amino acid sequence of SEQ ID NO:57; (b) HVR-L2 comprising an amino acid sequence selected from SEQ ID NOs:58-67; and (c) HVR-L3 comprising the amino acid sequence of SEQ ID NO:68.

In one embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:58; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:59; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:60; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:61; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:62; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:63; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:64; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:65; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:66; and (c) HVR-L3 comprising the amino acid sequence of SEQ ID NO:68.

In another embodiment, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 comprising the amino acid sequence of SEQ ID NO:67; and (c) HVR-L3 comprising the amino acid sequence of SEQ ID NO:68.

In some embodiments, the antibody comprises all three V_{H} HVR sequences selected from (a) HVR-H1 comprising the amino acid sequence of SEQ ID NO:54; (b) HVR-H2 comprising the amino acid sequence of SEQ ID NO:55; and (c) HVR-H3 comprising the amino acid sequence of SEQ ID NO:56 and three V_{L} HVR sequences selected from (a) HVR-L1 comprising the amino acid sequence of SEQ ID NO:57; (b) HVR-L2 and the first amino acid of the light chain variable region framework FR3 comprising the amino acid sequence of SEQ ID NO:69; and (c) HVR-L3 comprising the amino acid sequence of SEQ ID NO:68. In certain embodiments, any one or more amino acids of an anti-Complex I antibody as provided above are substituted at the following HVR positions: in HVR-L2 (SEQ ID NO:58): positions 4, 5, 11, and 12. In certain embodiments, the substitutions are conservative substitutions, as provided herein. In certain embodiments, any one or more of the following substitutions may be made in any combination: in HVR-L2 (SEQ ID NO:24): D4E, D4T, D4S, G5A, D11E, D11T, D11S, and G12A. All possible combinations of the above substitutions are encompassed by the consensus sequences of SEQ ID NO:69.

In any of the above embodiments, an anti-Complex I antibody is humanized. In one embodiment, an anti-Complex I antibody comprises HVRs as in any of the above embodiments, and further comprises an acceptor human framework, e.g. a human immunoglobulin framework or a human consensus framework. In another embodiment, an anti-Complex I antibody comprises HVRs as in any of the above embodiments, and further comprises a V_{H} comprising an FR1 sequence of SEQ ID NO:70, an FR2 sequence of SEQ ID NO:71, an FR3 sequence of SEQ ID NO:72, and an FR4 sequence of SEQ ID NO:73. In other embodiments, the anti-Complex I antibody comprises HVRs as in any of the above embodiments, and further comprises a V_{H} comprising an FR1 sequence of SEQ ID NO:70, a FR2 sequence of SEQ ID NO:74, a FR3 sequence of SEQ ID NO:75, and a FR4 sequence of SEQ ID NO:76. In other embodiments the anti-Complex I antibody comprises HVRs as in any of the above embodiments, and further comprises a V_{H} comprising an FR1 sequence of SEQ ID NO:77, a FR2 sequence of SEQ ID NO:78, a FR3 sequence of SEQ ID NO:79, and a FR4 sequence of SEQ ID NO:73. In other embodiments the anti-Complex I antibody comprises HVRs as in any of the above embodiments, and further comprises a V_{H} comprising an FR1 sequence of SEQ ID NO:80, a FR2 sequence of SEQ ID NO:71, a FR3 sequence of SEQ ID NO:81, and a FR4 sequence of SEQ ID NO:73.

In another embodiment, an anti-Complex I antibody comprises HVRs as in any of the above embodiments, and further comprises a V_{L} comprising an FR1 sequence of SEQ ID NO:82, an FR2 sequence of SEQ ID NO:83, an FR3 sequence of SEQ ID NO:84, and an FR4 sequence of SEQ ID NO:85. In other embodiments the anti-Complex I antibody comprises HVRs as in any of the above embodiments, and further comprises a V_{L} comprising an FR1 sequence of SEQ ID NO:86, a FR2 sequence of SEQ ID NO:87, a FR3 sequence of SEQ ID NO:88, and a FR4 sequence of SEQ ID NO:89. In other embodiments the anti-Complex I antibody comprises HVRs as in any of the above embodiments, and further comprises a V_{L} comprising an FR1 sequence of SEQ ID NO:90, a FR2 sequence of SEQ ID NO:91, a FR3 sequence of SEQ ID NO:88, and a FR4 sequence of SEQ ID NO:89.

In any of the above antibodies, the V_{L}FR3 sequence may be substituted with one selected from SEQ ID NO:92 or SEQ ID NO:93.

In another aspect, an anti-Complex I antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:37 or SEQ ID NO:95. In certain embodiments, a V_{H} sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Complex I antibody comprising that sequence retains the ability to bind to Complex I. In certain embodiments, a total of 1 to 10 amino acids have been substituted, inserted and/or deleted in SEQ ID NO:35, or SEQ ID NO:37, or SEQ ID NO:95. In certain embodiments, substitutions, insertions, or deletions occur in regions outside the HVRs (i.e., in the FRs). In a particular embodiment, the V_{H} comprises one, two or three HVRs selected from: (a) HVR-H1 comprising the amino acid sequence of SEQ ID NO:54, (b) HVR-H2 comprising the amino acid sequence of SEQ ID NO:55, and (c) HVR-H3 comprising the amino acid sequence of SEQ ID NO:56.

In another aspect, an anti-Complex I antibody is provided, wherein the antibody comprises a light chain variable domain (VL) having at least 90%, 91%, 92%, 93%, 94%, 95%, 96%, 97%, %, 99%, or 100% sequence identity to the amino acid sequence of SEQ ID NO:34 or SEQ ID NO:38. In certain embodiments, a V_{L} sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Complex I antibody comprising that sequence retains the ability to bind to Complex I. In certain embodiments, a total of 1 to 10 amino acids have been substituted, inserted and/or deleted in SEQ ID NO:34 or SEQ ID NO:38. In certain embodiments, the substitutions, insertions, or deletions occur in regions outside the HVRs (i.e., in the FRs). In a particular embodiment, the V_{L} comprises one, two or three HVRs selected from (a) HVR-L1 comprising the amino acid sequence of SEQ ID NO:57; (b) HVR-L2 comprising the amino acid sequence selected from SEQ ID NOs:58-67; and (c) HVR-L3 comprising the amino acid sequence of SEQ ID NO:68.

In another aspect, an anti-Complex I antibody is provided, wherein the antibody comprises a V_{H} as in any of the embodiments provided above, and a V_{L} as in any of the embodiments provided above. In one embodiment, the antibody comprises the V_{H} and V_{L} sequences in SEQ ID NO:35 and SEQ ID NO:38, respectively, including post-translational modifications of those sequences. In one embodiment, the antibody comprises the V_{H} and V_{L} sequences in SEQ ID NO:37 and SEQ ID NO:38, respectively, including post-translational modifications of those sequences. In another embodiment, the antibody comprises the V_{H} and V_{L} sequences in SEQ ID NO:95 and SEQ ID NO:38, respectively, including post-translational modifications of those sequences. In another embodiment, the antibody comprises the V_{H} and V_{L} sequences in SEQ ID NO:35 and SEQ ID NO:34, respectively, including post-translational modifications of those sequences. In another embodiment, the antibody comprises the V_{H} and V_{L} sequences in SEQ ID NO:37 and SEQ ID NO:34, respectively, including post-translational modifications of those sequences. In another embodiment, the antibody comprises the V_{H} and V_{L} sequences in SEQ ID NO:95 and SEQ ID NO:34, respectively, including post-translational modifications of those sequences.

In a further aspect, the invention provides an antibody that competes with and/or binds to the same epitope as an anti-Complex I antibody provided herein. For example, in certain embodiments, an antibody is provided that competes with and/or binds to the same epitope as an anti-Complex I antibody comprising a V_{H} comprising an amino acid sequences of SEQ ID NOs:35, 37, or 95 and a V_{L} comprising an amino acid sequence of SEQ ID NO:34 or SEQ ID NO:38.

In a further aspect, the invention provides an antibody that binds to the same epitope as an anti-Complex I antibody comprising amino acids which correspond to the amino acids selected from glutamine at amino acid position 47 of SEQ ID NO:97, lysine at amino acid position 51 of SEQ ID NO:97; aspartic acid at amino acid position 46 of SEQ ID NO:97 and combinations thereof. The corresponding amino acids which comprise the epitope may be at approximately the same location in the UL131 amino acid sequence but may differ due to amino acid sequence differences in UL131 between various HCMV strains.

In a further aspect, the invention provides an antibody that binds to a polypeptide of HCMV Complex I, wherein the polypeptide comprises the amino acid sequence SRALPDQTRYK YVEQLVDLTLNYHYDAS (SEQ ID NO:98).

In a further aspect, the antibody binds to the same epitope as an anti-Complex I antibody disclosed herein. In additional aspects, the antibody binds to the same epitope as an anti-Complex I antibody disclosed herein with an EC90 of 0.7 0.5 μg/ml, 0.3 μg/ml, 0.1 μg/ml, 0.09 μg/ml, 0.08 μg/ml, 0.07 μg/ml, 0.06 μg/ml, 0.05 μg/ml, 0.04 μg/nil, 0.03 μg/ml, 0.02 μg/ml, 0.015, 0.012 μg/ml, 0.011 μg/ml, 0.010 μg/ml or less. In other aspects the antibody binds to the same epitope as an anti-Complex I antibody disclosed herein and which neutralizes 50% of HCMV at an antibody concentration of 0.05 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/m.1, 0.007 μg/ml, 0.006 μg/ml, 0.005 μg/ml, 0.004 μg/ml, 0.003 μg/ml, 0.002 μg/ml, 0.001 μg/ml, 0.0009 μg/ml, 0.0008 μg/ml, 0.0007 μg/ml or less (e.g., at an antibody concentration of 10 ⁻⁸ M, 10⁻⁹ M 10^{'10} M, 10⁻¹¹ M, 10⁻¹² M, M 10⁻¹³ M, or lower).

In a further aspect of the invention, an anti-Complex I antibody according to any of the above embodiments is a monoclonal antibody, including a chimeric, humanized or human antibody. In one embodiment, an anti-Complex I antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

III. Pharmaceutical Formulations

Therapeutic formulations of the antibodies or antibody-like molecules used in accordance with the present invention are prepared for storage by mixing an antibody or antibody-like molecule having the desired degree of purity with optional pharmaceutically acceptable carriers, excipients or stabilizers (Remington's Pharmaceutical Sciences 16th edition, Osol, A. Ed. (1980)), generally in the form of lyophilized formulations or aqueous solutions. Antibody crystals are also contemplated (see US Pat Appln 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801, expressly incorporated herein by reference.

Lyophilized antibody formulations are described in U.S. Pat. Nos. 6,267,958, 6,685,940 and 6,821,515, expressly incorporated herein by reference. The preferred HERCEPTIN® (Trastuzumab) formulation is a sterile, white to pale yellow preservative-free lyophilized powder for intravenous (IV) administration, comprising 440 mg Trastuzumab, 400 mg .alphaα,α-trehalose dehydrate, 9.9 mg L-histidine-HCl, 6.4 mg L-histidine, and 1.8 mg polysorbate 20, USP. Reconsitution of 20 mL of bacteriostatic water for injection (BWFI), containing 1.1% benzyl alcohol as a preservative, yields a multi-dose solution containing 21 mg/mL Trastuzumab, at pH of approximately 6.0. For further details, see the Trastuzumab prescribing information.

The preferred Pertuzumab formulation for therapeutic use comprises 30 mg/mL Pertuzumab in 20 mM histidine acetate, 120 mM sucrose, 0.02% polysorbate 20, at pH 6.0. An alternate Pertuzumab formulation comprises 25 mg/mL Pertuzumab, 10 mM histidine-HCl buffer, 240 mM sucrose, 0.02% polysorbate 20, pH 6.0.

The anti-Complex I antibodies and anti-gH antibodies can be formulated in a similar fashion. The two antibodies are manufactured separately, and, in one embodiment, are formulated at 20 mg/ml in 20 mM histidine acetate, 240 mM sucrose, 0.02% polysorbate 20, pH 5.5. The formulations upon dilution in a pharmaceutically acceptable diluent support intravenous delivery.

The formulation of a placebo is equivalent to the antibody formulation, without the active agent.

The formulations herein may also contain more than one active compound as necessary for the particular indication being treated, preferably those with complementary activities that do not adversely affect each other.

The formulations to be used for in vivo administration must be sterile. This is readily accomplished by filtration through sterile filtration membranes.

IV. Articles of Manufacture

One embodiment of an article of manufacture herein comprises an intravenous (IV) bag containing a stable mixture of at least two antibodies and/or antibody-like molecules, formulated separately, suitable for administration to a patient in need. Optionally, the mixture is in saline solution; for example comprising about 0.9% NaCl or about 0.45% NaCl. An exemplary IV bag is a polyolefin or polyvinyl chloride infusion bag, e.g. a 250 mL IV bag.

Optionally, the mixture in the IV bag is stable for up to about 8 hours, or up to about 12 hours, or up to about 24 hours at 5° C. or 30° C. Optionally, the mixture in the IV bag is stable for at least about 8 hours, or at least about 12 hours, or at least about 24 hours at 5° C. or 30° C. Stability of the mixture can be evaluated by one or more assays selected from the group consisting of: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay.

According to one embodiment of the invention, the mixture includes about 420 mg or about 840 mg of Pertuzumab and from about 200 mg to about 1000 mg of Trastuzumab (e.g. from about 400 mg to about 900 mg of Trastuzumab). In another embodiment, the mixture includes an anti-Complex I antibody and an anti-gH antibody. In one embodiment, the anti-Complex I antibody is used in a concentration of 1.74 Au/1 mg and the anti-gH antibody is used in a concentration of 1.47 Au/1 mg/ In this case, the total AU for the combination (2 mg/ml) will be 3.21 AU. In another embodiment, the ratios of the anti-Complex I antibody and the anti-gH antibody with a final combined concentration of about 0.5 mg/mL is between about 0.77-0.89 AU.

In a further embodiment, the mixture includes an anti-HER2 antibody and an immunoconjugate, such as T-DM1.

VII. Deposit of Biological Materials

The following hybridoma cell lines have been deposited with the American Type Culture Collection, 10801 University Boulevard, Manassas, Va. 20110-2209, USA (ATCC):

| Antibody Designation | ATCC No. | Deposit Date |
|---|---|---|
| 4D5 | ATCC CRL 10463 | May 24, 1990 |
| 2C4 | ATCC HB-12697 | Apr. 8, 1999 |

Further details of the invention are illustrated by the following non-limiting Examples. The disclosures of all citations in the specification are expressly incorporated herein by reference.

### EXAMPLE 1

### Co-Administration of Pertuzumab and Trastuzumab From a Single Infusion Bag

In the phase III clinical trials above Pertuzumab was administered by intravenous (IV) infusion in saline IV bags to patients with HER2-positive metastatic breast cancer followed by Trastuzumab and the chemotherapeutic agent Docetaxel also using saline IV infusions. The IV infusion process for Pertuzumab and Trastuzumab takes approximately 60 to 90 minutes each with a 30 to 60 minute patient observation period after each drug. Due to this treatment regimen per patient, a visit can take up to 7.5 hours total. As medical payments for both drugs and drug administration services have been under scrutiny in the recent past, there has been emphasis on business practices to shorten time and to increase medical resource utilization in clinical and hospital settings. Increased efficiency of patient care, compliance and treatment is expected by shortening the time patients spend in the clinic for each cycle of treatment.

As part of the phase III Pertuzumab clinical trials, Pertuzumab and Trastuzumab are administered through intravenous (IV) infusion to patients sequentially, i.e. one drug after the other. While Pertuzumab is given as a flat dose (420 mg for maintenance, 840 mg for loading), Trastuzumab is weight based (6 mg/kg for maintenance doses). To increase convenience and minimize the in-clinic time for the patients, the feasibility of co-administering Pertuzumab with Trastuzumab in a single 250 mL 0.9% saline polyolefin (PO) or polyvinyl chloride (PVC) IV infusion bag was assessed. The individual monoclonal antibodies have been demonstrated to be stable in infusion bags (PO and/or PVC) over 24 hours at 5° C. and 30° C. In this study, the compatibility and stability of Pertuzumab (420 mg and 840 mg) mixed with either 420 mg Trastuzumab (6 mg/kg dose for a 70 kg patient) or 720 mg (6 mg/kg for a 120 kg patient) in IV bags for up to 24 hours at 5° C. or 30° C. was evaluated. The controls (i.e. Pertuzumab alone in an IV bag, Trastuzumab alone in an IV bag) and the monoclonal antibody (mAb) mixture samples were assessed using the existing Pertuzumab and Trastuzumab analytical methods, which include color, appearance and clarity (CAC), concentration and turbidity by UV-spec scan, particulate analysis by HIAC-Royco, size exclusion chromatography (SEC), and ion-exchange chromatography (IEC). Additionally, capillary zone electrophoresis (CZE), image capillary isoelectric focusing (iCIEF), and potency (the Pertuzumab anti-proliferation assay only) was utilized to measure the admixtures containing 1:1 of Pertuzumab:Trastuzumab and their respective controls (420 mg Pertuzumab only and 420 mg Trastuzumab only) only as a representative case.

Results showed no observable differences by the above assays in the Pertuzumab/Trastuzumab mixtures between the time zero (T0) control and the sample stored up to 24 hours at either 5° C. or 30° C. The physicochemical assays as listed above were able to detect both molecules as well as the minor variants in the drug mixture, though some overlaps of monoclonal antibody species were seen in the chromatograms. Furthermore, the drug mixture tested by the Pertuzumab specific inhibition of cell proliferation assay showed comparable potency before and after storage. The results from this study showed the Pertuzumab and Trastuzumab admixtures are physically and chemically stable in an IV infusion bag for up to 24 hours at 5° C. or 30° C. and can be used for clinical administration if necessary.

Dose I: 840 mg Pertuzumab/Trastuzumab Mixture (420 mg Pertuzumab and 420 mg Trastuzumab)

Sample Preparation: All procedures were performed aseptically under a laminar flow hood. PO IV infusion bag samples with three types of drug combinations were prepared for this study: 1) 420 mg Pertuzumab/420 mg Trastuzumab mixture, 2) 420 mg Pertuzumab alone, and 3) 420 mg Trastuzumab alone. The Pertuzumab and Trastuzumab alone samples served as controls.

Trastuzumab was reconstituted with 20 mL of bacteriostatic water for injection (BWFI) and left on lab bench for approximately 15 minutes prior to use. To prepare the Pertuzumab/Trastuzumab sample dose, 14 mL of Pertuzumab (420 mg) was diluted directly into the IV infusion bag that contained a nominal 250 mL 25 mL overage) 0.9% saline solution, without removing an equal amount of saline, followed by 20 mL of the reconstituted Trastuzumab (420 mg) using an 18 gauge needle at room temperature. The total concentration of the two proteins combined in the 250 mL IV bag was expected to be approximately 3 mg/mL. Similarly, the Pertuzumab (420 mg) alone IV bag was prepared with 14 mL of the 30 mg/mL drug product directly diluted into an IV infusion bag. The final expected concentration was approximately 1 mg/mL. The Trastuzumab (420 mg) alone IV infusion bag was also prepared in the same manner except 20 mL of the 21 mg/mL drug product was added into the bag. The final expected concentration was approximately 1 mg/mL.

The PO IV bags were manually mixed thoroughly by a gentle back and forth rocking motion several times to ensure homogeneity. After mixing, 10 mL of sample was removed with a syringe from each bag and stored in sterile 15 cc falcon tubes to be used as the diluted sample control at time zero (T0). The IV bags were then stored covered in foil at 30° C. for 24 hours (T24). Immediately after storage, the remainder of the sample was removed with a syringe from each bag and placed into sterile 250 mL PETG containers. The T0 and T24 samples were held for up to 24 hours at 5° C. or immediately analyzed by CAC, UV-spec scan (concentration and turbidity), SEC, IEC, CZE, iCIEF, HIAC-Royco, as well as potency. The product quality of the samples was tested by the Pertuzumab and Trastuzumab product specific SEC and IEC methods, while only the Pertuzumab specific potency method was performed. The other assays utilized were non-product specific methods. All assays were qualified for the intended testing in their respective molecules and used without further method optimization.

Dose II: 1560 mg Pertuzumab/Trastuzumab Mixture (840 mg Pertuzumab and 720 mg Trastuzumab)

Sample preparation: The upper range of the mAb co-administration dose was examined (1560 mg total mixture: 840 mg Pertuzumab and 720 mg Trastuzumab) in PO and PVC IV infusion bag samples. In the event that an increase in protein aggregation is observed, the propensity of the formation of high molecular weight species (HMWS) would more likely occur at the upper dose of 1560 mg total mAb rather than the mixture containing 840 mg. To mitigate the risk during in-use conditions at the high dose range, both PO and PVC IV infusion bags were studied to ensure no interactions were seen.

Three types of drug combinations (mixture, 840 mg Pertuzumab alone, and 720 mg Trastuzumab alone) were prepared and handled similar to the dose I study. The Pertuzumab/Trastuzumab mixture contained 28 mL of Pertuzumab (840 mg) diluted directly into either PO or PVC IV infusion bags followed by 34 mL of the reconstituted Trastuzumab (720 mg) using an 18 gauge needle at room temperature. The total concentration of the two mAbs combined in a single 250 mL IV bag was expected to be approximately 5 mg/mL. For the controls, Pertuzumab and Trastuzumab alone IV infusion bag samples were prepared and handled similar to the dose I study, except 28 mL of 30 mg/mL Pertuzumab and 34 mL of 21 mg/mL Trastuzumab was directly diluted into each PO or PVC IV infusion bag. The final expected concentration was approximately 3 mg/mL for the Pertuzumab (840 mg) and Trastuzumab (720 mg) alone samples. The bags were stored uncovered at either 5° C. or 30° C. for up to 24 hours. The T0 and T24 samples were analyzed immediately or held for up to 24 to 48 hours at 5° C. by CAC, UV-spec scan (concentration and turbidity), SEC, IEC, and HIAC-Royco.

Details of the types of doses, IV infusion bags, dose & preparation, storage temperatures, and assays are summarized in Table 1.

TABLE 1
IV Bag Type, Dose, Preparation & Study Conditions

| | | | | |
|---|---|---|---|---|
| IV bag type | | Dilution | Storage | |
| (approx 250 mL, | Total dose, | (into approx | temperature | |
| 0.9% NaCl) | concentration | 250 mL IV bag) | (up to 24 hrs) | Assays |

Dose I (n = 1)

| | | | | |
|---|---|---|---|---|
| PO | 840 mg, | Add 14 mL P (~30 mg/mL) + | 30° C. | CAC, UV spec scan |
| | approx 3 mg/mL | 20 mL T (~21 mg/mL) | | (concentration, |
| | 420 mg^{b}, | Add 14 mL P (~30 mg/mL) | | turbidity), SEC, IEC, |
| | approx 2 mg/mL | | | CZE, icIEF, HIAC- |
| | 420 mg^{b}, | Add 20 mL T (~21 mg/mL) | | Royco, potency |
| | approx 1 mg/mL | | | |

Dose II (n = 1)

| | | | | |
|---|---|---|---|---|
| PO and PVC^{a} | 1560 mg, | Add 28 mL P (~30 mg/mL) + | 5° C. | CAC, UV spec scan |
| | approx 5 mg/mL | 34 mL T (~21 mg/mL) | 30° C. | (concentration & |
| PO and PVC | 840 mg^{b}, | Add 28 mL P (~30 mg/mL) | | turbidity), SEC, IEC, |
| | approx 3 mg/mL | | | HIAC-Royco |
| | 720 mg^{b}, | Add 34 mL T (~21 mg/mL) | | |
| | approx 3 mg/mL | | | |

^{a}n = 2
^{b}control
P = Pertuzumab;
T = Trastuzumab

### Assays

All samples were held at 5° C. or immediately analyzed. Typically, samples were analyzed within 24 to 48 hours of preparation and storage. The following assays were conducted to ascertain product quality and short term stability of Pertuzumab/ Trastuzumab mixture, Pertuzumab alone, and Trastuzumab alone samples diluted into saline IV infusion bags. Since several assays, i.e. SEC, IEC, CZE, iCIEF, and potency, were not optimized for quantitative assessment of the mAb mixtures, only chromatographic or electropherographic overlays of these samples and their individual controls before and after storage at 5° C. or 30° C. are shown here. For consistency, no values, e.g. percent peak area, were calculated for all three sample types from the liquid chromatography and electrophorectic assays that were performed.

### Color, Appearance, and Clarity (CAC)

The color, appearance, and clarity of the samples were determined by visual inspection under a white fluorescence light with black and white background at room temperature. A 3 cc glass vial was filled with 1 mL of each sample for CAC testing. A negative control (purified water) with the corresponding sample volume was used for comparison.

### UV-Vis Spectrophotometer Scan for Concentration Measurements

The concentration was determined by measurement of the UV-absorbance on an HP8453 spectrophotometer via volumetric sample preparation. The instrument was blanked with 0.9% saline. Absorbance at Aₘₐₓ(278 nm or 279 nm) and 320 nm in a quartz cuvette with 1-cm path length were measured for each sample. The absorbance at 320 nm is used to correct for background light scattering in solution. The concentration determination was calculated by using the absorptivity of 1.50 (mg/mL)⁻¹cm⁻¹ for both Pertuzumab and Trastuzumab molecules.

$$Protein Concentration ( mg \text{/} mL ) = \frac{{A}_{max} - {A}_{320}}{1.50} \times Dilution Factor \times \frac{1}{cuvette pathlength}$$

### Size Exclusion Chromatography (SEC: Pertuzumab Specific and Trastuzumab Specific)

Each sample was injected into a TOSOHAAS® column G3000 SWXL, 7.8×300 mm at ambient temperature on an AGILENT 1100® HPLC. The eluted peaks were monitored at 280 nm. Chromatographic integrations were analyzed by the CHROMELEON® software. The autosampler temperature was held at 2-8° C. throughout the run and mobile phases used were 0.2M potassium phosphate, 0.25 mM potassium chloride, pH 6.2 and 100 mM potassium phosphate, pH 6.8 for Pertuzumab-assay and Trastuzumab-assay, respectively. The recommended injection load as specified by the test procedure was 200 μg with an injection volume of 20 μL. The diluted 420 mg sample was injected at a load less than the recommended amount due to the low concentration of the protein after dilution in the IV bags. The maximum injection volume of the HPLC sample loop was 100 μL, which limits the volume that is able to be injected at one time. As a result, the injection volumes were modified to 100 μL at 160 μg protein for the Pertuzumab alone and Trastuzumab alone samples (420 mg dose group) and 73 μL at 200 μg protein for the Pertuzumab/Trastuzumab mixture (840 mg dose group). Modification in the injection volumes have been utilized in previous IV bag studies and are necessary when handling low concentration samples.

### Ion-Exchange Chromatography (IEC)

The analysis of carboxypeptidase B (CpB)-digested Pertuzumab and Trastuzumab for charge heterogeneity was employed by IEC for each sample. For the Pertuzumab specific IEC, samples were either tested with regular IEC (“Pertuzumab-regular IEC”) or a modified “fast” version of IEC (“Pertuzumab-IEC-fast”) for high throughput, method for the purpose of these experiments. The IEC assays utilized the a DIONEX® WCX weak cation exchange column equilibrated with solvent A (20 mM MES, 1 mM Na₂EDTA pH 6.00) and solvent B (250 mM sodium chloride in solvent A) monitored at 280 nm for Pertuzumab-regular IEC and Pertuzumab-IEC-fast, whereas solvent A (10 mM sodium phosphate, pH 7.5) and solvent B (100 mM sodium chloride in Solvent A) monitored at 214 nm was used for Trastuzumab on an AGILENT 1100® HPLC. The peaks were eluted at a flow rate of 0.8 mL/min with an increasing gradient of 18%-100% solvent B over 35 minutes and 90 minutes for Pertuzumab-regular-IEC and Pertuzumab-IEC-fast, respectively, and 15%-100% solvent B over 55 minutes for Trastuzumab-IEC. Column temperatures were maintained at either 34° C. or 42° C. and ambient for Pertuzumab-regular-IEC or Pertuzumab-fast-IEC and Trastuzumab-IEC, respectively, while the auto sampler temperature was held at 2-8° C. throughout the run.

### HIAC-ROYCO™ Light Obscuration for Sub-Visible Particles

Particulate counts in the diluted drug product were carried out using the HIAC-ROYCO™ Liquid Particulate Counting System model 9703. Average cumulative numbers of particles at ≧10 μm and ≧25 μm per milliliter were tabulated in each sample using PHARMSPEC v2.0 ™. The test procedure was modified for a small- volume method, utilizing either four 1 mL readings or four 0.4 mL readings per a test session while discarding the first reading of each sample. The HIAC-ROYCO™ samples were degassed under vacuum for approximately 10-15 minutes each. The size below 10 μm was not collected for this sample set.

### UV-Vis Spectrophotometer Scan for Turbidity Measurements

The optical density of the samples from the IV bag (1 mg/mL or 3 mg/mL) was measured in a quartz cuvette with a 1-cm path length on a HP8453 spectrophotometer. The sample readings were blanked against purified water. The absorbance measurements were recorded at 340 nm, 345 nm, 350 nm, 355 nm, and 360 nm and the turbidity was expressed as an average of these wavelengths.

### Capillary Zone Electrophoresis, CZE

CZE was performed using a PROTEOMELAB PA800™ capillary electrophoresis system (Beckman Coulter) with neutral-coated capillary (50 μm×50 cm). The buffer consisted of 40 mM ε-amino caproic acid/acetic acid, pH 4.5, 0.2% hydroxypropyl methyl cellulose (HPMC). Samples were diluted to 0.5 mg/mL in water and injected into the capillary at 1 psi for 10 seconds. Separation was performed using a voltage of 30 kV for 15 minutes, and the species were detected by UV at 214 nm.

### CE-SDS-LIF, Reduced and Non-Reduced

Each sample was derivatized with 5 carboxytetramethylrhodamine succinimidyl ester, a fluorescent dye. After removing the free dye through gel filtration (using NAP-5 columns), non-reduced samples were prepared by adding 40 mM iodoacetamide and heated at 70° C. for 5 minutes. For the analysis of the reduced samples, the derivatized samples were mixed with SDS to a final concentration of 1% (v/v) and 10 mL of a solution containing 1 M DTT, and heated at 70° C. for 20 minutes. The prepared samples were analyzed on a Beckman Coulter ProteomeLab PA800 system using a 50 mm I.D. 31.2 cm fused silica capillary maintained at 20° C. throughout the analysis. Samples were introduced into the capillary by electrokinetic injection at 10 kV for 40 seconds. The separation was conducted at a constant voltage of 15 kV in the reversed polarity (negative to positive) mode using CE-SDS running buffer as the sieving medium. An argon ion laser operating at 488 nm was used for fluorescence excitation with the resulting emission signal monitored at 560 nm.

iCIEF

The distribution of charge variants of the Pertuzumab/Trastuzumab mixture, Pertuzumab alone, and Trastuzumab alone was assessed by iCIEF using an iCE280™ analyzer (Convergent Bioscience) with a fluorocarbon coated capillary cartridge (100 μm×5 cm). The ampholyte solution consisted of a mixture of 0.35% methyl cellulose (MC), 0.47% Pharmalyte 3-10 carrier ampholytes, 2.66% Pharmalyte 8-10.5 carrier ampholytes, and 0.20% pI markers 7.05 and 9.77 in purified water. The anolyte was 80 mM phosphoric acid, and the catholyte was 100 mM sodium hydroxide, both in 0.10% methylcellulose. Samples were diluted in purified water and CpB was added to each diluted sample at an enzyme to substrate ratio of 1:100 followed by incubation at 37° C. for 20 minutes. The CpB treated samples were mixed with the ampholyte solution and then focused by introducing a potential of 1500 V for one minute, followed by a potential of 3000 V for 10 minutes. An image of the focused Pertuzumab charge variants was obtained by passing 280 nm ultraviolet light through the capillary and into the lens of a charge coupled device digital camera. This image was then analyzed to determine the distribution of the various charge variants.

### Anti-Proliferation Potency Assay

This test procedure is based on the ability of Pertuzumab to inhibit the proliferation of MDA MB 175 VII human breast carcinoma cells. Briefly, cells were seeded in 96-well tissue culture microtiter plates and incubated overnight at 37° C. under 5% CO₂ to allow cell attachment. The following day, the culture medium was removed and serial dilutions of each standard, controls, and sample(s) were added to the plates. The plates were then incubated for four days at 37° C. under 5% CO₂ and the relative number of viable cells was quantified indirectly using a redox dye, ALAMARBLUE® according to the manufacturer's protocol. Each sample was assayed in triplicate and the changes in color as measured by fluorescence were directly proportional to the number living cells in the culture. The absorbance of each well was then measured on a fluorescence 96-well plate reader. The results, expressed in relative fluorescence units (RFU), were plotted against the antibody concentration. No quantitative measurements were made, or possible, since there was no Pertuzumab/Trastuzumab mixture reference available. Therefore, the results are comparisons of the dose response curves only.

### Results and Discussion

Dose I: 840 mg total Pertuzumab/Trastuzumab mixture (420 mg Pertuzumab and 420 mg Trastuzumab)

The product quality of the total 840 mg Pertuzumab/Trastuzumab mixture (420 mg Pertuzumab and 420 mg Trastuzumab), Pertuzumab alone (420 mg), and Trastuzumab alone (420 mg) in IV infusion bags (n=1) before and after storage at 30° C. for up to 24 hours was assessed by CAC, concentration measurements by UV-spec scan, turbidity, and HIAC Royco (Table 2). The Pertuzumab and Trastuzumab alone IV infusion bags are considered controls that were also prepared to assess the ability of the assay to pick up the appropriate product attributes.

TABLE 2
Dose I 840 mg: Stability data for Pertuzumab/Trastuzumab mixture, Pertuzumab, or Trastuzumab
in 0.9% saline PO IV infusion bags (n = 1)

| | | | | | | | | Light Obscuration | |
|---|---|---|---|---|---|---|---|---|---|
| | IV bag | Amount | Timepoint | Temp | CAC^{a} | Conc. | Turbidity | total particles | total particles |
| Sample | type | mg | Hour(s) | ° C. | liquid | mg/mL | AU | ≧10 um/mL | ≧25 um/mL |
| pertuzumab/ | PO | 840 | 0 | 30 | CL, CO | 2.7 | 0.016 | 1 | 0 |
| trastuzumab | | 840 | 24 | 30 | CL, CO | 2.7 | 0.016 | 6 | 0 |
| mixture | | | | | | | | | |
| pertuzumab | PO | 420 | 0 | 30 | CL, CO | 1.4 | 0.012 | 3 | 0 |
| | | 420 | 24 | 30 | CL, CO | 1.4 | 0.011 | 4 | 0 |
| trastuzumab | PO | 420 | 0 | 30 | CL, CO | 1.5 | 0.012 | 1 | 0 |
| | | 420 | 24 | 30 | CL, CO | 1.5 | 0.011 | 6 | 0 |
| saline only | | — | — | — | — | — | — | 2 | 1 |

RT = room temperature
^{a}Color, appearance, and clarity: CL = clear; SOPL = slightly opalescent, CO = colorless.

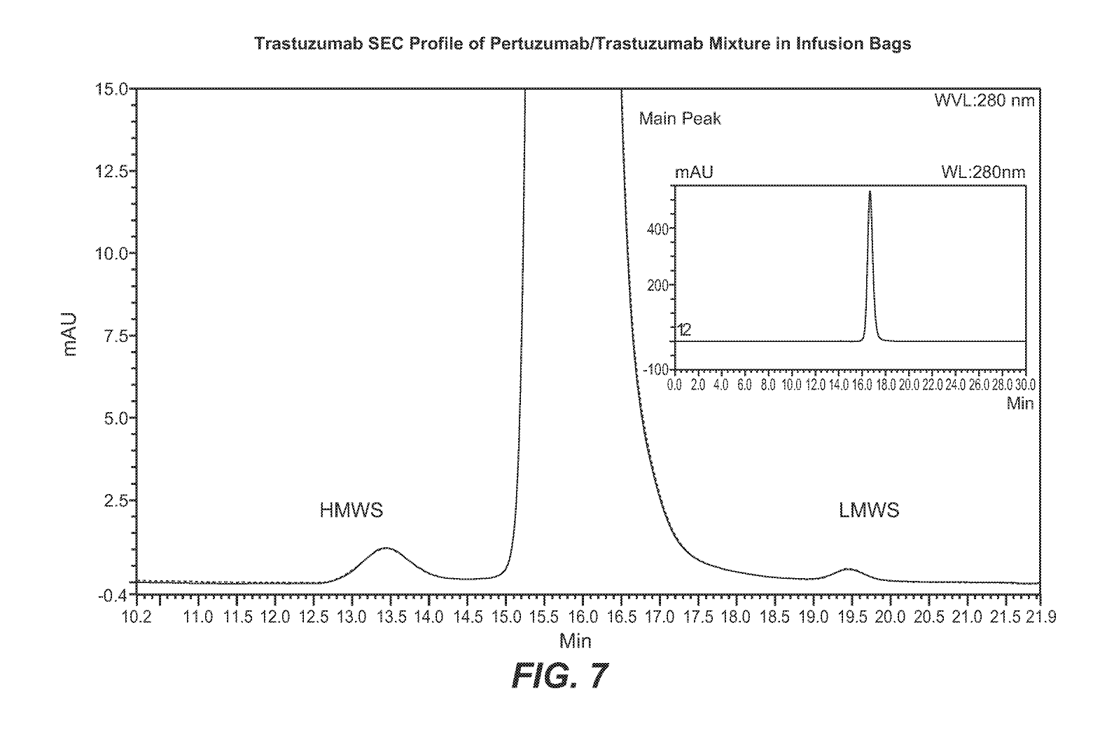

After storage, the Pertuzumab/Trastuzumab mixture, Pertuzumab alone, and Trastuzumab alone samples appeared as a clear and colorless liquid with no visible particles as observed by CAC. The concentration and turbidity measurements showed no measureable changes in any of the three sample types after 24 hours at 30° C. Particulate analysis by HIAC Royco detected no more than 6 particles greater than or equal to 10 μm size and no particles greater than 25 μm size for Pertuzumab/Trastuzumab mixture, Pertuzumab alone, or Trastuzumab alone samples post storage. These results are comparable to the 0.9% saline only solution. The lack of visible precipitation or particulates indicates that the admixture and the controls are sufficiently stable upon dilution in the 0.9% saline IV infusion bags. The Pertuzumab/Trastuzumab mixture diluted in saline were run on SEC, both Pertuzumab and Trastuzumab specific methods, and showed comparable peak profiles between T0 and T24 (FIGS. 6 and 7). No increases were observed in the high molecular weight species (HMWS) and low molecular weight species (LMWS). Similarly, no changes were observed in the main peak in any sample. The main peak and the peak area of the HMWS and LMWS overlay and cannot be distinguished in the Pertuzumab/Trastuzumab mixture due the size similarity between Pertuzumab and Trastuzumab (molecular weight approximately 150 kD). Furthermore, comparison of T0 and T24 for both the Pertuzumab and Trastuzumab alone sample showed no observable changes in peak area or profile as detected by the two SEC methods listed above.

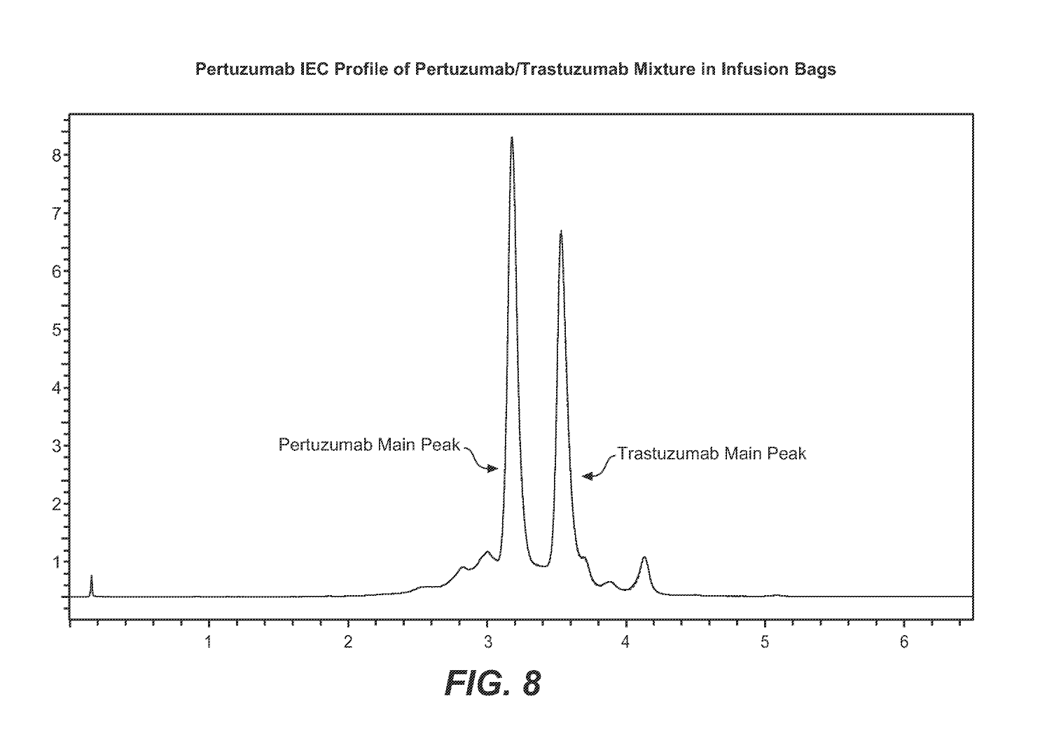

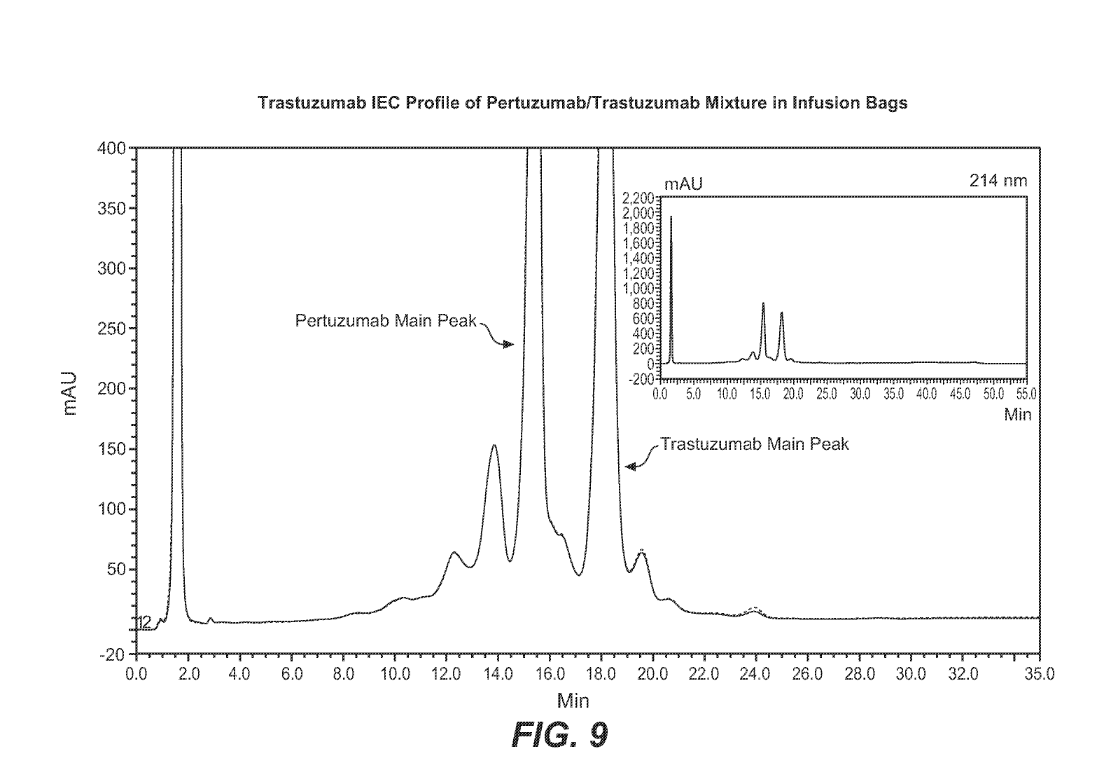

Two product specific methods for Pertuzumab or Trastuzumab IEC was utilized to analyze the Pertuzumab/Trastuzumab mixture (FIGS. 8 and 9). In the cation-exchange chromatography assays, each molecule typically contain three distinct areas that are eluted based on relative charge, with the early eluting acidic variants, followed by the main peak, and lastly the late eluting basic variants. In the Pertuzumab and Trastuzumab alone chromatograms, the profile exhibiting the acidic variants, main peak, and the basic variants was observed and deemed comparable between the starting material and post storage at 30° C. These results are also consistent with prior studies conducted in saline IV infusion bags for the either Pertuzumab or Trastuzumab alone. For the Pertuzumab/Trastuzumab mixture chromatogram, the Pertuzumab peaks elute first followed by the Trastuzumab peaks. Due to the nature of cation-exchange separation and the net charge difference between Pertuzumab (˜pI 8.7) and Trastuzumab (˜pI 8.9), two main peaks, or major charged species, are observed in the Pertuzumab/Trastuzumab mixture. In contrast, the SEC assay separates based on the hydrodynamic size of the molecule and show only one main peak due to the size similarity between Pertuzumab and Trastuzumab. The charged regions of each molecule appear to overlap with each other in the Pertuzumab/Trastuzumab mixture. Specifically, the Pertuzumab basic variants expected to elute at approximately 32 minutes and at 35 minutes appear to overlap with the main peak of Trastuzumab (FIGS. 8 & 9). Furthermore, the acidic variants of Trastuzumab expected to elute before the Trastuzumab main peak co-elute with the Pertuzumab basic variants and main peak. Despite the overlapping peak regions, the Pertuzumab/Trastuzumab mixture exhibited comparable chromatographic peak profiles before and after storage in IV saline bags for 24 hours at 30° C.

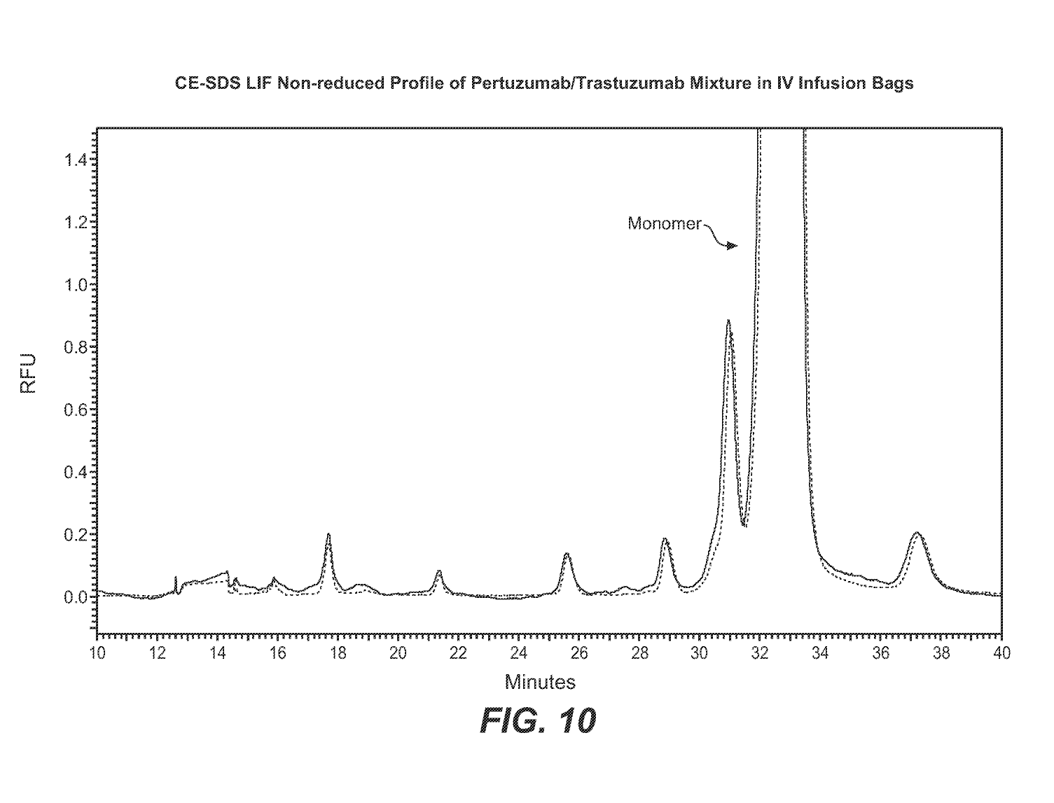

The Pertuzumab/Trastuzumab mixture, Pertuzumab alone, and Trastuzumab alone samples were also assayed on CE-SDS LIF under non-reduced conditions after storage for 24 hours at 30° C. The Pertuzumab/Trastuzumab mixture showed consistent peak profiles with no observable changes after storage compared to the starting material (FIGS. 10 & 11). A very slight baseline level variation attributed to noise is also observed and does not impact peak area. Similar to SEC, the non-reduced Pertuzumab/Trastuzumab mixture showed only one superimposed monomer constituting both the Pertuzumab and Trastuzumab main species. The Pertuzumab and Trastuzumab alone samples showed no changes at T0 compared to T24. However, individual molecular attributes, e.g. fragment peak level and species, between Pertuzumab/Trastuzumab mixture, Pertuzumab alone, and Trastuzumab alone was observed as expected.

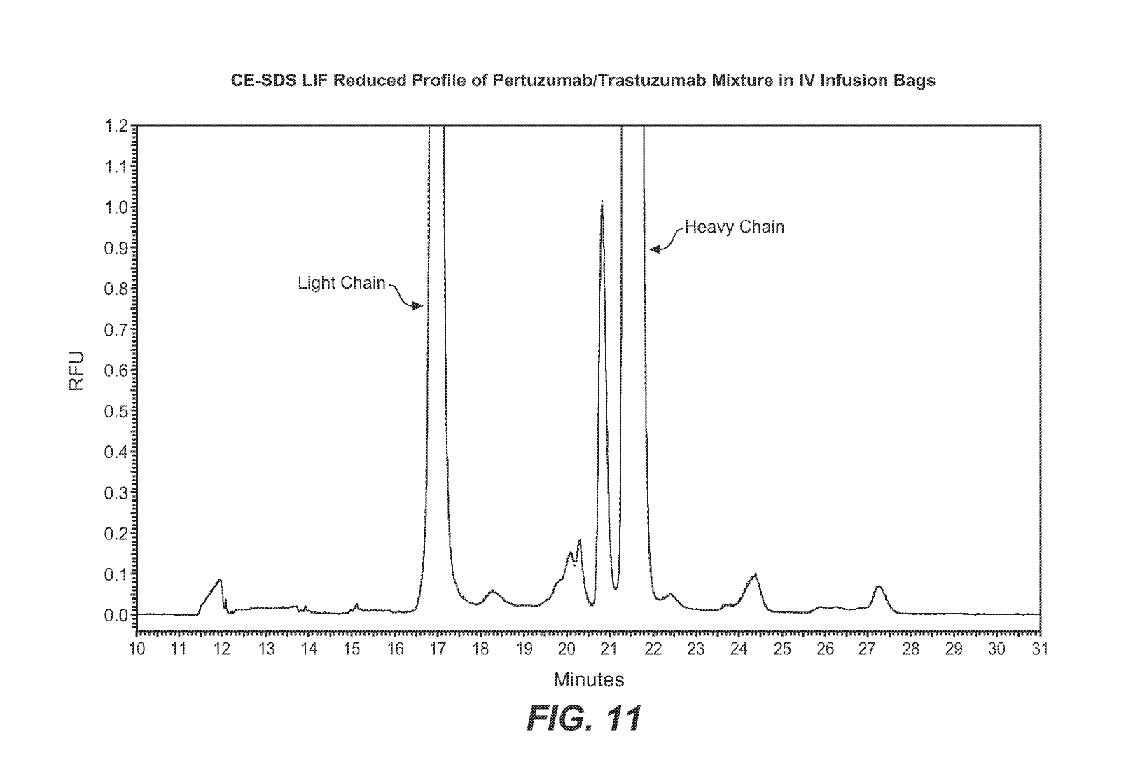

Two major peaks known as the light chain (LC) and heavy chain (HC) are detected at 17 and 21.5 minutes, respectively, when Pertuzumab/Trastuzumab mixture, Pertuzumab alone, and Trastuzumab alone was run on CE-SDS LIF reduced with DTT (FIG. 11). No increase in fragmentation or concomitant decrease in the LC and HC was seen post storage at 30° C. for the Pertuzumab/Trastuzumab mixture. Furthermore, no detectable peak profile differences were noticed in the Pertuzumab and Trastuzumab alone samples post storage.

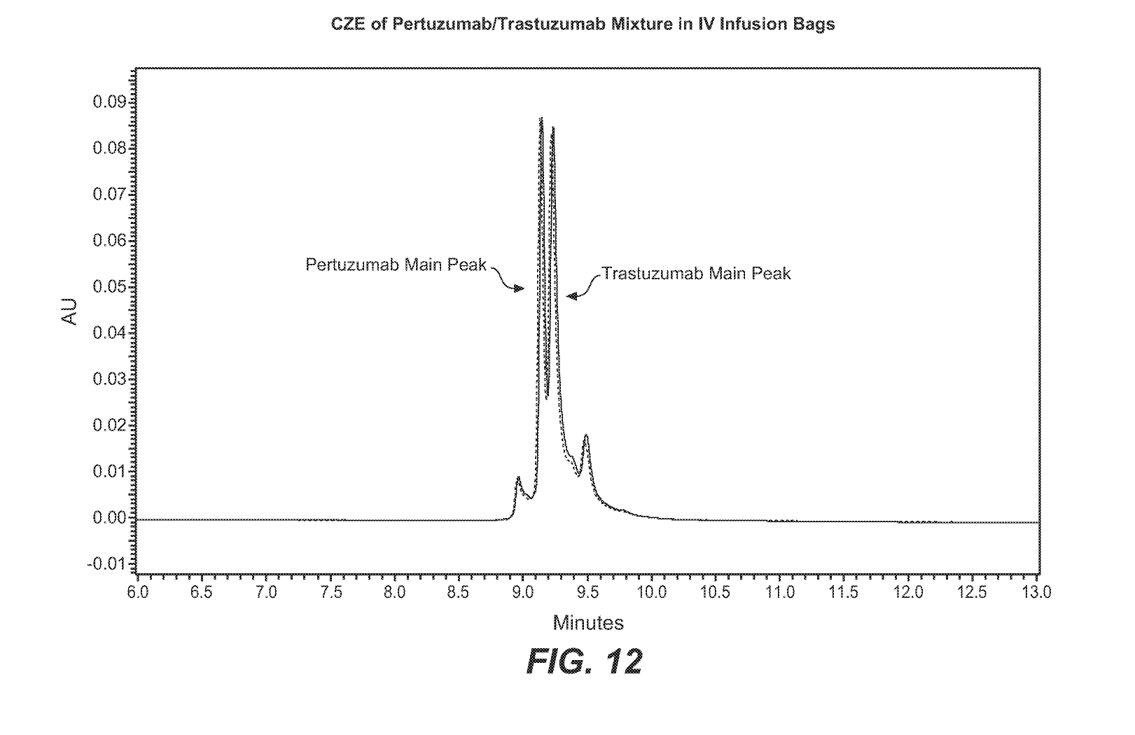

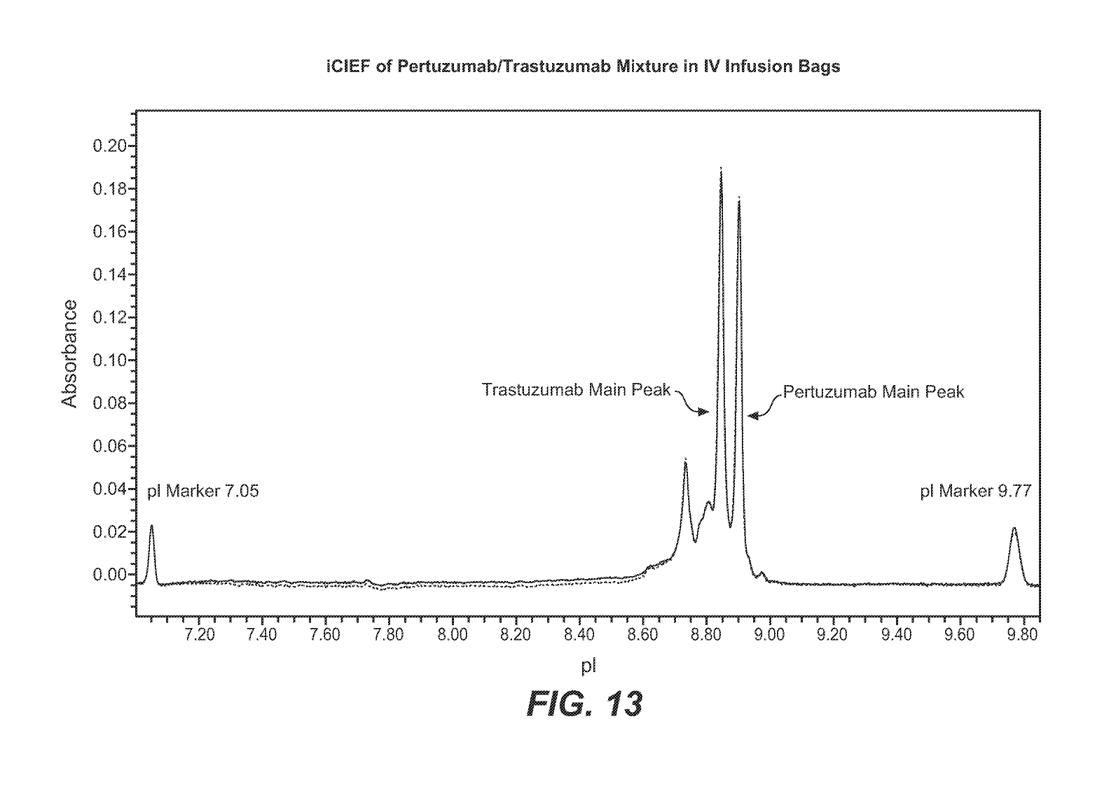

The charge separation assays CZE and iCIEF show comparable peak profiles for the Pertuzumab/Trastuzumab mixture after storage at 30° C. (FIGS. 12 & 13). The Pertuzumab and Trastuzumab alone when compared to their respective T0 also showed consistent peak profiles with no changes after storage. Furthermore, the presence of various minor species was also observed, although no new peaks were detected upon dilution in the IV bag saline solution. As seen in the charge based IEC assay, two main peaks flanked by smaller overlapping peaks can be detected and was attributed to the difference in the molecular pI.

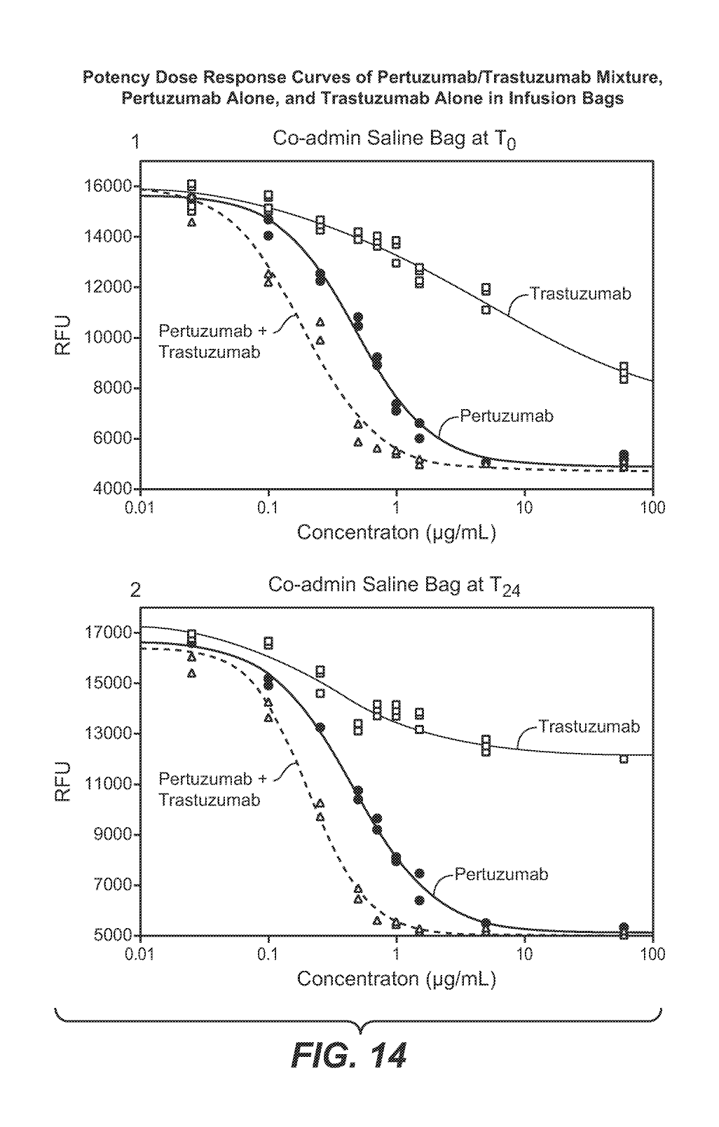

The potency results based on comparison of the dose response curve showed no impact on the potency of the Pertuzumab/Trastuzumab mixture stored at 30° C. for 24 hours compared to its corresponding T0 dose response curve (FIG. 14). The Trastuzumab alone showed little activity in the Pertuzumab potency assay. The Pertuzumab/Trastuzumab mixture dose response curve compared to the dose response curve of Pertuzumab or Trastuzumab alone showed that lower doses of the Pertuzumab/Trastuzumab mixture were needed to inhibit the growth of cells as compared to Pertuzumab alone, suggesting there may be an additive or synergistic effect on the inhibition of cell proliferation for the mixture.

Dose II: 1560 mg Total Pertuzumab/Trastuzumab Mixture (840 mg Pertuzumab and 720 mg Trastuzumab)

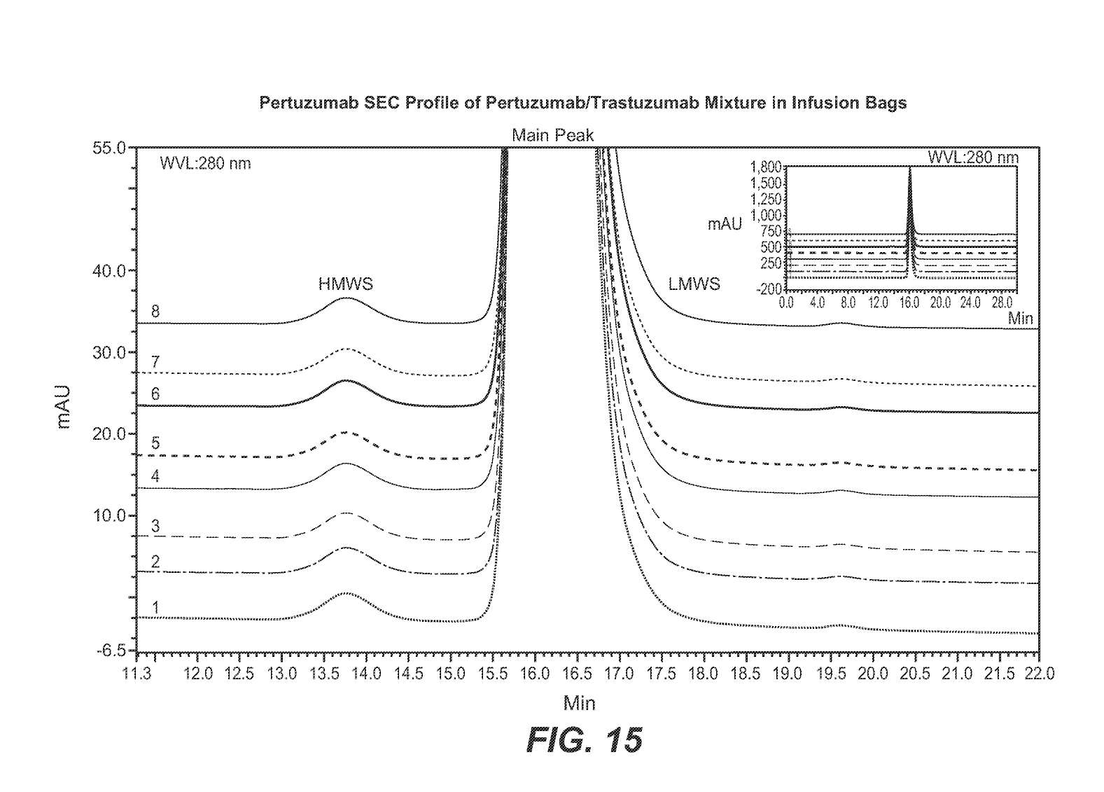

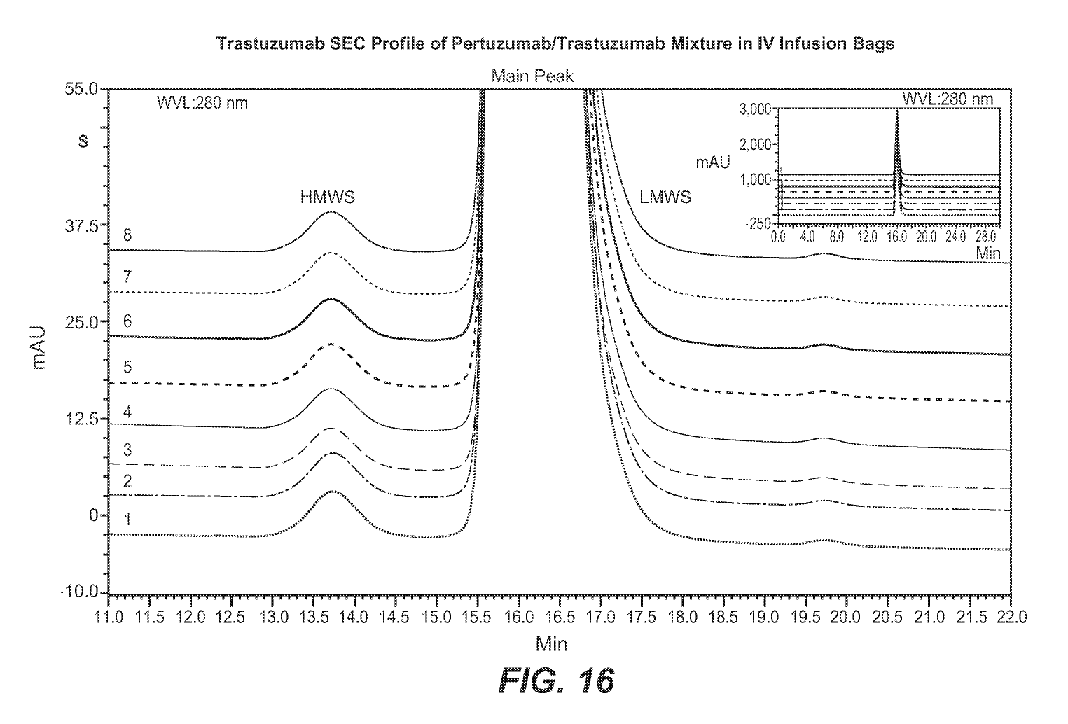

In addition to the dose I study at 840 mg total mAb, a higher dose of 1560 mg mixture (840 mg Pertuzumab and 720 mg Trastuzumab), and their individual drug product controls (840 mg Pertuzumab alone and 720 mg Trastuzumab alone) was selected to investigate the impact of diluting these three mAb types in PO or PVC IV infusion bags at 5° C. or 30° C. for up to 24 hours. The product quality of these IV infusion bags before and after storage was assessed by CAC, UV-spec scan (concentration and turbidity), and HIAC-ROYCO™ are summarized in Table 3 and SEC and IEC are shown in FIGS. 15-18.

TABLE 3
Dose II 1560 mg: Stability data for Pertuzumab/Trastuzumab mixture, Pertuzumab, or
Trastuzumab in 0.9% saline PO IV infusion bags (n = 1 for control; n = 2 for mixture)

| | | | | | | | | Light Obscuration | |
|---|---|---|---|---|---|---|---|---|---|
| | | | | | | | | total | total |
| | IV bag | Amount | Temp | Timepoint | CAC^{a} | Conc. | Turbidity | particles | particles |
| Sample | type | mg | ° C. | Hour(s) | liquid | mg/mL | AU | ≧10 um/mL | ≧25 um/mL |
| pertuzumab/ | PO | 1560 | 5 | 0 | CL, CO | 4.9 | 0.013 | 20 | 1 |
| trastuzumab | | | | 24 | CL, CO | 4.7 | 0.016 | 15 | 1 |
| mixture | | 1560 | 30 | 0 | CL, CO | 5.0 | 0.014 | 8 | 0 |
| | | | | 24 | CL, CO | 4.9 | 0.018 | 18 | 0 |
| pertuzumab | PO | 840 | 5 | 0 | CL, CO | 2.9 | 0.007 | 1 | 0 |
| | | | | 24 | CL, CO | 2.9 | 0.005 | 6 | 0 |
| | | 840 | 30 | 0 | CL, CO | 2.8 | 0.006 | 6 | 0 |
| | | | | 24 | CL, CO | 2.8 | 0.004 | 9 | 0 |
| trastuzumab | PO | 720 | 5 | 0 | CL, CO | 2.6 | 0.004 | 4 | 0 |
| | | | | 24 | CL, CO | 2.6 | 0.005 | 13 | 0 |
| | | 720 | 30 | 0 | CL, CO | 2.5 | 0.007 | 19 | 0 |
| | | | | 24 | CL, CO | 2.5 | 0.004 | 14 | 0 |
| pertuzumab/ | PVC | 1560 | 5 | 0 | CL, CO | 4.9 | 0.016 | 18 | 0 |
| trastuzumab | | | | 24 | CL, CO | 4.7 | 0.015 | 18 | 0 |
| mixture | | 1560 | 30 | 0 | CL, CO | 4.8 | 0.016 | 24 | 0 |
| | | | | 24 | CL, CO | 4.8 | 0.012 | 17 | 0 |
| pertuzumab | PVC | 840 | 5 | 0 | CL, CO | 2.9 | 0.006 | 13 | 0 |
| | | | | 24 | CL, CO | 2.7 | 0.004 | 10 | 0 |
| | | 840 | 30 | 0 | CL, CO | 2.8 | 0.006 | 6 | 0 |
| | | | | 24 | CL, CO | 2.8 | 0.006 | 11 | 0 |
| trastuzumab | PVC | 720 | 5 | 0 | CL, CO | 2.5 | 0.007 | 7 | 0 |
| | | | | 24 | CL, CO | 2.5 | 0.004 | 9 | 0 |
| | | 720 | 30 | 0 | CL, CO | 2.5 | 0.003 | 18 | 0 |
| | | | | 24 | CL, CO | 2.5 | 0.005 | 19 | 0 |

^{a}Color, Appearance and Clarity: CL = clear; SOPL = slightly opalescent, CO = colorless.

Two PO or PVC TV infusion bags each were prepared for the Pertuzumab/Trastuzumab mixture condition while only one IV infusion bag was prepared for the Pertuzumab and Trastuzumab alone samples.

Particulates from these bags were determined by visual observation, turbidity, and HIAC-Royco measurements. All samples appeared clear and colorless after storage at 5° C. or 30° C. for up to 24 hours. No visible particulate matter was observed and there was no significant change in the turbidity post storage. For the Pertuzumab/ Trastuzumab mixture, Pertuzumab alone, and Trastuzumab alone, the HIAC-Royco showed comparable particle values before and after storage, with zero to 10 particles increase per milliliter at ≧10 μm and zero particle increase per milliliter at ≧25 μm for both PO and PVC IV infusion bags stored at either 5° C. or 30° C. Similarly, the Pertuzumab and Trastuzumab alone samples also exhibited no significant particle differences before and after storage in PO or PVC IV infusion bags. For all three sample types, the UV-spec scan showed no changes beyond normal assay variability in protein concentration, indicating the absence of protein adsorption or precipitation in the IV infusion bags between T0 and T24 hours at 5° C. or 30° C. storage.

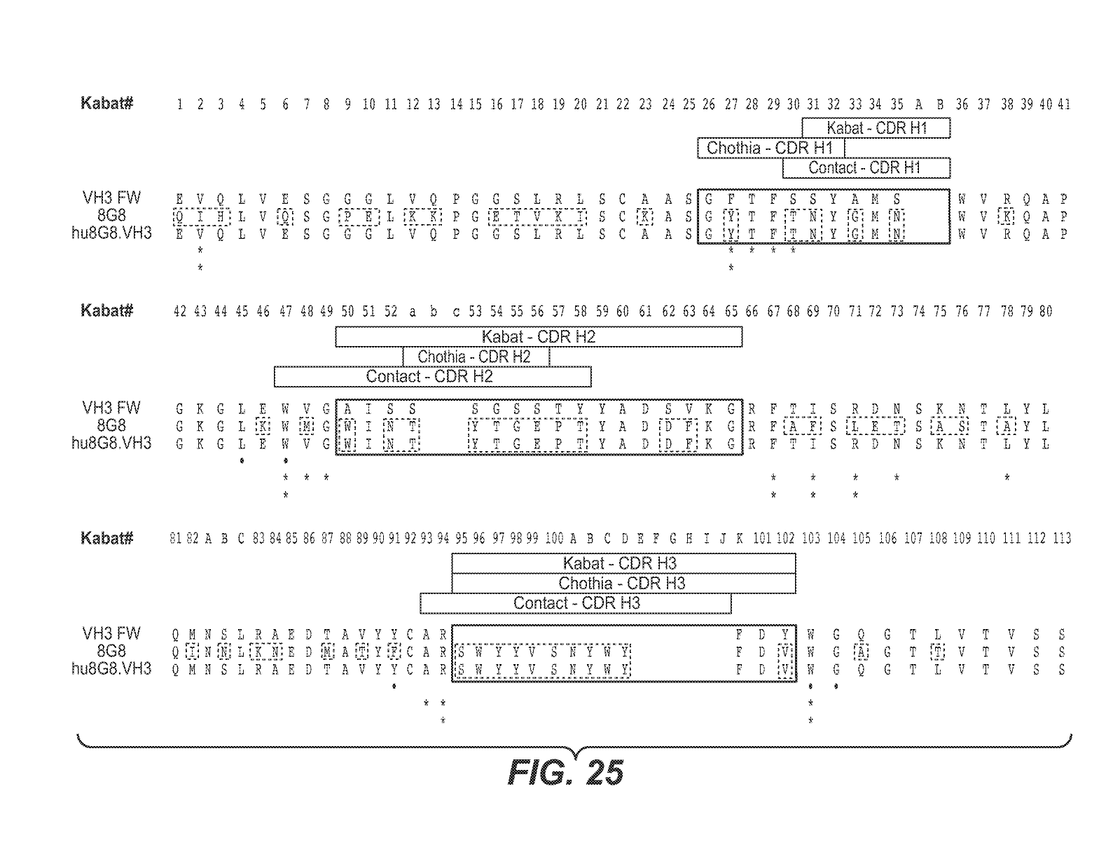

The Pertuzumab/Trastuzumab mixture, Pertuzumab alone, and Trastuzumab alone samples were analyzed using Pertuzumab or Trastuzumab specific SEC and IEC methods to assess their physical and chemical stability, respectively, as previously described. For the Pertuzumab/Trastuzumab mixture, no changes in SEC were observed in the chromatographic profiles between the T0 and the T24 hour samples at 5° C. or 30° C. in either PO or PVC IV infusion bags (FIGS. 24 and 25), similar to the 840 mg mixture dose I results. In addition, no increase or decrease in the high molecular weight species (HMWS), main peak, and low molecular weight species (LMWS) was observed, which indicates a stable dosing solution at the upper ranges of protein content in 0.9% saline. Likewise, Pertuzumab alone and Trastuzumab alone samples also showed no changes after storage in the IV infusion bags.

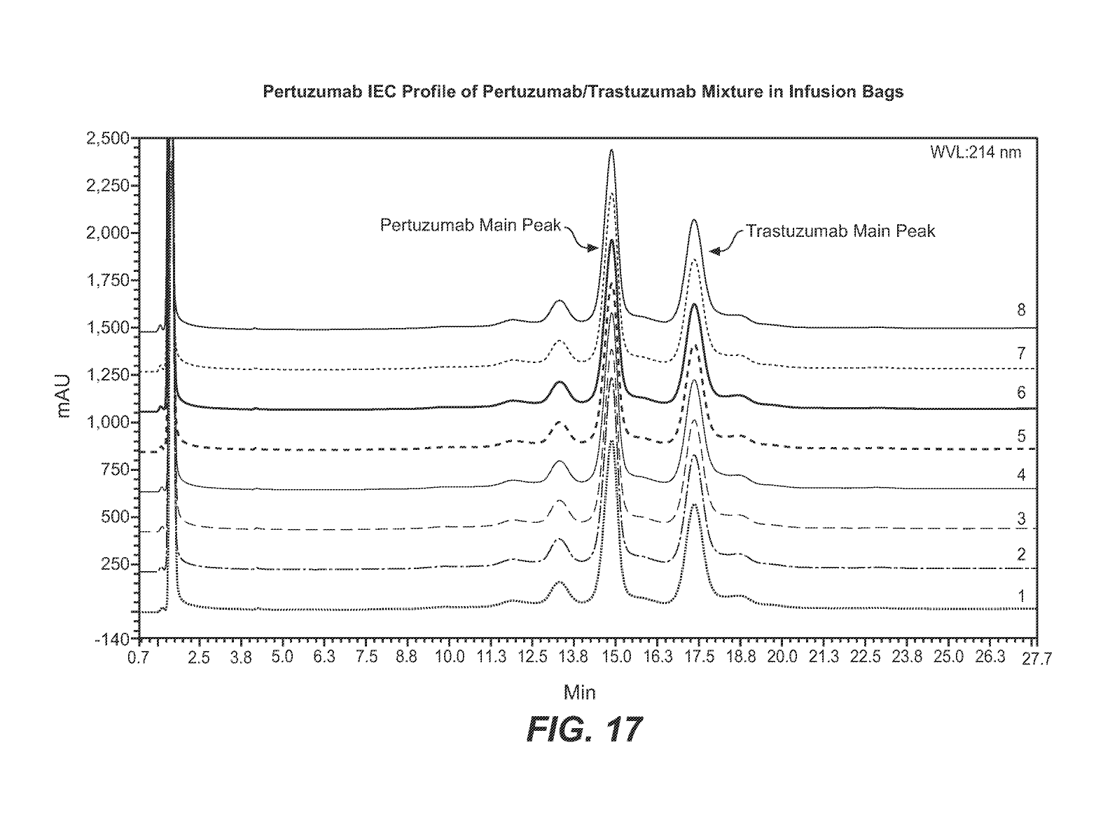

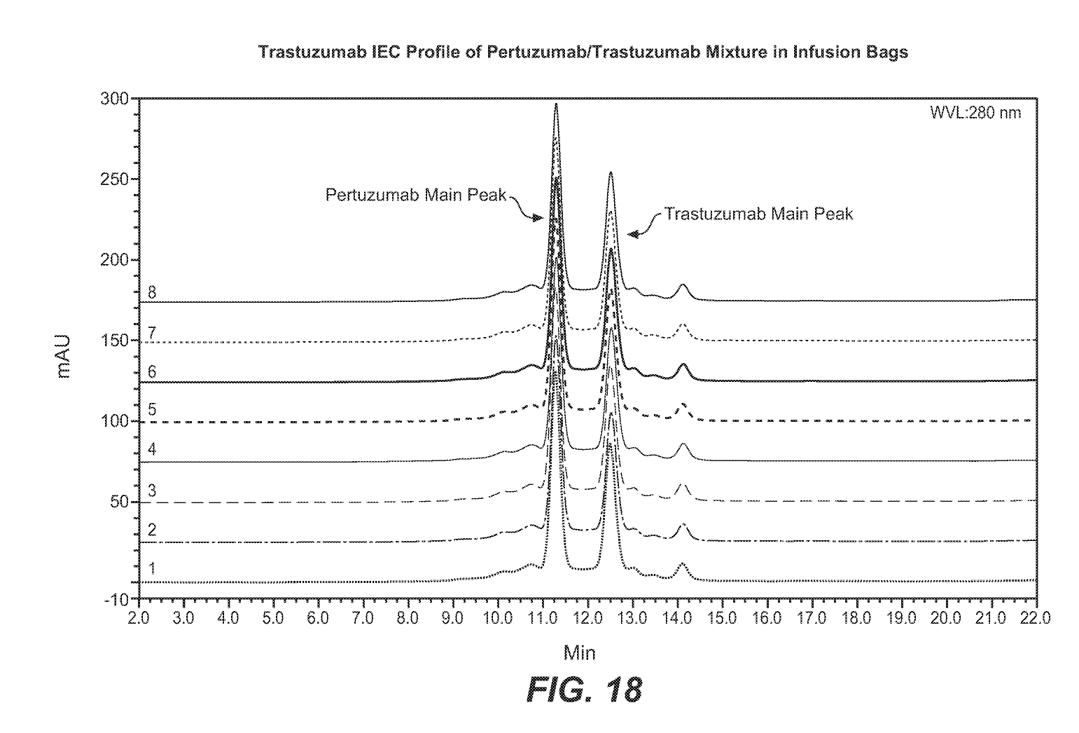

IEC analysis, using both the Pertuzumab or Trastuzumab specific methods, of the Pertuzumab/Trastuzumab mixture was used to assess chemical stability and showed comparable charge variant peak profiles with no observed changes relative to the initial time point after exposure to 5° C. or 30° C. in the PO or PVC IV infusion bags (FIGS. 17 and 18). Although a significant overlap of the charge variant species of the two mAbs were observed, these peaks species were not impacted from the increase in the mAb content of the IV infusion bag. Pertuzumab alone or Trastuzumab alone samples in PO or PVC IV infusion bags showed no changes before and after exposure to 5° C. or 30° C. These results are consistent with the 840 mg dose I study.

### Conclusion

All physicochemical assays indicate no significant changes in the mixtures (up to 840 mg Pertuzumab and 720 mg Trastuzumab for a 1560 mg total dose) or in the individual Pertuzumab (up to 840 mg) and Trastuzumab (up to 720 mg) IV infusion bags (PO or PVC) for T0 to T24 hours at 5° C. or 30° C. Furthermore, the potency of the mixture (up to 840 mg) and the individual mAbs before and after storage were comparable. No differences were observed in the IV bags that contained the admixture of Pertuzumab and Trastuzumab when compared to the individual mAb components in IV bags over the course of this study. The current study also demonstrates that many of the assays used to measure the individual mAbs were sufficient to qualitatively characterize the admixture.

### EXAMPLE 2

### Co-Administration of Pertuzumab and Trastuzumab, and Combination Therapy With VinoreIbine

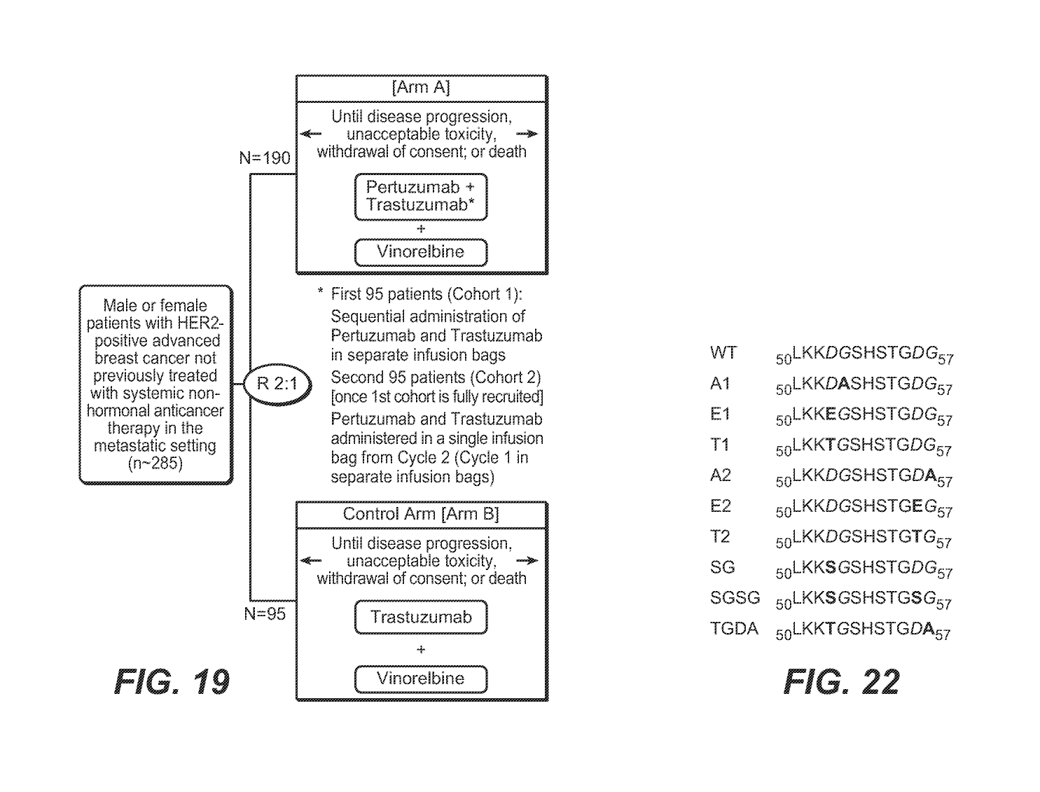

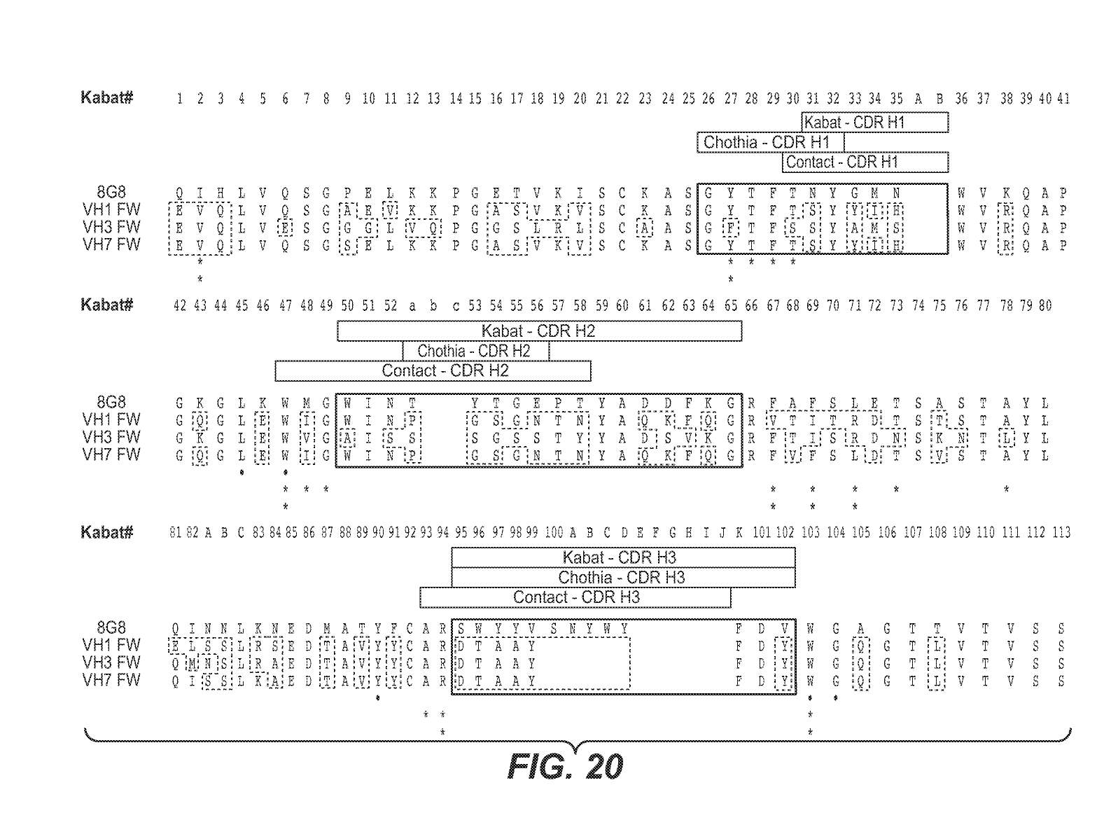

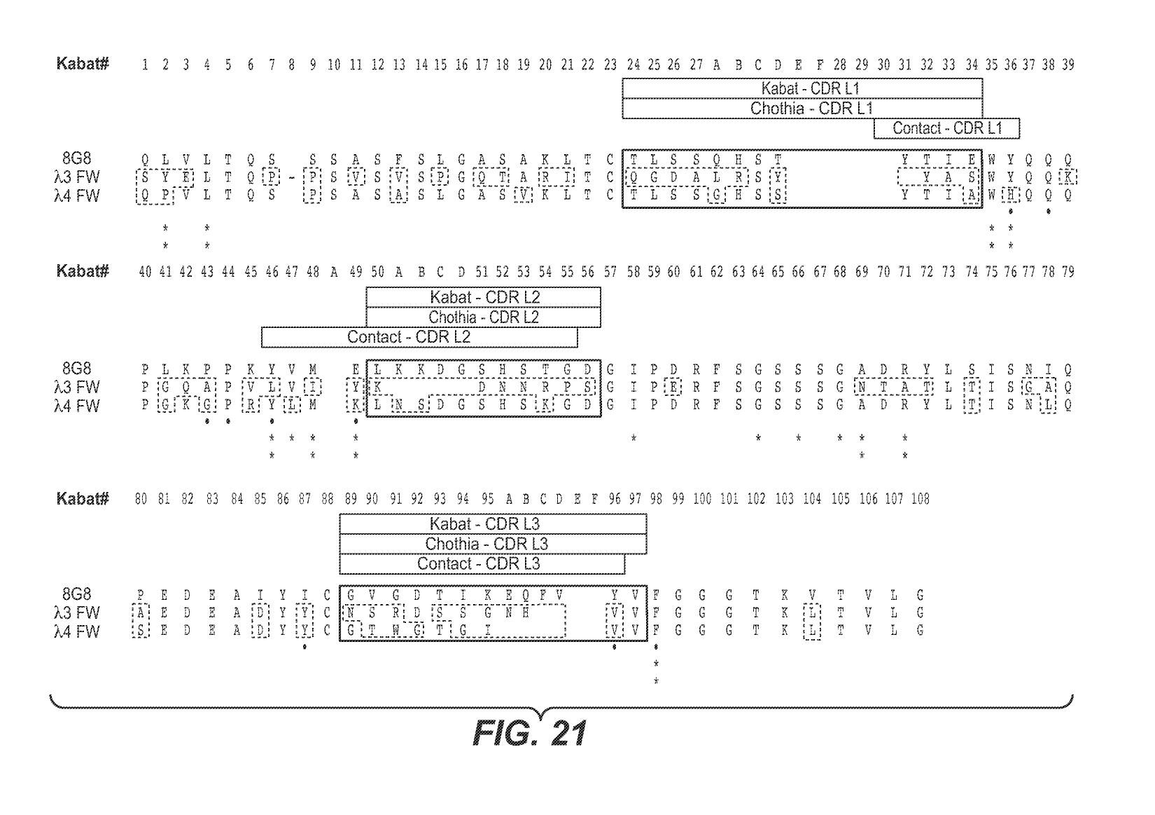

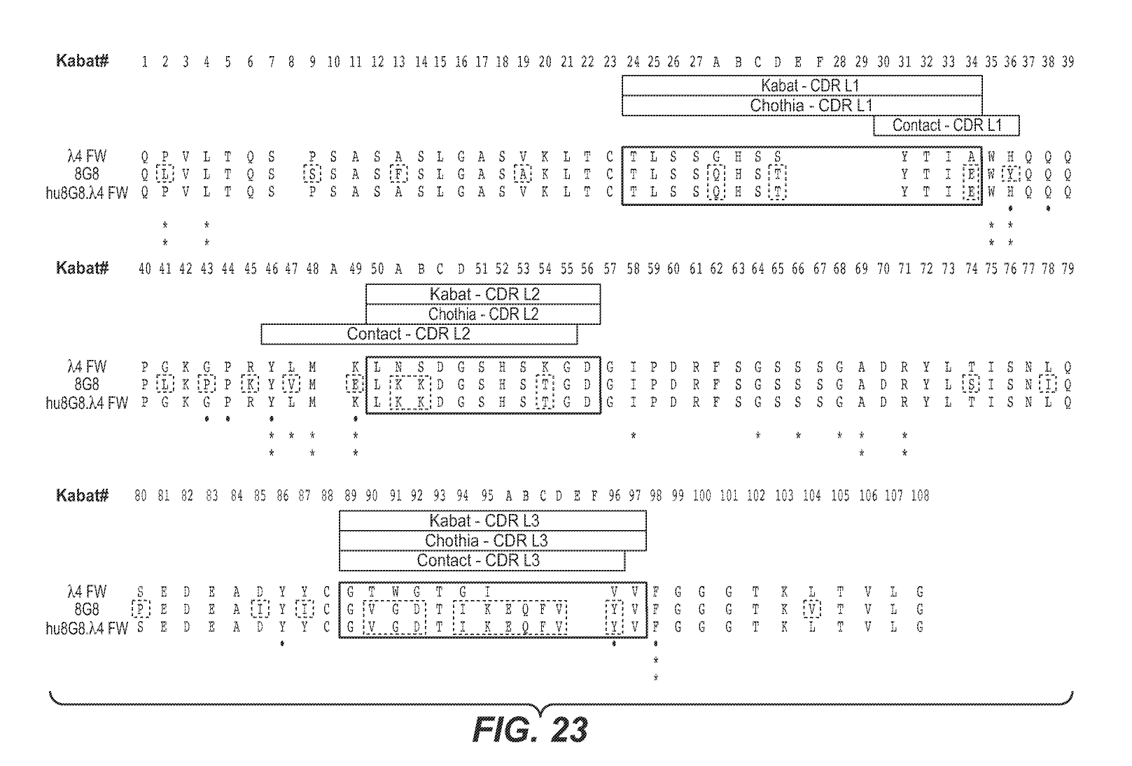

This is a randomized, two-arm, open-label, multicenter Phase II trial to evaluate Pertuzumab in patients with HER2-positive advanced breast cancer (metastatic or locally advanced) who have not previously received systemic non-hormonal anticancer therapy in the metastatic setting. The study design is shown in FIG. 19.

Patients are randomly assigned in a 2:1 ratio to one of two treatment arms:
Pertuzumab given in combination with Trastuzumab and vinorelbine (Arm A)
Trastuzumab and vinorelbine (control arm Arm B)
   Arm A will consist of two cohorts as follows:

Cohort 1: (first 95 patients): Pertuzumab and Trastuzumab administered sequentially in separate infusion bags, followed by vinorelbine. Patients will receive Pertuzumab followed by Trastuzumab sequentially in separate infusion bags, followed by vinorelbine.

### Pertuzumab (IV Infusion)

Administered on Day 1 of the first treatment cycle as a loading dose of 840 mg, followed by 420 mg on Day 1 of each subsequent 3 weekly cycle.

Initial infusions of Pertuzumab will be administered over 90 (±10) minutes and patients observed for at least 30 minutes from the end of infusion for infusion-related symptoms such as fever, chills etc. Interruption or slowing of the infusion may reduce such symptoms. If the infusion is well tolerated, subsequent infusions may be administered over 30 (±10) minutes with patients observed for a further 30 minutes.

### Trastuzumab (IV Infusion)

Day 1 of the first treatment cycle as a loading dose of 8 mg/kg, followed by 6 mg/kg on Day 1 of each subsequent 3 weekly cycle; to be administered in line with product labeling.

### Vinorelbine (IV Infusion After Trastuzumab)

Day 1 and Day 8 of the first treatment cycle at a dose of 25 mg/m² followed by 30-35 mg/m² on Day 1 and Day 8 of each subsequent 3 weekly cycle; to be administered in line with product labeling.

Cohort 2: The second 95 patients will receive Pertuzumab and Trastuzumab administered together in a single infusion bag from Cycle 2 onwards, followed by vinorelbine.

### Cycle 1 Dosing

For the first cycle of treatment, Pertuzumab and Trastuzumab will be administered in separate infusion bags as described for Cohort 1.

Vinorelbine will be administered after Pertuzumab and Trastuzumab as described for Cohort 1.

### Subsequent Cycle Dosing

If administration of all three drugs was well tolerated in Cycle 1, then on Day 1 of each subsequent 3 weekly treatment cycle, Pertuzumab 420 mg and Trastuzumab 6 mg/kg will be given together in a single infusion bag.

The first combined infusion of Pertuzumab and Trastuzumab should be administered over 90 (±10) minutes with cardiac monitoring and close observation for infusion-associated reactions during the procedure, followed by a 60 minute observation period. If this first combined infusion is well tolerated, subsequent combined infusions can be administered over 60 (±10) minutes followed by a 30 minute observation period with cardiac monitoring.

Vinorelbine will be administered after Pertuzumab and Trastuzumab as described for Cohort 1.

### Control Arm—Arm B

A total of 95 patients will be randomized to arm B.

### Trastuzumab (IV Infusion)

Day 1 of the first treatment cycle as a loading dose of 8 mg/kg, followed by 6 mg/kg on Day 1 of each subsequent 3 weekly cycle; to be administered in line with product labeling.

### Vinorelbine (IV Infusion After Trastuzumab)

Day 1 and Day 8 of the first treatment cycle at a dose of 25 mg/m² followed by 30-35 mg/m² on Day 1 and Day 8 of each subsequent 3 weekly cycle; to be administered in line with product labeling.

### Efficacy Outcomes:

### Primary

To compare objective overall response rates (ORR) assessed by a blinded independent review committee (IRC) of Pertuzumab given in combination with Trastuzumab and vinorelbine versus Trastuzumab and vinorelbine

### Secondary

Within the Pertuzumab treatment group to compare the efficacy and safety of Pertuzumab and Trastuzumab administered together in a single infusion bag versus conventional sequential administration in separate infusion bags
To compare Pertuzumab given in combination with Trastuzumab and vinorelbine versus Trastuzumab and vinorelbine with respect to:
   ORR assessed by the Investigator
   Time to response assessed by IRC and Investigator
   Duration of response assessed by IRC and Investigator
   Progression free survival (PFS)
   Time to progression (TTP)
   Overall survival (OS)
   Safety and tolerability
   Quality of life (EQ-5D and FACT-B questionnaires)

### Inclusion Criteria

Patients must meet the following criteria to be eligible for this study according to the timing of the Schedule of Assessments:
1. Female or male patients aged 18 years or older
2. Histologically or cytologically confirmed and documented adenocarcinoma of the breast with metastatic or locally advanced disease not amenable to curative resection
3. HER2-positive (defined as either immunohistochemistry (IHC) 3+ or in situ hybridization (ISH) positive) as assessed by local laboratory on primary or metastatic tumor (ISH positivity is defined as a ratio of 2.0 or greater for the number of HER2 gene copies to the number of signals for CEP17, or for single probe tests, a HER2 gene count greater than 4).
4. At least one measurable lesion and/or non-measurable disease evaluable according to Response Evaluation Criteria In Solid Tumors (RECIST) version 1.1
5. ECOG performance status 0 or 1
6. Left ventricular ejection fraction (LVEF) of at least 50%
7. Negative pregnancy test in women of childbearing potential (premenopausal or less than 12 months of amenorrhea post-menopause, and who have not undergone surgical sterilization)
8. For women of childbearing potential who are sexually active, agreement to use a highly-effective, non-hormonal form of contraception or two effective forms of non-hormonal contraception during and for at least 6 months post study treatment
9. Fertile males willing and able to use effective non-hormonal means of contraception (barrier method of contraception in conjunction with spermicidal jelly, or surgical sterilization) during and for at least 6 months post-study treatment
10. Life expectancy of at least 12 weeks

### Exclusion Criteria

Patients who meet any of the following exclusion criteria will not be eligible for this study:
1. Previous systemic non-hormonal anticancer therapy in the metastatic or locally advanced breast cancer setting
2. Previous approved or investigative anti-HER2 agents in any breast cancer treatment setting, except Trastuzumab in the adjuvant or neoadjuvant setting
3. Disease progression while receiving Trastuzumab in the adjuvant or neoadjuvant setting
4. Disease-free interval from completion of adjuvant or neo-adjuvant systemic non-hormonal treatment to recurrent disease of less than 6 months
5. History of persistent grade 2 or higher (NCI-CTC, Version 4.0) hematological toxicity resulting from previous adjuvant or neoadjuvant therapy
6. Radiographic evidence of central nervous system (CNS) metastases as assessed by CT or MRI
7. Current peripheral neuropathy of grade 3 or greater (NCI-CTC, Version 4.0)
8. History of other malignancy within the last 5 years, except for carcinoma in situ of the cervix or basal cell carcinoma
9. Serious uncontrolled concomitant disease that would contraindicate the use of any of the investigational drugs used in this study or that would put the patient at high risk for treatment related complications
10. Inadequate organ function, evidenced by the following laboratory results:
   Absolute neutrophil count <1,500 cells/mm³
   Platelet count <100,000 cells/mm³
   Hemoglobin <9 g/dL
   Total bilirubin greater than upper limit of normal (ULN) (unless the patient has documented Gilbert's syndrome)
   AST (SGOT) or ALT (SGPT) >2.5×ULN
   AST (SGOT) or ALT (SGPT) >1.5×ULN with concurrent serum alkaline phosphatase >2.5×ULN; Serum alkaline phosphatase may be >2.5×ULN only if bone metastases are present and AST (SGOT) and ALT (SGPT) <1.5×ULN Serum creatinine >2.0 mg/dL or 177 μmol/L
   International normalized ratio (INR) and activated partial thromboplastin time or partial thromboplastin time (aPTT or PTT) >1.5×ULN (unless on therapeutic coagulation)
11. Uncontrolled hypertension (systolic >150 mm Hg and/or diastolic >100 mm Hg) or clinically significant (i.e. active) cardiovascular disease: cerebrovascular accident (CVA)/stroke or myocardial infarction within 6 months prior to first study medication, unstable angina, congestive heart failure (CHF) of New York Heart Association (NYHA) grade II or higher, or serious cardiac arrhythmia requiring medication
12. Current known infection with HIV, HBV, or HCV
13. Dyspnea at rest due to complications of advanced malignancy, or other disease requiring continuous oxygen therapy
14. Major surgical procedure or significant traumatic injury within 28 days prior to randomization or anticipation of need for major surgery during the course of study treatment
15. Receipt of intravenous (IV) antibiotics for infection within 14 days prior to randomization
16. Current chronic daily treatment with corticosteroids (dose equivalent to or greater than 10 mg/day methylprednisolone), excluding inhaled steroids
17. Known hypersensitivity to any of the study medications or to excipients of recombinant human or humanized antibodies
18. History of receiving any investigational treatment within 28 days prior to randomization
19. Concurrent participation in any clinical trial

It is anticipated that the treatment herein will demonstrate the safety and efficacy of co-administration of Pertuzmab and Trastuzumab from the same intravenous (IV) bag to patients with HER2-positive cancer (exemplified by HER2-positive breast cancer), as well as the safety and efficacy of Pertuzumab in combination in vinorelbine according to any one or more of the primary or secondary efficacy outcomes above.

### EXAMPLE 3

### Co-Administration of an Anti-Complex 1 and Anti-gH Antibody From a Single IV Bag

For the treatment of congenital cytomegalovirus (CMV) infection two anti-CMV monoclonal antibodies are co-delivered to a patient in need, from the same N bag. In particular, an anti-Complex 1 and an anti-gH antibody are formulated and packaged separately and then filled into the same 250-mL PVC or polyolefin (PO) infusion bag in normal saline (0.9% NaCl) solution. In particular, each antibody is formulated at 20 mg/mL in 20 mM histidine acetate, 240 mM sucrose, 0.02% polysorbate 20, pH 5.5. The treatment is performed with an administration set, including the IV bag, an infusion tube with filter and a catheter. It is important to ensure that the antibody mixture does not adbsorb to any components of the administration set, including the bag, line and catheter.

For clinical trial, the two antibodies are co-administered by IV infusion using IV bags. Studies to evaluate the stability and compatibility of the two antibodies in the IV bag for con-administration are conducted in a three-arm study, using the two antibodies alone and in combination. Characteristics analyzed to monitor the stability of the two antibodies in the same IV bag include physical appearance, pH, protein concentration, size heterogeneity, charge heterogeneity, potency, particulate analysis, and container/closure integrity. Stability and compatibility for co-administration from the same IV bag are demonstrated for up to 8 hours at room temperature.

The assays to assess stability are as described in Example 1, including CAC, turbidity assays, HIAC Royco™, icIEF, and SEC.

The IV bag solutions containing both monoclonal antibodies use total absorbance units (AU) to monitor concentration, instead of mg/mL.

Potency is tested in an assay measuring the prevention of CMV virus infection of epithelial (e.g. ARPE) cells.

In a pilot study the following results were obtained:

CAC (visual): no particles seen in comix after 24 hours at 30° C. when viewed <1 hours after passage through administration set.

Turbidity: no increase in any samples, no sample greater than 0.01 AU.

HIAC: passes USP requirements for large volume injections: 10 particles/mL≧10 μM, 3 particles/mL≧25 μM.

Strength (UV-Vis): Recovery 88-112%

SEC: no change between t=0 and 24 hr 30° C.

What is claimed is:

1. An article of manufacture containing a stable liquid mixture of more than one monoclonal antibody, formulated separately, suitable for intravenous administration to a patient in need.

2. The article of manufacture or claim 1, which is an intravenous (IV) bag.

3. The article of manufacture of claim 2, wherein at least one antibody is a naked antibody.

4. The article of manufacture of claim 3, wherein all antibodies are naked antibodies.

5. The article of manufacture of claim 2, wherein at least one antibody is an anti-cancer antibody.

6. The article of manufacture of claim 5, wherein all antibodies are anti-cancer antibodies.

7. The article of manufacture of claim 2, wherein at least one antibody is an anti-viral antibody.

8. The article of manufacture of claim 7, wherein all antibodies are anti-viral antibodies.

9. The article of manufacture of claim 2, wherein at least one antibody is infused for at least about 90 minutes when administered individually.

10. The article of manufacture of claim 2, wherein at least one antibody is infused for at least about 120 minutes when administered individually.

11. The article of manufacture of claim 2, wherein at least one antibody is infused for about 90 minutes to about 10 hours when administered individually.

12. The article of manufacture of claim 2, wherein each antibody present in the mixture is infused for at least about 120 minutes when administered individually.

13. The article of manufacture of claim 2, wherein the IV bag contains two antibodies.

14. The article of manufacture of claim 2, wherein the mixture is stable for at least about 4 to 6 hours at 2 to 8° C. or 15 to 30° C.

15. The article of manufacture of claim 2, wherein the mixture is stable for at least about 8 hours at 2 to 8° C. or 15 to 30° C.

16. The article of manufacture of claim 2, wherein the mixture is stable for at least about 12 hours at 2 to 8° C. or 15 to 30° C.

17. The article of manufacture of claim 2, wherein the mixture is stable for at least about 24 hours at 2 to 5° C. or 15 to 30° C.

18. The article of manufacture of claim 14, wherein stability is measured at 5° C. or at 30° C.

19. The article of manufacture of claim 2, wherein the mixture is in a saline solution.

20. The article of manufacture of claim 2, wherein the mixture is in a dextrose solution.

21. The article of manufacture of claim 19, wherein the saline solution comprises about 0.9% NaCl or about 0.45% NaCl.

22. The article of manufacture of claim 2, wherein the IV bag is a polyolefin or polyvinyl chloride infusion bag.

23. The article of manufacture of claim 22, wherein the polyolefin is polypropylene or polyethylene.

24. The article of manufacture of claim 1, wherein stability has been evaluated by an assay selected from the group consisting of: color, appearance and clarity (CAC), concentration and turbidity analysis, particulate analysis, size exclusion chromatography (SEC), ion-exchange chromatography (MC), reverse phase HPL, hydrophobic interaction chromatography, HIAC-Royco, capillary zone electrophoresis (CZE), image capillary isoelectric focusing (iCIEF), and potency assay.

25. The article of manufacture of claim 2, wherein at least one monoclonal antibody binds to an antigen selected from the group consisting of EGFR, HER2, HER3, HER4, CD20, CD22, IL-8, CD40, CD11a, IgE, STIgMA, CD18, Apo-2 receptor, TNF-α, Tissue Factor (TF), human α₄-β₇ integrin, CD3, CD25, CD52, CD33, CD38, tac, Fc receptor, carcinoembryonic antigen (CEA), EpCAM, GpIIb/IIIa, RSV, CMV, HIV, Hep B, αvβ3, IL-17A, IL-17A/F, GD3 ganglioside; and human leukocyte antigen (HLA).

26. The article of manufacture of claim 25, wherein at least monoclonal antibody binds to HER2.

27. The article of manufacture of claim 25, wherein at least two monoclonal antibodies bind to HER2.

28. The article of manufacture of claim 27, wherein the IV bag contains a mixture of Trastuzumab and Pertuzumab.

29. The article of manufacture of claim 2, wherein at least one monoclonal antibody binds to CMV.

30. The article of manufacture of claim 29, wherein at least two monoclonal antibodies bind to CMV.

31. The article of manufacture of claim 29, wherein at least one monoclonal antibody binds to HCMV Complex I.

32. The article of manufacture of claim 31, wherein at least one monoclonal antibody binds to HCMV gH.

33. The article of manufacture of claim 32, wherein the IV bag contains a mixture of an antibody specifically binding to HCMV gH and an antibody specifically binding to HCMV Complex I.

34. A method for intravenous administration of at least two antibodies and/or antibody-like molecules, wherein said antibodies and/or antibody-like molecules are formulated separately and are administered from a stable liquid mixture contained in a single intravenous (IV) bag.

35. The method of claim 34, wherein at least one antibody is a naked antibody.

36. The method of claim 35, wherein all antibodies are naked antibodies.

37. The method of claim 34, wherein at least one antibody is an anti-cancer antibody.

38. The method of claim 37, wherein at all antibodies are anti-cancer antibodies.

39. The method of claim 34, wherein at least one antibody is an anti-viral antibody.

40. The method of claim 39, wherein all antibodies are anti-viral antibodies.

41. The method of claim 34, wherein at least one antibody is infused for at least about 90 minutes when administered individually.

42. The method of claim 34, wherein at least one antibody is infused for at least about 120 minutes when administered individually.

43. The method of claim 34, wherein at least one antibody is infused for about 90 minutes to about 10 hours when administered individually.

44. The method of claim 34, wherein each antibody present in the mixture is infused for at least about 120 minutes when administered individually.

45. The method of claim 34, wherein the IV bag contains two antibodies.

46. The method of claim 34, wherein the mixture is stable for at least about 4 to 6 hours at 2 to 8° C. or 15 to 30° C.

47. The method of claim 34, wherein the mixture is stable for at least about 8 hours at 2 to 8° C. or 15 to 30° C.

48. The method of claim 34, wherein the mixture is stable for at least about 12 hours at 2 to 8° C. or 15 to 30° C.

49. The method of claim 34, wherein the mixture is stable for at least about 24 hours at 2 to 8° C. or 15 to 30° C.

50. The method of claim 46, wherein stability is measured at 5° C. or at 30° C.

51. The method of claim 34, wherein the mixture is in a saline solution.

52. The method of claim 34, wherein the mixture is in a dextrose solution.

53. The method of claim 52, wherein the saline solution comprises about 0.9% NaCl or about 0.45% NaCl.

54. The method of claim 23, wherein the IV bag is a polyolefin or polyvinyl chloride infusion bag.

55. The method of claim 54, wherein the polyolefin is polypropylene or polyethylene.

56. The method of claim 34, wherein stability has been evaluated by an assay selected from the group consisting of: color, appearance and clarity (CAC), concentration and turbidity analysis, particulate analysis, size exclusion chromatography (SEC), ion-exchange chromatography (IEC), reverse phase HPL, hydrophobic interaction chromatography, HIAC-Royco, capillary zone electrophoresis (CZE), image capillary isoelectric focusing (iCIEF), and potency assay.

57. The method of claim 34, wherein at least one monoclonal antibody binds to an antigen selected from the group consisting of EGFR, HER2, HER3, HER4, CD20, CD22, CD40, CD11a, IgE, CD18, Apo-2 receptor, TNF-α, Tissue Factor (TF), human α₄-β₇ integrin, CD3, CD25, CD52, CD33, CD38, tac, Fc receptor, carcinoembryonic antigen (CEA), EpCAM, GpIIb/IIIa, RSV, CMV, HIV, Hep B, αvβ3, IL-17A, IL-17A/F, IL-17F, GD3 ganglioside; and human leukocyte antigen (HLA).

58. The method of claim 34, wherein at least monoclonal antibody binds to HER2.

59. The method of claim 58, wherein at least two monoclonal antibodies bind to HER2.

60. The method of claim 59, wherein the IV bag contains a mixture of Trastuzumab and Pertuzumab.

61. The method of claim 34, wherein at least one monoclonal antibody binds to CMV.

62. The method of claim 61, wherein at least two monoclonal antibodies bind to CMV.

63. The method of claim 62, wherein at least one monoclonal antibody binds to HCMV Complex I.

64. The method of claim 63, wherein at least one monoclonal antibody binds to HCMV gH.

65. The method of claim 64, wherein the IV bag contains a mixture of an antibody specifically binding to HCMV gH and an antibody specifically binding to HCMV Complex I.
